(19) United States
(12) Patent Application Publication
(10) Pub. No.: US 2023/0277603 A1
(43) Pub. Date: Sep. 7, 2023

(54) VEILLONELLA PARVULA STRAIN AS AN ORAL THERAPY FOR NEUROINFLAMMATORY DISEASES

(71) Applicant: Evelo Biosciences, Inc., Cambridge, MA (US)

(72) Inventors: Mark Bodmer, Boston, MA (US); Andrea Itano, Arlington, MA (US); Holly Ponichtera, Watertown, MA (US)

(21) Appl. No.: 18/005,148

(22) PCT Filed: Jul. 20, 2021
(86) PCT No.: PCT/US2021/042333

§ 371 Date: Jan. 11, 2023

Related U.S. Application Data
(60) Provisional application No. 63/189,985, filed on May 18, 2021.
(60) Provisional application No. 63/054,533, filed on Jul. 21, 2020.

(51) Int. Cl. A61K 35/74; A61P 25/00; A61K 9/19; A61K 9/00; A61K 31/573; A61K 31/137

(52) U.S. Cl. CPC A61K 35/74; A61K 9/0019; A61K 9/0053; A61K 9/19; A61K 31/137; A61K 31/573; A61P 25/00

(57) ABSTRACT

Provided herein are methods and pharmaceutical compositions related to the bacteria and microbial extracellular vesicles (mEVs) of Veillonella parvula Strain A that are useful as therapeutic agents, such as for treating neuroinflammation.

### CROSS REFERENCE TO RELATED APPLICATION

This application claims the benefit of the following U.S. Provisional Application Serial Nos. 63/054,533, filed Jul. 21, 2020, and 63/189,985, filed May 18, 2021, the entire contents of which are incorporated herein by reference.

### BACKGROUND

Inflammation is an important and appropriate host response to infection or injury. However, dysregulation of this response, with resulting persistent or inappropriate inflammation, underlies a broad range of pathological processes. Collectively, inflammatory disorders including neuroinflammatory diseases, autoimmune diseases, allergies, asthma and sepsis are a major cause of illness and death. It is also becoming apparent that low-grade chronic inflammation underlies many diseases, including type 2 diabetes, cancer, cardiovascular disease and neurodegeneration, that previously were not considered to possess a strong inflammatory component. There is strong interest in identifying new anti-inflammatory drugs for treating neuroinflammation.

### SUMMARY

Provided herein are methods and compositions related to the use of certain strain of Veillonella parvula (e.g., Veillonella parvula Strain A) (e.g. a therapeutically effective amount thereof) and/or its derivatives in the treatment and/or prevention of diseases and disorders (e.g., a neuroinflammatory disease (neuroinflammation), a neurodegenerative disease, a neuromuscular disease, and/or a psychiatric disorder). As disclosed herein, Veillonella parvula (e.g., Veillonella parvula Strain A) bacteria and their derivatives, such as microbial extracellular vesicles (mEVs) (e.g., secreted microbial extracellular vesicles (smEVs) or processed microbial extracellular vesicles (pmEVs)), or any combination thereof, have therapeutic effects and are useful for the treatment and/or prevention of a disease or a health disorder (e.g., a neuroinflammatory disease, a neurodegenerative disease, a neuromuscular disease, and/or a psychiatric disorder).

Current disease modifying strategies to treat neuroinflammatory diseases such as multiple sclerosis include immunomodulatory therapies such as S1P receptor inhibitors (Gilenya), Nrf2 activators (Tecfidera), or IV/SubCu-infused biologics (Ocrevus, Tysabri, Copaxane, Avonex, etc). Veillonella parvula Strain A and/or its derivatives can be used alone or in combination with one of these therapies for neuroinflammation (e.g., multiple sclerosis).

In certain aspects, provided herein are pharmaceutical compositions comprising Veillonella parvula (e.g., Veillonella parvula Strain A) bacteria, Veillonella parvula (e.g., Veillonella parvula Strain A) mEVs (such as smEVs and/or pmEVs), or any combination thereof. In some embodiments, the pharmaceutical compositions provided herein comprise a therapeutically effective amount of Veillonella parvula bacteria, Veillonella parvula mEVs (such as smEVs and/or pmEVs), or any combination thereof.

In some embodiments, a pharmaceutical composition provided herein comprises Veillonella parvula (e.g., Veillonella parvula Strain A) bacteria (e.g., live bacteria, killed bacteria, attenuated bacteria). In some embodiments, a pharmaceutical composition comprises Veillonella parvula (e.g., Veillonella parvula Strain A) mEVs (such as smEVs and/or pmEVs). For example, in some embodiments, a pharmaceutical composition comprises Veillonella parvula smEVs. In some embodiments, a pharmaceutical composition comprises Veillonella parvula pmEVs. In some embodiments, a pharmaceutical composition comprises Veillonella parvula smEVs and Veillonella parvula pmEVs. In some embodiments, a pharmaceutical composition comprises Veillonella parvula bacteria and Veillonella parvula mEVs (such as smEVs and/or pmEVs). For example, in some embodiments, a pharmaceutical composition comprises Veillonella parvula bacteria and Veillonella parvula smEVs. In some embodiments, a pharmaceutical composition comprises Veillonella parvula bacteria and Veillonella parvula pmEVs. In some embodiments, a pharmaceutical composition comprises Veillonella parvula bacteria, Veillonella parvula smEVs, and Veillonella parvula pmEVs.

In some embodiments, a pharmaceutical composition provided herein comprising mEVs can contain smEVs, pmEVs or a combination of both.

In some embodiments, the Veillonella parvula strain is a strain comprising at least 80%, at least 85%, at least 90%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, and/or CRISPR sequence) of the Veillonella parvula Strain A (ATCC Deposit Number PTA-125691).

In some embodiments, the Veillonella parvula strain is the Veillonella parvula Strain A (ATCC Deposit Number PTA-125691).

In some embodiments, a pharmaceutical composition comprises at least 1 × 10⁶, 1 × 10⁷, or 1 × 10⁸ colony forming units (CFUs) of Veillonella parvula (e.g., Veillonella parvula Strain A) whole bacteria. In some embodiments, a pharmaceutical composition comprises at least 1 × 10⁶, 1 × 10⁷, 1 × 10⁸, 1 × 10⁹, 1 × 10¹⁰, 1 × 10¹¹, or 1 × 10¹² total cell count (TCC) of Veillonella parvula (e.g., Veillonella parvula Strain A) whole bacteria. TCC can be determined e.g., by Coulter counter.

In some embodiments, the whole bacteria may be live, killed, attenuated, lyophilized, or irradiated (e.g., UV or gamma irradiated). In some embodiments, the whole bacteria is irradiated (e.g., gamma irradiated).

In some embodiments, a pharmaceutical composition comprises secreted mEVs (smEVs) from Veillonella parvula (e.g., Veillonella parvula Strain A). In some embodiments, a pharmaceutical composition comprises processed mEVs (pmEVs) of Veillonella parvula (e.g., Veillonella parvula Strain A).

In some embodiments, a pharmaceutical composition comprises pmEVs and the pmEVs are produced from live bacteria. In some embodiments, the pmEVs are produced from dead bacteria. In some embodiments, the pmEVs are produced from bacteria that have been gamma irradiated, UV irradiated, heat inactivated, acid treated, or oxygen sparged. In some embodiments, the pmEVs are produced from non-replicating bacteria.

In some embodiments, a pharmaceutical composition comprises mEVs (such as smEVs and/or pmEVs) that are lyophilized (e.g., the lyophilized product further comprises a pharmaceutically acceptable excipient). In some embodiments, the mEVs (such as smEVs and/or pmEVs) are gamma irradiated. In some embodiments, the mEVs (such as smEVs and/or pmEVs) are UV irradiated. In some embodiments, the mEVs (such as smEVs and/or pmEVs) are heat inactivated (e.g., at 50° C. for two hours or at 90° C. for two hours). In some embodiments, the mEVs (such as smEVs and/or pmEVs) are acid treated. In some embodiments, the mEVs (such as smEVs and/or pmEVs) are oxygen sparged (e.g., at 0.1 vvm for two hours).

In some embodiments, a pharmaceutical composition comprises a dose of mEVs (such as smEVs and/or pmEVs) of about 2×10⁶ to about 2×10¹⁶ particles (e.g., wherein particle count is determined by NTA (nanoparticle tracking analysis)). In some embodiments, the dose of mEVs (such as smEVs and/or pmEVs) is about 1×10⁷- about 1×10¹⁵ particles, e.g., as measured by NTA. In some embodiments, a pharmaceutical composition comprises a dose of mEVs (such as smEVs and/or pmEVs) of about 5 mg to about 900 mg total protein (e.g., wherein total protein is determined by Bradford assay or BCA).

In certain aspect, a pharmaceutical composition provided herein comprises Veillonella parvula (e.g., Veillonella parvula Strain A) microbial extracellular vesicles (mEVs) (such as smEVs and/or pmEVs) and Veillonella parvula (e.g., Veillonella parvula Strain A) bacteria.

In some embodiments, the pharmaceutical composition comprises smEVs and the smEVs are produced from live bacteria. In some embodiments, the pharmaceutical composition comprises smEVs and the smEVs are produced from a high yield strain of Veillonella parvula (e.g., Veillonella parvula Strain A) bacteria.

In some embodiments, a pharmaceutical composition comprises at least, about, or no more than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the total particles in the pharmaceutical composition are Veillonella parvula (e.g., Veillonella parvula Strain A) mEVs (such as smEVs and/or pmEVs).

In some embodiments, a pharmaceutical composition comprises at least, about, or no more than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the total particles in the pharmaceutical composition are Veillonella parvula (e.g., Veillonella parvula Strain A) bacteria particles.

In some embodiments, a pharmaceutical composition comprises at least, about, or no more than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the total proteins in the pharmaceutical composition are Veillonella parvula (e.g., Veillonella parvula Strain A) mEVs (such as smEVs and/or pmEVs).

In some embodiments, a pharmaceutical composition comprises at least, about, or no more than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the total proteins in the pharmaceutical composition are Veillonella parvula (e.g., Veillonella parvula Strain A) bacteria proteins.

In some embodiments, a pharmaceutical composition comprises at least, about, or no more than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the total lipids in the pharmaceutical composition are Veillonella parvula (e.g., Veillonella parvula Strain A) mEVs (such as smEVs and/or pmEVs).

In some embodiments, a pharmaceutical composition comprises at least, about, or no more than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the total lipids in the pharmaceutical composition are Veillonella parvula (e.g., Veillonella parvula Strain A) bacteria lipids.

In certain aspects, a pharmaceutical composition provided herein comprising Veillonella parvula (e.g., Veillonella parvula Strain A) bacteria, mEVs (such as smEVs and/or pmEVs), or any combination thereof, can be used for the treatment or prevention of a disease in a subject. In some embodiments, the disease is a neuroinflammatory disease, a neurodegenerative disease, a neuromuscular disease, and/or a psychiatric disorder.

In some embodiments, the pharmaceutical composition described herein is administered once a day. In some embodiments, the pharmaceutical composition described herein is administered twice a day. In some embodiments, the pharmaceutical composition described herein is formulated for a daily dose. In some embodiments, the pharmaceutical composition described herein is formulated for twice a day dose, wherein each dose is half of the daily dose.

In some embodiments, a pharmaceutical composition provided herein induces an immune response. In some embodiments, a pharmaceutical composition reduces inflammation, optionally neuroinflammation. In some embodiments, a pharmaceutical composition activates innate antigen presenting cells.

In certain aspects, a pharmaceutical composition provided herein comprising Veillonella parvula (e.g., Veillonella parvula Strain A) bacteria, mEVs (such as smEVs and/or pmEVs), or any combination thereof, is formulated for oral, rectal, sublingual, intradermal, intravenous, intraperitoneal, or subcutaneous administration.

In some embodiments, a pharmaceutical composition provided herein comprising Veillonella parvula (e.g., Veillonella parvula Strain A) bacteria, mEVs (such as smEVs and/or pmEVs), or any combination thereof, can be prepared as powder (e.g., for resuspension) or as a solid dose form, such as a tablet, a minitablet, a capsule, a pill, or a powder; or a combination of these forms (e.g., minitablets comprised in a capsule). In some embodiments, the pharmaceutical composition further comprises a pharmaceutically acceptable excipient.

In some embodiments, a pharmaceutical composition provided herein can comprise lyophilized Veillonella parvula (e.g., Veillonella parvula Strain A) bacteria, mEVs (such as smEVs and/or pmEVs), or any combination thereof. The lyophilized Veillonella parvula bacteria, mEVs (such as smEVs and/or pmEVs), or any combination thereof, can be formulated into a solid dose form (optionally comprising an enteric coating), such as a tablet, a minitablet, a capsule, a pill, or a powder; or can be resuspended in a solution (optionally further comprising a pharmaceutical excipient (e.g., sucrose or glucose)).

In some embodiments, a pharmaceutical composition provided herein can comprise gamma irradiated Veillonella parvula (e.g., Veillonella parvula Strain A) bacteria, mEVs (such as smEVs and/or pmEVs), or any combination thereof. The gamma irradiated Veillonella parvula bacteria, mEVs (such as smEVs and/or pmEVs), or any combination thereof, can be formulated into a solid dose form (optionally comprising an enteric coating), such as a tablet, a minitablet, a capsule, a pill, or a powder; or can be resuspended in a solution (optionally further comprising a pharmaceutical excipient (e.g., sucrose or glucose)).

In some embodiments, a pharmaceutical composition provided herein comprising Veillonella parvula (e.g., Veillonella parvula Strain A) bacteria, mEVs (such as smEVs and/or pmEVs), or any combination thereof, can be orally administered. In some embodiments, a pharmaceutical composition provided herein comprising Veillonella parvula bacteria, mEVs (such as smEVs and/or pmEVs), or any combination thereof, can be administered intravenously. In some embodiments, a pharmaceutical composition provided herein comprising Veillonella parvula bacteria, mEVs (such as smEVs and/or pmEVs), or any combination thereof, can be administered rectally, sublingually, intradermally, intravenously, intraperitoenally, or subcutaneously. In some embodiments, a pharmaceutical composition provided herein comprising Veillonella parvula bacteria, mEVs (such as smEVs and/or pmEVs), or any combination thereof, can be administered topically.

In certain aspects, provided herein are methods of making and/or identifying Veillonella parvula (e.g., Veillonella parvula Strain A) bacteria, mEVs (such as smEVs and/or pmEVs), or any combination thereof, that are useful for treatment and/or prevention of a disease.

In certain aspects, provided herein is use of a pharmaceutical composition described herein for the preparation of a medicament for treatment (or prevention) of a condition described herein, e.g., a neuroinflammatory disease, a neurodegenerative disease, a neuromuscular disease, and/or a psychiatric disorder, e.g., as described herein.

In certain aspects, provided herein is a pharmaceutical composition described herein for use in treating (or preventing) of a condition described herein, e.g., a neuroinflammatory disease, a neurodegenerative disease, a neuromuscular disease, and/or a psychiatric disorder, e.g., as described herein.

In certain aspects, provided herein is a method of using the pharmaceutical compositions described herein in treating a subject (e.g., human) in need thereof.

In some embodiments, a method treats or prevents a disease in a subject, the method comprising administering to the subject at least one pharmaceutical composition described herein. Non-limiting examples of the disease include a neuroinflammatory disease, a neurodegenerative disease, a neuromuscular disease, and/or a psychiatric disorder.

In some embodiments, the subject is a mammal. In some embodiments, the subject is a human.

In some embodiments, the pharmaceutical composition described herein is administered once a day. In some embodiments, the pharmaceutical composition described herein is administered twice a day. In some embodiments, the pharmaceutical composition described herein is formulated for a daily dose. In some embodiments, the pharmaceutical composition described herein is formulated for twice a day dose, wherein each dose is half of the daily dose.

In some embodiments, a pharmaceutical composition and/or a method described herein is used to treat a disease selected from encephalitis, encephalomyelitis, meningitis, Guillain-Barre syndrome, neuromyotonia, narcolepsy, multiple sclerosis, myelitis, schizophrenia, acute disseminated encephalomyelitis (ADEM), acute optic neuritis (AON), transverse myelitis, neuromyelitis optica (NMO), Alzheimer’s disease, Parkinson’s disease, amyotrophic lateral sclerosis, frontotemporal lobar dementia, optic neuritis, neuromyelitis optica spectrum disorder (NMOSD), autoimmune encephalitis, anti-NMDA receptor encephalitis, Rasmussen’s encephalitis, acute necrotizing encephalopathy of childhood (ANEC), opsoclonus-myoclonus ataxia syndrome, traumatic brain injury, Huntington’s disease, depression, anxiety, migraine, myasthenia gravis, acute ischemic stroke, epilepsy, synucleinopathies, frontotemporal dementia, progressive nonfluent aphasia, semantic dementia, Nodding syndrome, cerebral ischemia, neuropathic pain, autism spectrum disorder, fibromyalgia syndrome, progressive supranuclear palsy, corticobasal degeneration, systemic lupus erythematosus, prion disease, motor neuron diseases (MND), spinocerebellar ataxia, spinal muscular atrophy, dystonia, idiopathicintracranial hypertension, nervous system disease, central nervous system disease, peripheral nervous system disease, movement disorders, encephalopathy, peripheral neuropathy, and post-operative cognitive dysfunction.

In some embodiments, a pharmaceutical composition and/or a method described herein is used to treat a disease selected from, encephalitis, encephalomyelitis, meningitis, multiple sclerosis, schizophrenia, acute disseminated encephalomyelitis (ADEM), accute optic neuritis (AON), transverse myelitis, neuromyelitis optica (NMO), Alzheimer’s disease, Parkinson’s disease, amyotrophic lateral sclerosis, frontotemporal lobar dementia, traumatic brain injury, Huntington’s disease, depression, anxiety, migraine, acute ischemic stroke, epilepsy, synucleinopathies, semantic dementia, cerebral ischemia, neuropathic pain, autism spectrum disorder, peripheral neuropathy, motor neuron diseases (MND), spinocerebellar ataxia, spinal muscular atrophy, and fibromyalgia syndrome. In some embodiments, the disease is multiple sclerosis.

In some embodiments, the method or use reduces disease score in EAE model of disease, e.g., as described herein. In some embodiments, the method or use reduces disease score in the acute phase in EAE model, e.g., as described herein. In some embodiments, the method or use reduces disease score in the relapsing-remitting phase in EAE model, e.g., as described herein.

In some embodiments, the method or use reduces AUC during relapsing-remitting phase in EAE model of disease, e.g., as described herein.

In some embodiments, the method or use reduces demyelination of the spinal cord in EAE model of disease, e.g., as described herein.

In some embodiments, the method or use does not reduce inflammation of the spinal cord in EAE model of disease, e.g., as described herein.

In some embodiments, the method or use reduces inflammation in the spinal cord (e.g., in EAE model of disease), e.g., as described herein. In some embodiments, the method or use reduces inflammation in the cervical spinal cord, e.g., as described herein. In some embodiments, the method or use reduces inflammation in the thoracic spinal cord, e.g., as described herein. In some embodiments, the method or use reduces inflammation in the lumbar spinal cord, e.g., as described herein.

In some embodiments, the method or use reduces AUC of EAE with therapeutic dosing (e.g., after the onset of disease), e.g., as described herein. In some embodiments, the method or use reduces AUC during relapsing-remitting phase of EAE with therapeutic dosing (e.g., after the onset of disease), e.g., as described herein.

In some embodiments, a method or use of a pharmaceutical composition provided herein further comprises administering to the subject one or more additional therapeutic agents. In some embodiments, a pharmaceutical composition further comprises one or more additional therapeutic agents. In some embodiments, the one or more additional therapeutic agents is an immunotherapy and/or an immune modulating protein (e.g., an immune checkpoint inhibitor, an antibody, a vaccine, a primed antigen presenting cell, a T cell, an immune activating protein, a cytokine, and/or an adjuvant). In some embodiments, the one or more therapeutic agents is another therapeutic bacteria, mEVs (such as smEVs and/or pmEVs), or any combination thereof, from one or more other bacterial strains (e.g., therapeutic bacteria). In some embodiments, the one or more therapeutic agents is an immune suppressant and/or an anti-inflammatory agent. In some embodiments, the one or more therapeutic agents is a metabolic disease therapeutic agent.

In some embodiments, the one or more additional therapeutic agents is selected from the group consisting of an immunosuppressive agent, a DMARD, a pain-control drug, a steroid, a non-steroidal anti-inflammatory drug (NSAID), a cytokine antagonist, cyclosporin, retinoids, corticosteroids, propionic acid derivative, acetic acid derivative, enolic acid derivatives, fenamic acid derivatives, Cox-2 inhibitors, lumiracoxib, ibuprofen, choline magnesium salicylate, fenoprofen, salsalate, difunisal, tolmetin, ketoprofen, flurbiprofen, oxaprozin, indomethacin, sulindac, etodolac, ketorolac, nabumetone, naproxen, valdecoxib, etoricoxib, MK0966; rofecoxib, acetaminophen, Celecoxib, Diclofenac, tramadol, piroxicam, meloxicam, tenoxicam, droxicam, lornoxicam, isoxicam, mefanamic acid, meclofenamic acid, flufenamic acid, tolfenamic, valdecoxib, parecoxib, etodolac, indomethacin, aspirin, ibuprophen, firocoxib, methotrexate (MTX), antimalarial drugs, hydroxychloroquine, chloroquine, sulfasalazine, Leflunomide, azathioprine, cyclosporin, gold salts, minocycline, cyclophosphamide, D-penicillamine, minocycline, auranofin, tacrolimus, myocrisin, chlorambucil, TNF alpha antagonists, TNF alpha antagonists, TNF alpha receptor antagonists, ADALIMUMAB (Humira®), ETANERCEPT (Enbrel®), INFLIXIMAB (Remicade®; TA-650), CERTOLIZUMAB PEGOL (Cimzia®; CDP870), GOLIMUMAB (Simpom®; CNTO 148), ANAKINRA (Kineret®), RITUXIMAB (Rituxan®; MabThera®), ABATACEPT (Orencia®), TOCILIZUMAB (RoActemra /Actemra®), integrin antagonists, TYSABRI® (natalizumab), IL-1 antagonists, ACZ885 (Ilaris), Anakinra (Kineret®), CD4 antagonists, IL-23 antagonists, IL-20 antagonists, IL-6 antagonists, BLyS antagonists, Atacicept, Benlysta®/ LymphoStat-B® (belimumab), p38 Inhibitors, CD20 antagonists, Ocrelizumab, Ofatumumab (Arzerrat), interferon gamma antagonists, Fontolizumab, prednisolone, Prednisone, dexamethasone, Cortisol, cortisone, hydrocortisone, methylprednisolone, betamethasone, triamcinolone, beclometasome, fludrocortisone, deoxycorticosterone, aldosterone, Doxycycline, vancomycin, pioglitazone, SBI-087, SCIO-469, Cura-100, Oncoxin + Viusid, TwHF, Methoxsalen, Vitamin D - ergocalciferol, Milnacipran, Paclitaxel, rosig tazone, Tacrolimus, Prograf®, RADOO1, rapamune, rapamycin, fostamatinib, Fentanyl, XOMA 052, Fostamatinib disodium, rosightazone, Curcumin, Longvida™, Rosuvastatin, Maraviroc, ramipnl, Milnacipran, Cobiprostone, somatropin, tgAAC94 gene therapy vector, MK0359, GW856553, esomeprazole, everolimus, trastuzumab, JAK1 and JAK2 inhibitors, pan JAK inhibitors, e.g., tetracyclic pyridone 6 (P6), 325, PF-956980, denosumab, IL-6 antagonists, CD20 antagonistis, CTLA4 antagonists, IL-8 antagonists, IL-21 antagonists, IL-22 antagonist, integrin antagonists, Tysarbri® (natalizumab), VGEF antagnosits, CXCL antagonists, MMP antagonists, defensin antagonists, IL-1 antagonists, IL-1 beta antagonsits, IL-23 antagonists, receptor decoys, antagonistic antibodies, corticosteroids, mesalazine, mesalamine, sulfasalazine, sulfasalazine derivatives, immunosuppressive drugs, cyclosporin A, mercaptopurine, azathiopurine, prednisone, methotrexate, antihistamines, glucocorticoids, epinephrine, theophylline, cromolyn sodium, anti-leukotrienes, anti-cholinergic drugs for rhinitis, TLR antagonists, inflammasome inhibitors, anti-cholinergic decongestants, mast-cell stabilizers, monoclonal anti-IgE antibodies, vaccines, cytokine inhibitors, anti-IL-6 antibodies, TNF inhibitors, palmitoylethanolamide, an inhibitor of N-Acylethanolamine Acid Amidase (NAAA), interferon-β, glatiramer acetate, mitoxantrone, and glucocorticoids.

In some embodiments, the one or more additional therapeutic agents is selected from the group consisting of an immunosuppressive agent, a non-steroidal anti-inflammatory drug (NSAID), palmitoylethanolamide, an inhibitor of N-Acylethanolamine Acid Amidase (NAAA), interferon-β, glatiramer acetate, mitoxantrone, and glucocorticoids.

In some embodiments, the one or more additional therapeutic agents is selected from the group consisting of an S1P receptor inhibitor (such as Gilenya), a Nrf2 activator (such as Tecfidera), or a biologic (e.g., IV/SubCu-infused biologic) (such as Ocrevus, Tysabri, Copaxane, or Avonex).

In some embodiments, the one or more additional therapeutic agents is selected from the group consisting of secukinumab, ustikinumab, and bimekizumab.

In some embodiments, the one or more additional therapeutic agents is an antibiotic. In some embodiments, the antibiotic is selected from the group consisting of aminoglycosides, ansamycins, carbacephems, carbapenems, cephalosporins, glycopeptides, lincosamides, lipopeptides, macrolides, monobactams, nitrofurans, oxazolidonones, penicillins, polypeptide antibiotics, quinolones, fluoroquinolone, sulfonamides, tetracyclines, anti-mycobacterial compounds and combinations thereof.

In certain aspects, further provided herein is a method of preparing a pharmaceutical composition described herein in a suspension, the method comprising: combining Veillonella parvula (e.g., Veillonella parvula Strain A) mEVs, bacteria, or any combination thereof, with a pharmaceutically acceptable buffer (e.g., PBS); thereby preparing the pharmaceutical composition. In some embodiments, the suspension further comprises sucrose or glucose.

In certain aspects, also provided herein is a method of preparing a pharmaceutical composition described herein in a solid dose form, the method comprising: (a) combining Veillonella parvula (e.g., Veillonella parvula Strain A) mEVs, bacteria, or any combination thereof, with a pharmaceutically acceptable excipient, and (b) compressing the Veillonella parvula (e.g., Veillonella parvula Strain A) mEVs, bacteria, or any combination thereof; and a pharmaceutically acceptable excipient, thereby preparing the pharmaceutical composition. In some embodiments, the method further comprises enterically coating the solid dose form.

A pharmaceutical composition provided herein can deliver a therapeutically effective amount of Veillonella parvula (e.g., Veillonella parvula Strain A) bacteria, mEVs (such as smEVs and/or pmEVs), or any combination thereof, to a subject (e.g., a human) in need thereof. Similarly, a pharmaceutical composition provided herein can deliver a non-natural amount of the therapeutically effective amount of Veillonella parvula (e.g., Veillonella parvula Strain A) bacteria, mEVs (such as smEVs and/or pmEVs), or any combination thereof, to a subject (e.g., a human) in need thereof. Such pharmaceutical composition can bring benefits to a subject (e.g., a human), such as treating and/or preventing a disease or a healthy disorder.

### BRIEF DESCRIPTION OF THE FIGURES

FIG. 1 is a graph showing the disease score summary for a 42 day SJL EAE study with prophylactic treatment.

FIG. 2A and FIG. 2B are graphs showing that Strain A reduced disease score in the acute phase and relapsing-remitting phase. FIG. 2A shows the disease score. FIG. 2B shows the total area under the curve.

FIGS. 3A-3D are graphs showing that Strain A reduces inflammation of the spinal cord. FIG. 3A shows inflammation in the cervical spinal cord based on histopathological analysis of inflammatory foci in H&E stained tissue sections. FIG. 3B displays inflammation in the thoracic spinal cord. FIG. 3C displays inflammation in the lumbar spinal cord. FIG. 3D displays average inflammation in the cervical, thoracic, and lumbar spinal cord regions. Dots represent individual mice. Statistical analysis was performed using a Student’s t-test.

FIG. 4 is a graph showing the disease score summary for a 42 day SJL EAE study with therapeutic treatment.

FIGS. 5A-5C are graphs showing that therapeutic treatment with Strain A reduces AUC during relapsing-remitting phase of EAE. FIG. 5A displays the AUC for the total study. FIG. 5B displays the AUC for the acute phase (days 0-20). FIG. 5C displays the AUC for the relapse phase (days 20-42).

FIG. 6 is a graph showing the disease score summary for a 42 day SJL EAE study with therapeutic treatment with Strain A.

FIGS. 7A-7C are graphs showing that therapeutic treatment with Strain A reduces AUC during relapsing-remitting phase of EAE. FIG. 7A displays the AUC for the total study (days 0-42). FIG. 7B displays the AUC for the acute phase (days 0-17). FIG. 7C displays the AUC for the relapse phase (days 18-42). Bars show mean + SEM. Statistical analysis performed using a two-tailed unpaired t-test.

FIGS. 8A-8D are graphs showing that therapeutic treatment with Strain A does not significantly reduce inflammation of the spinal cord based on histopathological analysis of inflammatory foci in H&E stained tissue sections. FIGS. 8A-D display that inflammation was reduced, although not to a level reaching statistical significance in mice treated with Strain A compared to mice treated with vehicle. Dots represent individual mice. Bars show mean ± SEM. Statistical analysis was performed using a one-way ANOVA with Dunnett’s multiple comparisons. FIG. 8A displays average inflammation in the cervical, thoracic, and lumbar spinal cord regions in mice treated with Strain A, fingolimod, or vehicle. FIG. 8C displays reduced inflammation, although not to a level reaching statistical significance, in the thoracic spinal cord region in mice treated with Strain A compared to vehicle. FIG. 8D displays that inflammation was reduced, although not to a level reaching statistical significance, in the lumbar spinal cord region in mice treated with Strain A compared to vehicle.

FIGS. 9A-9D are graphs showing that therapeutic treatment with Strain A reduces demyelination of the spinal cord. In these figures, dots represent individual mice. Bars show mean ± SEM. Statistical analysis was performed using a one-way ANOVA with Dunnett’s multiple comparisons. FIG. 9A shows a reduced average demyelination score in the cervical, thoracic, and lumbar spinal cord regions from mice treated with Strain A relative to vehicle control. FIG. 9B shows a non-significant change in the demyelination score in the cervical region in mice treated with Strain A relative to vehicle control. FIG. 9C shows significant reduction in demyelination scores in the thoracic region of the spine of the mice treated with Strain A relative to vehicle control. FIG. 9D shows significant reduction in demyelination scores in the lumbar region of the spine of the mice treated with Strain A relative to vehicle control.

### DETAILED DESCRIPTION

In certain aspects, provided herein are methods and compositions related to the use of Veillonella parvula (e.g., Veillonella parvula Strain A) bacteria and its derivatives (e.g., mEVs, such as smEVs and/or pmEVs) in the treatment and/or prevention of a disease or a health disorder (e.g., a neuroinflammatory disease, a neurodegenerative disease, a neuromuscular disease, and/or a psychiatric disorder).

Preclinical data support the use of oral gamma irradiated Veillonella parvula Strain A for neuroinflammatory diseases such as multiple sclerosis.

Current disease modifying strategies to treat neuro-inflammatory diseases such as multiple sclerosis include immunomodulatory therapies such as S1P receptor inhibitors (Gilenya), Nrf2 activators (Tecfidera), or IV/SubCu-infused biologics (Ocrevus, Tysabri, Copaxane, Avonex, etc). Veillonella parvula Strain A (e.g., gamma irradiated Strain A) can be used alone or in combination with one of these therapies for neuro-inflammation (e.g., a neuroinflammatory disease, a neurodegenerative disease, a neuromuscular disease, and/or a psychiatric disorder) (e.g., multiple sclerosis).

### Definitions

“Adjuvant” or “Adjuvant therapy” broadly refers to an agent that affects an immunological or physiological response in a patient or subject (e.g., human). For example, an adjuvant might increase the presence of an antigen over time or to an area of interest, help absorb an antigen presenting cell antigen, activate macrophages and lymphocytes and support the production of cytokines. By changing an immune response, an adjuvant might permit a smaller dose of an immune interacting agent to increase the effectiveness or safety of a particular dose of the immune interacting agent. For example, an adjuvant might prevent T cell exhaustion and thus increase the effectiveness or safety of a particular immune interacting agent.

“Administration” broadly refers to a route of administration of a composition (e.g., a pharmaceutical composition) to a subject. Examples of routes of administration include oral administration, rectal administration, topical administration, inhalation (nasal) or injection. Administration by injection includes intravenous (IV), intramuscular (IM), and subcutaneous (SC) administration. A pharmaceutical composition described herein can be administered in any form by any effective route, including but not limited to oral, parenteral, enteral, intravenous, intraperitoneal, topical, transdermal (e.g., using any standard patch), intradermal, ophthalmic, (intra)nasally, local, non-oral, such as aerosol, inhalation, subcutaneous, intramuscular, buccal, sublingual, (trans)rectal, vaginal, intraarterial, and intrathecal, transmucosal (e.g., sublingual, lingual, (trans)buccal, (trans)urethral, vaginal (e.g., trans- and perivaginally), implanted, intravesical, intrapulmonary, intraduodenal, intragastrical, and intrabronchial. In preferred embodiments, a pharmaceutical composition described herein is administered orally, rectally, topically, intravesically, by injection into or adjacent to a draining lymph node, intravenously, by inhalation or aerosol, or subcutaneously. In another preferred embodiment, a pharmaceutical composition described herein is administered orally.

As used herein, the term “antibody” may refer to both an intact antibody and an antigen binding fragment thereof. Intact antibodies are glycoproteins that include at least two heavy (H) chains and two light (L) chains inter-connected by disulfide bonds. Each heavy chain includes a heavy chain variable region (abbreviated herein as V_{H}) and a heavy chain constant region. Each light chain includes a light chain variable region (abbreviated herein as V_{L}) and a light chain constant region. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The term “antibody” includes, for example, monoclonal antibodies, polyclonal antibodies, chimeric antibodies, humanized antibodies, human antibodies, multispecific antibodies (e.g., bispecific antibodies), single-chain antibodies and antigen-binding antibody fragments.

The terms “antigen binding fragment” and “antigen-binding portion” of an antibody, as used herein, refer to one or more fragments of an antibody that retain the ability to bind to an antigen. Examples of binding fragments encompassed within the term “antigen-binding fragment” of an antibody include Fab, Fab′, F(ab′)₂, Fv, scFv, disulfide linked Fv, Fd, diabodies, single-chain antibodies, NANOBODIES®, isolated CDRH3, and other antibody fragments that retain at least a portion of the variable region of an intact antibody. These antibody fragments can be obtained using conventional recombinant and/or enzymatic techniques and can be screened for antigen binding in the same manner as intact antibodies.

A “carbohydrate” refers to a sugar or polymer of sugars. The terms “saccharide,” “polysaccharide,” “carbohydrate,” and “oligosaccharide” may be used interchangeably. Most carbohydrates are aldehydes or ketones with many hydroxyl groups, usually one on each carbon atom of the molecule. Carbohydrates generally have the molecular formula CₙH₂ₙOₙ. A carbohydrate may be a monosaccharide, a disaccharide, trisaccharide, oligosaccharide, or polysaccharide. The most basic carbohydrate is a monosaccharide, such as glucose, galactose, mannose, ribose, arabinose, xylose, and fructose. Disaccharides are two joined monosaccharides. Exemplary disaccharides include sucrose, maltose, cellobiose, and lactose. Typically, an oligosaccharide includes between three and six monosaccharide units (e.g., raffinose, stachyose), and polysaccharides include six or more monosaccharide units. Exemplary polysaccharides include starch, glycogen, and cellulose. Carbohydrates may contain modified saccharide units such as 2′-deoxyribose wherein a hydroxyl group is removed, 2′-fluororibose wherein a hydroxyl group is replaced with a fluorine, or N-acetylglucosamine, a nitrogen-containing form of glucose (e.g., 2′-fluororibose, deoxyribose, and hexose). Carbohydrates may exist in many different forms, for example, conformers, cyclic forms, acyclic forms, stereoisomers, tautomers, anomers, and isomers.

“Cellular augmentation” broadly refers to the influx of cells or expansion of cells in an environment that are not substantially present in the environment prior to administration of a composition and not present in the composition itself. Cells that augment the environment include immune cells, stromal cells, bacterial and fungal cells.

“Clade” refers to the OTUs or members of a phylogenetic tree that are downstream of a statistically valid node in a phylogenetic tree. The clade comprises a set of terminal leaves in the phylogenetic tree that is a distinct monophyletic evolutionary unit and that share some extent of sequence similarity.

The term “decrease” or “deplete” means a change, such that the difference is, depending on circumstances, at least 10%, 20%, 30%, 40%, 50%, 60%, 70%, 80%, 90%, 1/100, 1/1000, 1/10,000, 1/100,000, 1/1,000,000 or undetectable after treatment when compared to a pre-treatment state. Properties that may be decreased include the number of immune cells, bacterial cells, stromal cells, myeloid derived suppressor cells, fibroblasts, metabolites; the level of a cytokine; or another physical parameter (such as ear thickness (e.g., in a DTH animal model).

The term “ecological consortium” is a group of bacteria which trades metabolites and positively co-regulates one another, in contrast to two bacteria which induce host synergy through activating complementary host pathways for improved efficacy.

As used herein, “engineered bacteria” are any bacteria that have been genetically altered from their natural state by human activities, and the progeny of any such bacteria. Engineered bacteria include, for example, the products of targeted genetic modification, the products of random mutagenesis screens and the products of directed evolution.

The term “epitope” means a protein determinant capable of specific binding to an antibody or T cell receptor. Epitopes usually consist of chemically active surface groupings of molecules such as amino acids or sugar side chains. Certain epitopes can be defined by a particular sequence of amino acids to which an antibody is capable of binding.

The term “gene” is used broadly to refer to any nucleic acid associated with a biological function. The term “gene” applies to a specific genomic sequence, as well as to a cDNA or an mRNA encoded by that genomic sequence.

“Identity” as between nucleic acid sequences of two nucleic acid molecules can be determined as a percentage of identity using known computer algorithms such as the “FASTA” program, using for example, the default parameters as in Pearson et al. (1988) Proc. Natl. Acad. Sci. USA 85:2444 (other programs include the GCG program package (Devereux, J., et al., Nucleic Acids Research 12(I):387 (1984)), BLASTP, BLASTN, FASTA Atschul, S. F., et al., J Molec Biol 215:403 (1990); Guide to Huge Computers, Mrtin J. Bishop, ed., Academic Press, San Diego, 1994, and Carillo et al. (1988) SIAM J Applied Math 48:1073). For example, the BLAST function of the National Center for Biotechnology Information database can be used to determine identity. Other commercially or publicly available programs include, DNAStar “MegAlign” program (Madison, Wis.) and the University of Wisconsin Genetics Computer Group (UWG) “Gap” program (Madison Wis.)).

As used herein, the term “immune disorder” refers to any disease, disorder or disease symptom caused by an activity of the immune system, including autoimmune diseases, inflammatory diseases and allergies. Immune disorders include, but are not limited to, autoimmune diseases (e.g., psoriasis, atopic dermatitis, lupus, scleroderma, hemolytic anemia, vasculitis, type one diabetes, Grave’s disease, rheumatoid arthritis, multiple sclerosis, Goodpasture’s syndrome, pernicious anemia and/or myopathy), inflammatory diseases (e.g., acne vulgaris, asthma, celiac disease, chronic prostatitis, glomerulonephritis, inflammatory bowel disease, pelvic inflammatory disease, reperfusion injury, rheumatoid arthritis, sarcoidosis, transplant rejection, vasculitis and/or interstitial cystitis), and/or an allergies (e.g., food allergies, drug allergies and/or environmental allergies).

“Immunotherapy” is treatment that uses a subject’s immune system to treat disease (e.g., immune disease, inflammatory disease, autoimmune disease) and includes, for example, checkpoint inhibitors, vaccines, cytokines, cell therapy, and dendritic cell therapy.

The term “increase” means a change, such that the difference is, depending on circumstances, at least 10%, 20%, 30%, 40%, 50%, 60%, 70%, 80%, 90%, 2-fold, 4-fold, 10-fold, 100-fold, 10^3 fold, 10^4 fold, 10^5 fold, 10^6 fold, and/or 10^7 fold greater after treatment when compared to a pre-treatment state. Properties that may be increased include the number of immune cells, bacterial cells, stromal cells, myeloid derived suppressor cells, fibroblasts, metabolites; the level of a cytokine; or another physical parameter (such as ear thickness (e.g., in a DTH animal model)).

“Innate immune agonists” or “immuno-adjuvants” are small molecules, proteins, or other agents that specifically target innate immune receptors including Toll-Like Receptors (TLR), NOD receptors, RLRs, C-type lectin receptors, STING-cGAS Pathway components, inflammasome complexes. For example, LPS is a TLR-4 agonist that is bacterially derived or synthesized and aluminum can be used as an immune stimulating adjuvant. immuno-adjuvants are a specific class of broader adjuvant or adjuvant therapy. Examples of STING agonists include, but are not limited to, 2′3′- cGAMP, 3′3′-cGAMP, c-di-AMP, c-di-GMP, 2′2′-cGAMP, and 2′3′-cGAM(PS)2 (Rp/Sp) (Rp, Sp-isomers of the bis-phosphorothioate analog of 2′3′-cGAMP). Examples of TLR agonists include, but are not limited to, TLR1, TLR2, TLR3, TLR4, TLR5, TLR6, TLR7, TLR8, TLR9, TLR10 and TLRI 1. Examples of NOD agonists include, but are not limited to, N-acetylmuramyl-L-alanyl-D-isoglutamine (muramyldipeptide (MDP)), gamma-D-glutamyl-mesodiaminopimelic acid (iE-DAP), and desmuramylpeptides (DMP).

The “internal transcribed spacer” or “ITS” is a piece of non-functional RNA located between structural ribosomal RNAs (rRNA) on a common precursor transcript often used for identification of eukaryotic species in particular fungi. The rRNA of fungi that forms the core of the ribosome is transcribed as a signal gene and consists of the 8S, 5.8S and 28S regions with ITS4 and 5 between the 8S and 5.8S and 5.8S and 28S regions, respectively. These two intercistronic segments between the 18S and 5.8S and 5.8S and 28S regions are removed by splicing and contain significant variation between species for barcoding purposes as previously described (Schoch et al Nuclear ribosomal internal transcribed spacer (ITS) region as a universal DNA barcode marker for Fungi. PNAS 109:6241-6246. 2012). 18S rDNA is traditionally used for phylogenetic reconstruction however the ITS can serve this function as it is generally highly conserved but contains hypervariable regions that harbor sufficient nucleotide diversity to differentiate genera and species of most fungus.

The term “isolated” or “enriched” encompasses a microbe, an mEV (such as an smEV and/or pmEV) or other entity or substance that has been (1) separated from at least some of the components with which it was associated when initially produced (whether in nature or in an experimental setting), and/or (2) produced, prepared, purified, and/or manufactured by the hand of man. Isolated microbes or mEVs may be separated from at least about 10%, about 20%, about 30%, about 40%, about 50%, about 60%, about 70%, about 80%, about 90%, or more of the other components with which they were initially associated. In some embodiments, isolated microbes or mEVs are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. The terms “purify,” “purifying” and “purified” refer to a microbe or mEV or other material that has been separated from at least some of the components with which it was associated either when initially produced or generated (e.g., whether in nature or in an experimental setting), or during any time after its initial production. A microbe or a microbial population or mEV may be considered purified if it is isolated at or after production, such as from a material or environment containing the microbe or microbial population or mEV, and a purified microbe or microbial or mEV population may contain other materials up to about 10%, about 20%, about 30%, about 40%, about 50%, about 60%, about 70%, about 80%, about 90%, or above about 90% and still be considered “isolated.” In some embodiments, purified microbes or mEVs or microbial population are more than about 80%, about 85%, about 90%, about 91%, about 92%, about 93%, about 94%, about 95%, about 96%, about 97%, about 98%, about 99%, or more than about 99% pure. In the instance of microbial compositions provided herein, the one or more microbial types present in the composition can be independently purified from one or more other microbes produced and/or present in the material or environment containing the microbial type. Microbial compositions and the microbial components such as mEVs thereof are generally purified from residual habitat products.

As used herein a “lipid” includes fats, oils, triglycerides, cholesterol, phospholipids, fatty acids in any form including free fatty acids. Fats, oils and fatty acids can be saturated, unsaturated (cis or trans) or partially unsaturated (cis or trans).

The term “LPS mutant or lipopolysaccharide mutant” broadly refers to selected bacteria that comprises loss of LPS. Loss of LPS might be due to mutations or disruption to genes involved in lipid A biosynthesis, such as lpxA, lpxC, and lpxD. Bacteria comprising LPS mutants can be resistant to aminoglycosides and polymyxins (polymyxin B and colistin).

“Metabolite” as used herein refers to any and all molecular compounds, compositions, molecules, ions, co-factors, catalysts or nutrients used as substrates in any cellular or microbial metabolic reaction or resulting as product compounds, compositions, molecules, ions, co-factors, catalysts or nutrients from any cellular or microbial metabolic reaction.

“Microbe” refers to any natural or engineered organism characterized as a archaeaon, parasite, bacterium, fungus, microscopic alga, protozoan, and the stages of development or life cycle stages (e.g., vegetative, spore (including sporulation, dormancy, and germination), latent, biofilm) associated with the organism. Examples of gut microbes include: Actinomyces graevenitzii, Actinomyces odontolyticus, Akkermansia muciniphila, Bacteroides caccae, Bacteroides fragilis, Bacteroides putredinis, Bacteroides thetaiotaomicron, Bacteroides vultagus, Bifidobacterium adolescentis, Bifidobacterium bifidum, Bilophila wadsworthia, Blautia, Butyrivibrio, Campylobacter gracilis, Clostridia cluster III, Clostridia cluster IV, Clostridia cluster IX (Acidaminococcaceae group), Clostridia cluster XI, Clostridia cluster XIII (Peptostreptococcus group), Clostridia cluster XIV, Clostridia cluster XV, Collinsella aerofaciens, Coprococcus, Corynebacterium sunsvallense, Desulfomonas pigra, Dorea formicigenerans, Dorea longicatena, Escherichia coli, Eubacterium hadrum, Eubacterium rectale, Faecalibacteria prausnitzii, Gemella, Lactococcus, Lanchnospira, Mollicutes cluster XVI, Mollicutes cluster XVIII, Prevotella, Rothia mucilaginosa, Ruminococcus callidus, Ruminococcus gnavus, Ruminococcus torques, and Streptococcus.

“Microbial extracellular vesicles” (mEVs) can be obtained from microbes such as bacteria, archaea, fungi, microscopic algae, protozoans, and parasites. In some embodiments, the mEVs are obtained from bacteria. mEVs include secreted microbial extracellular vesicles (smEVs) and processed microbial extracellular vesicles (pmEVs). “Secreted microbial extracellular vesicles” (smEVs) are naturally-produced vesicles derived from microbes. smEVs are comprised of microbial lipids and/or microbial proteins and/or microbial nucleic acids and/or microbial carbohydrate moieties, and are isolated from culture supernatant. The natural production of these vesicles can be artificially enhanced (e.g., increased) or decreased through manipulation of the environment in which the bacterial cells are being cultured (e.g., by media or temperature alterations). Further, smEV compositions may be modified to reduce, increase, add, or remove microbial components or foreign substances to alter efficacy, immune stimulation, stability, immune stimulatory capacity, stability, organ targeting (e.g., lymph node), absorption (e.g., gastrointestinal), and/or yield (e.g., thereby altering the efficacy). As used herein, the term “purified smEV composition” or “smEV composition” refers to a preparation of smEVs that have been separated from at least one associated substance found in a source material (e.g., separated from at least one other microbial component) or any material associated with the smEVs in any process used to produce the preparation. It can also refer to a composition that has been significantly enriched for specific components. “Processed microbial extracellular vesicles” (pmEVs) are a non-naturally-occurring collection of microbial membrane components that have been purified from artificially lysed microbes (e.g., bacteria) (e.g., microbial membrane components that have been separated from other, intracellular microbial cell components), and which may comprise particles of a varied or a selected size range, depending on the method of purification. A pool of pmEVs is obtained by chemically disrupting (e.g., by lysozyme and/or lysostaphin) and/or physically disrupting (e.g., by mechanical force) microbial cells and separating the microbial membrane components from the intracellular components through centrifugation and/or ultracentrifugation, or other methods. The resulting pmEV mixture contains an enrichment of the microbial membranes and the components thereof (e.g., peripherally associated or integral membrane proteins, lipids, glycans, polysaccharides, carbohydrates, other polymers), such that there is an increased concentration of microbial membrane components, and a decreased concentration (e.g., dilution) of intracellular contents, relative to whole microbes. For gram-positive bacteria, pmEVs may include cell or cytoplasmic membranes. For gram-negative bacteria, a pmEV may include inner and outer membranes. pmEVs may be modified to increase purity, to adjust the size of particles in the composition, and/or modified to reduce, increase, add or remove, microbial components or foreign substances to alter efficacy, immune stimulation, stability, immune stimulatory capacity, stability, organ targeting (e.g., lymph node), absorption (e.g., gastrointestinal), and/or yield (e.g., thereby altering the efficacy). pmEVs can be modified by adding, removing, enriching for, or diluting specific components, including intracellular components from the same or other microbes. As used herein, the term “purified pmEV composition” or “pmEV composition” refers to a preparation of pmEVs that have been separated from at least one associated substance found in a source material (e.g., separated from at least one other microbial component) or any material associated with the pmEVs in any process used to produce the preparation. It can also refer to a composition that has been significantly enriched for specific components.

“Microbiome” broadly refers to the microbes residing on or in body site of a subject or patient. Microbes in a microbiome may include bacteria, viruses, eukaryotic microorganisms, and/or viruses. Individual microbes in a microbiome may be metabolically active, dormant, latent, or exist as spores, may exist planktonically or in biofilms, or may be present in the microbiome in sustainable or transient manner. The microbiome may be a commensal or healthy-state microbiome or a disease-state microbiome. The microbiome may be native to the subject or patient, or components of the microbiome may be modulated, introduced, or depleted due to changes in health state (e.g., pre-diseased or diseased state) or treatment conditions (e.g., antibiotic treatment, exposure to different microbes). In some aspects, the microbiome occurs at a mucosal surface. In some aspects, the microbiome is a gut microbiome.

A “microbiome profile” or a “microbiome signature” of a tissue or sample refers to an at least partial characterization of the bacterial makeup of a microbiome. In some embodiments, a microbiome profile indicates whether at least 2, 3, 4, 5, 6, 7, 8, 9, 10, 15, 20, 25, 30, 35, 40, 45, 50, 55, 60, 65, 70, 75, 80, 85, 90, 95, 100 or more bacterial strains are present or absent in a microbiome. In some embodiments, a microbiome profile indicates whether at least 2, 3, 4, 5, 6, 7, 8, 9, 10, 15, 20, 25, 30, 35, 40, 45, 50, 55, 60, 65, 70, 75, 80, 85, 90, 95, 100 or more disease-associated bacterial strains are present in a sample. In some embodiments, the microbiome profile indicates the relative or absolute amount of each bacterial strain detected in the sample. In some embodiments, the microbiome profile is a disease-associated microbiome profile. A disease-associated microbiome profile is a microbiome profile that occurs with greater frequency in a subject who has the disease than in the general population. In some embodiments, the disease-associated microbiome profile comprises a greater number of or amount of disease-associated bacteria than is normally present in a microbiome of an otherwise equivalent tissue or sample taken from an individual who does not have the disease.

“Modified” in reference to a bacteria broadly refers to a bacteria that has undergone a change from its wild-type form. Bacterial modification can result from engineering bacteria. Examples of bacterial modifications include genetic modification, gene expression modification, phenotype modification, formulation modification, chemical modification, and dose or concentration. Examples of improved properties are described throughout this specification and include, e.g., attenuation, auxotrophy, homing, or antigenicity. Phenotype modification might include, by way of example, bacteria growth in media that modify the phenotype of a bacterium such that it increases or decreases virulence.

“Operational taxonomic units” and “OTU(s)” refer to a terminal leaf in a phylogenetic tree and is defined by a nucleic acid sequence, e.g., the entire genome, or a specific genetic sequence, and all sequences that share sequence identity to this nucleic acid sequence at the level of species. In some embodiments the specific genetic sequence may be the 16S sequence or a portion of the 16S sequence. In other embodiments, the entire genomes of two entities are sequenced and compared. In another embodiment, select regions such as multilocus sequence tags (MLST), specific genes, or sets of genes may be genetically compared. For 16S, OTUs that share ≥ 97% average nucleotide identity across the entire 16S or some variable region of the 16S are considered the same OTU. See e.g., Claesson MJ, Wang Q, O′Sullivan O, Greene-Diniz R, Cole JR, Ross RP, and O′Toole PW. 2010. Comparison of two next-generation sequencing technologies for resolving highly complex microbiota composition using tandem variable 16S rRNA gene regions. Nucleic Acids Res 38: e200. Konstantinidis KT, Ramette A, and Tiedje JM. 2006. The bacterial species definition in the genomic era. Philos Trans R Soc Lond B Biol Sci 361: 1929-1940. For complete genomes, MLSTs, specific genes, other than 16S, or sets of genes OTUs that share ≥ 95% average nucleotide identity are considered the same OTU. See e.g., Achtman M, and Wagner M. 2008. Microbial diversity and the genetic nature of microbial species. Nat. Rev. Microbiol. 6: 431-440. Konstantinidis KT, Ramette A, and Tiedje JM. 2006. The bacterial species definition in the genomic era. Philos Trans R Soc Lond B Biol Sci 361: 1929-1940. OTUs are frequently defined by comparing sequences between organisms. Generally, sequences with less than 95% sequence identity are not considered to form part of the same OTU. OTUs may also be characterized by any combination of nucleotide markers or genes, in particular highly conserved genes (e.g., “house-keeping” genes), or a combination thereof. Operational Taxonomic Units (OTUs) with taxonomic assignments made to, e.g., genus, species, and phylogenetic clade are provided herein.

As used herein, a gene is “overexpressed” in a bacteria if it is expressed at a higher level in an engineered bacteria under at least some conditions than it is expressed by a wild-type bacteria of the same species under the same conditions. Similarly, a gene is “underexpressed” in a bacteria if it is expressed at a lower level in an engineered bacteria under at least some conditions than it is expressed by a wild-type bacteria of the same species under the same conditions.

The terms “polynucleotide”, and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function. The following are non-limiting examples of polynucleotides: coding or non-coding regions of a gene or gene fragment, loci (locus) defined from linkage analysis, exons, introns, messenger RNA (mRNA), micro RNA (miRNA), silencing RNA (siRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. A polynucleotide may be further modified, such as by conjugation with a labeling component. In all nucleic acid sequences provided herein, U nucleotides are interchangeable with T nucleotides.

As used herein, a substance is “pure” if it is substantially free of other components. The terms “purify,” “purifying” and “purified” refer to bacteria or an mEV (such as an smEV and/or a pmEV) preparation or other material that has been separated from at least some of the components with which it was associated either when initially produced or generated (e.g., whether in nature or in an experimental setting), or during any time after its initial production. An mEV (such as an smEV and/or a pmEV) preparation or compositions may be considered purified if it is isolated at or after production, such as from one or more other bacterial components, and a purified microbe or microbial population may contain other materials up to about 10%, about 20%, about 30%, about 40%, about 50%, about 60%, about 70%, about 80%, about 90%, or above about 90% and still be considered “purified.” In some embodiments, purified mEVs (such as smEVs and/or pmEVs) are more than about 80%, about 85%, about 90%, about 91%, about 92%, about 93%, about 94%, about 95%, about 96%, about 97%, about 98%, about 99%, or more than about 99% pure. mEV (such as an smEV and/or a pmEV) compositions (or preparations) are, e.g., purified from residual habitat products.

As used herein, the term “purified mEV composition” or “mEV composition” refers to a preparation that includes mEVs (such as smEVs and/or pmEVs) that have been separated from at least one associated substance found in a source material (e.g., separated from at least one other bacterial component) or any material associated with the mEVs (such as smEVs and/or pmEVs) in any process used to produce the preparation. It also refers to a composition that has been significantly enriched or concentrated. In some embodiments, the mEVs (such as smEVs and/or pmEVs) are concentrated by 2 fold, 3-fold, 4-fold, 5-fold, 10-fold, 100-fold, 1000-fold, 10,000-fold or more than 10,000 fold.

“Residual habitat products” refers to material derived from the habitat for microbiota within or on a subject. For example, fermentation cultures of microbes can contain contaminants, e.g., other microbe strains or forms (e.g., bacteria, virus, mycoplasm, and/or fungus). For example, microbes live in feces in the gastrointestinal tract, on the skin itself, in saliva, mucus of the respiratory tract, or secretions of the genitourinary tract (i.e., biological matter associated with the microbial community). Substantially free of residual habitat products means that the microbial composition no longer contains the biological matter associated with the microbial environment on or in the culture or human or animal subject and is 100% free, 99% free, 98% free, 97% free, 96% free, or 95% free of any contaminating biological matter associated with the microbial community. Residual habitat products can include abiotic materials (including undigested food) or it can include unwanted microorganisms. Substantially free of residual habitat products may also mean that the microbial composition contains no detectable cells from a culture contaminant or a human or animal and that only microbial cells are detectable. In one embodiment, substantially free of residual habitat products may also mean that the microbial composition contains no detectable viral (including bacteria, viruses (e.g., phage)), fungal, mycoplasmal contaminants. In another embodiment, it means that fewer than 1×10⁻²%, 1×10⁻³%, 1×10⁻⁴%, 1×10⁻⁵%, 1×10⁻⁶%, 1×10⁻⁷%, 1×10⁻⁸% of the viable cells in the microbial composition are human or animal, as compared to microbial cells. There are multiple ways to accomplish this degree of purity, none of which are limiting. Thus, contamination may be reduced by isolating desired constituents through multiple steps of streaking to single colonies on solid media until replicate (such as, but not limited to, two) streaks from serial single colonies have shown only a single colony morphology. Alternatively, reduction of contamination can be accomplished by multiple rounds of serial dilutions to single desired cells (e.g., a dilution of 10⁻⁸ or 10⁻⁹), such as through multiple 10-fold serial dilutions. This can further be confirmed by showing that multiple isolated colonies have similar cell shapes and Gram staining behavior. Other methods for confirming adequate purity include genetic analysis (e.g., PCR, DNA sequencing), serology and antigen analysis, enzymatic and metabolic analysis, and methods using instrumentation such as flow cytometry with reagents that distinguish desired constituents from contaminants.

As used herein, “specific binding” refers to the ability of an antibody to bind to a predetermined antigen or the ability of a polypeptide to bind to its predetermined binding partner. Typically, an antibody or polypeptide specifically binds to its predetermined antigen or binding partner with an affinity corresponding to a K_{D} of about 10⁻⁷ M or less, and binds to the predetermined antigen/binding partner with an affinity (as expressed by K_{D}) that is at least 10 fold less, at least 100 fold less or at least 1000 fold less than its affinity for binding to a non-specific and unrelated antigen/binding partner (e.g., BSA, casein). Alternatively, specific binding applies more broadly to a two component system where one component is a protein, lipid, or carbohydrate or combination thereof and engages with the second component which is a protein, lipid, carbohydrate or combination thereof in a specific way.

“Strain” refers to a member of a bacterial species with a genetic signature such that it may be differentiated from closely-related members of the same bacterial species. The genetic signature may be the absence of all or part of at least one gene, the absence of all or part of at least on regulatory region (e.g., a promoter, a terminator, a riboswitch, a ribosome binding site), the absence (“curing”) of at least one native plasmid, the presence of at least one recombinant gene, the presence of at least one mutated gene, the presence of at least one foreign gene (a gene derived from another species), the presence at least one mutated regulatory region (e.g., a promoter, a terminator, a riboswitch, a ribosome binding site), the presence of at least one non-native plasmid, the presence of at least one antibiotic resistance cassette, or a combination thereof. Genetic signatures between different strains may be identified by PCR amplification optionally followed by DNA sequencing of the genomic region(s) of interest or of the whole genome. In the case in which one strain (compared with another of the same species) has gained or lost antibiotic resistance or gained or lost a biosynthetic capability (such as an auxotrophic strain), strains may be differentiated by selection or counter-selection using an antibiotic or nutrient/metabolite, respectively.

The terms “subject” or “patient” refers to any mammal. A subject or a patient described as “in need thereof” refers to one in need of a treatment (or prevention) for a disease. Mammals (i.e., mammalian animals) include humans, laboratory animals (e.g., primates, rats, mice), livestock (e.g., cows, sheep, goats, pigs), and household pets (e.g., dogs, cats, rodents). The subject may be a human. The subject may be a non-human mammal including but not limited to of a dog, a cat, a cow, a horse, a pig, a donkey, a goat, a camel, a mouse, a rat, a guinea pig, a sheep, a llama, a monkey, a gorilla or a chimpanzee. The subject may be healthy, or may be suffering from a disease (e.g., a neuroinflammatory disease, a neurodegenerative disease, a neuromuscular disease, and/or a psychiatric disorder) at any developmental stage, wherein any of the stages are either caused by or opportunistically supported of a disease associated or causative pathogen, or may be at risk of developing a disease, or transmitting to others a disease associated or disease causative pathogen. In some embodiments, a subject has a neuroinflammatory disease, a neurodegenerative disease, a neuromuscular disease, and/or a psychiatric disorder. In some embodiments, the subject has undergone a therapy to treat the disease.

As used herein, the term “treating” a disease in a subject or “treating” a subject having or suspected of having a disease refers to administering to the subject to a pharmaceutical treatment, e.g., the administration of one or more agents, such that at least one symptom of the disease is decreased or prevented from worsening. Thus, in one embodiment, “treating” refers inter alia to delaying progression, expediting remission, inducing remission, augmenting remission, speeding recovery, increasing efficacy of or decreasing resistance to alternative therapeutics, or a combination thereof. As used herein, the term “preventing” a disease in a subject refers to administering to the subject to a pharmaceutical treatment, e.g., the administration of one or more agents, such that onset of at least one symptom of the disease is delayed or prevented.

As used herein, a “type” of bacteria may be distinguished from other bacteria by: genus, species, sub-species, strain or by any other taxonomic categorization, whether based on morphology, physiology, genotype, protein expression or other characteristics known in the art.

### Bacteria

In certain aspects, provided herein are pharmaceutical compositions that comprise mEVs (such as smEVs and/or pmEVs) from Veillonella parvula (e.g., Veillonella parvula Strain A) bacteria, Veillonella parvula (e.g., Veillonella parvula Strain A) bacteria, or any combination thereof.

In some embodiments, the Veillonella parvula bacteria is Veillonella parvula bacteria Strain A (ATCC Deposit Number PTA-125691) (also referred to as Veillonella parvula Strain A or Strain A). In some embodiments, the Strain A bacteria are gamma irradiated (G.I.) (e.g., G.I. Strain A) (e.g., gamma irradiated at 17.5 or 25 kGy).

In some embodiments, the bacteria is a strain comprising at least 90%, at least 91%, at least 92%, at least 93%, at least 94%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic, 16S or CRISPR nucleotide sequence) of Veillonella parvula Strain A (ATCC Deposit Number PTA-125691).

Under the terms of the Budapest Treaty on the International Recognition of the Deposit of Microorganisms for the Purpose of Patent Procedure, Veillonella parvula Strain A was deposited on Jan. 25, 2019, with the American Type Culture Collection (ATCC) of 10801 University Boulevard, Manassas, Va. 20110-2209 USA and were assigned ATCC Accession Numbers PTA-125691.

Applicant represents that the ATCC is a depository affording permanence of the deposit and ready accessibility thereto by the public if a patent is granted. All restrictions on the availability to the public of the material so deposited will be irrevocably removed upon the granting of a patent. The material will be available during the pendency of the patent application to one determined by the Commissioner to be entitled thereto under 37 CFR 1.14 and 35 U.S.C. 122. The deposited material will be maintained with all the care necessary to keep it viable and uncontaminated for a period of at least five years after the most recent request for the furnishing of a sample of the deposited plasmid, and in any case, for a period of at least thirty (30) years after the date of deposit or for the enforceable life of the patent, whichever period is longer. Applicant acknowledges its duty to replace the deposit should the depository be unable to furnish a sample when requested due to the condition of the deposit.

The Veillonella bacteria (e.g., Veillonella parvula Strain A (ATCC Deposit Number PTA-125691)) can be cultured according to methods known in the art. For example, the Veillonella bacteria (e.g., Veillonella parvula Strain A (ATCC Deposit Number PTA-125691)) can be grown in ATCC Medium 2722, ATCC Medium 1490, or other medium using methods disclosed, for example in Caballero et al., 2017. “Cooperating Commensals Restore Colonization Resistance to Vancomycin-Resistant Enterococcus faecium” Cell Host & Microbe 21:592-602, which is hereby incorporated by reference in its entirety.

In some embodiments, the mEVs (such as smEVs and/or pmEVs), bacteria, or any combination thereof, described herein are obtained from a strain of Veillonella parvula bacteria comprising a genomic sequence that is at least 90%, at least 91%, at least 92%, at least 93%, at least 94%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the genomic sequence of the strain of bacteria deposited with the ATCC Deposit number PTA-125691. In some embodiments, the mEVs (such as smEVs and/or pmEVs), bacteria, or any combination thereof, described herein are obtained from a strain of Veillonella parvula bacteria comprising a 16S sequence that is at least 90%, at least 91%, at least 92%, at least 93%, at least 94%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the 16S sequence of the strain of bacteria deposited with the ATCC Deposit number PTA-125691.

In some embodiments, the mEVs (such as smEVs and/or pmEVs), bacteria, or any combination thereof, of the pharmaceutical compositions described herein are lyophilized.

In some embodiments, the mEVs (such as smEVs and/or pmEVs), bacteria, or any combination thereof, of the pharmaceutical compositions described herein are gamma irradiated (e.g., at 17.5 or 25 kGy).

In some embodiments, the mEVs (such as smEVs and/or pmEVs), bacteria, or any combination thereof, of the pharmaceutical compositions described herein are UV irradiated.

In some embodiments, the mEVs (such as smEVs and/or pmEVs), bacteria, or any combination thereof, of the pharmaceutical compositions described herein are heat inactivated (e.g., at 50° C. for two hours or at 90° C. for two hours).

In some embodiments, the mEVs (such as smEVs and/or pmEVs), bacteria, or any combination thereof, of the pharmaceutical compositions described herein are acid treated.

In some embodiments, the mEVs (such as smEVs and/or pmEVs), bacteria, or any combination thereof, of the pharmaceutical compositions described herein are oxygen sparged (e.g., at 0.1 vvm for two hours).

The phase of growth can affect the amount or properties of bacteria and/or mEVs (such as smEVs and/or pmEVs) produced by bacteria. For example, in the methods of bacteria preparation provided herein, bacteria can be isolated, e.g., from a culture, at the start of the log phase of growth, midway through the log phase, and/or once stationary phase growth has been reached. As another example, in the methods of preparing mEVs (such as smEVs and/or pmEVs) provided herein, mEVs (such as smEVs and/or pmEVs) can be prepared from a culture, at the start of the log phase of growth, midway through the log phase, and/or once stationary phase growth has been reached.

### Modified mEVs

In some aspects, the mEVs (such as smEVs and/or pmEVs) described herein are modified such that they comprise, are linked to, and/or are bound by a therapeutic moiety.

In some embodiments, the therapeutic moiety is a target-specific moiety. In some embodiments, the target-specific moiety has binding specificity for a target cell (e.g., has binding specificity for a target cell-specific antigen). In some embodiments, the target-specific moiety comprises an antibody or antigen binding fragment thereof. In some embodiments, the target-specific moiety comprises a ligand for a receptor expressed on the surface of a target cell or a receptor-binding fragment thereof. In some embodiments, the target-specific moiety is a bipartite fusion protein that has two parts: a first part that binds to and/or is linked to the bacterium and a second part that is capable of binding to a target cell (e.g., by having binding specificity for a target-specific antigen). In some embodiments, the first part is a fragment of or a full-length peptidoglycan recognition protein, such as PGRP. In some embodiments the first part has binding specificity for the mEV (e.g., by having binding specificity for a bacterial antigen). In some embodiments, the first and/or second part comprises an antibody or antigen binding fragment thereof. In some embodiments, the first and/or second part comprises a ligand for a receptor expressed on the surface of a target cell or a receptor-binding fragment thereof. In certain embodiments, co-administration of the target-specific moiety with the mEVs (either in combination or in separate administrations) increases the targeting of the mEVs to the target cells.

In some embodiments, the mEVs described herein are modified such that they comprise, are linked to, and/or are bound by a magnetic and/or paramagnetic moiety (e.g., a magnetic bead). In some embodiments, the magnetic and/or paramagnetic moiety is comprised by and/or directly linked to the bacteria. In some embodiments, the magnetic and/or paramagnetic moiety is linked to and/or a part of an mEV-binding moiety that that binds to the mEV. In some embodiments, the mEV-binding moiety is a fragment of or a full-length peptidoglycan recognition protein, such as PGRP. In some embodiments the mEV-binding moiety has binding specificity for the mEV (e.g., by having binding specificity for a bacterial antigen). In some embodiments, the mEV-binding moiety comprises an antibody or antigen binding fragment thereof. In some embodiments, the mEV-binding moiety comprises a T cell receptor. In some embodiments, the mEV-binding moiety comprises a ligand for a receptor expressed on the surface of a cell or a receptor-binding fragment thereof. In certain embodiments, co-administration of the magnetic and/or paramagnetic moiety with the mEVs (either together or in separate administrations) can be used to increase the targeting of the mEVs (e.g., to cells and/or a part of a subject where the target cells are present).

### Production of Processed Microbial Extracellular Vesicles (pmEVs)

In certain aspects, the pmEVs described herein can be prepared using any method known in the art.

In some embodiments, the pmEVs are prepared without a pmEV purification step. For example, in some embodiments, Veillonella parvula (e.g., Veillonella parvula Strain A) bacteria from which the pmEVs described herein are released are killed using a method that leaves the Veillonella parvula bacterial pmEVs intact, and the resulting Veillonella parvula bacterial components, including the pmEVs, are used in the methods and compositions described herein. In some embodiments, the Veillonella parvula (e.g., Veillonella parvula Strain A) bacteria are killed using an antibiotic (e.g., using an antibiotic described herein). In some embodiments, the Veillonella parvula (e.g., Veillonella parvula Strain A) bacteria are killed using UV irradiation.

In some embodiments, the pmEVs described herein are purified from one or more other Veillonella parvula bacterial components. Methods for purifying pmEVs from Veillonella parvula (e.g., Veillonella parvula Strain A) bacteria (and optionally, other bacterial components) are known in the art. In some embodiments, pmEVs are prepared from Veillonella parvula bacterial cultures using methods described in Thein, et al. (J. Proteome Res. 9(12):6135-6147 (2010)) or Sandrini, et al. (Bio-protocol 4(21): e1287 (2014)), each of which is hereby incorporated by reference in its entirety. In some embodiments, the bacteria are cultured to high optical density and then centrifuged to pellet bacteria (e.g., at 10,000- 15,000 × g for 10- 15 min at room temperature or 4° C.). In some embodiments, the supernatants are discarded and cell pellets are frozen at -80° C. In some embodiments, cell pellets are thawed on ice and resuspended in 100 mM Tris-HCl, pH 7.5 supplemented with 1 mg/mL DNase I. In some embodiments, cells are lysed using an Emulsiflex C-3 (Avestin, Inc.) under conditions recommended by the manufacturer. In some embodiments, debris and unlysed cells are pelleted by centrifugation at 10,000 × g for 15 min at 4° C. In some embodiments, supernatants are then centrifuged at 120,000 × g for 1 hour at 4° C. In some embodiments, pellets are resuspended in ice-cold 100 mM sodium carbonate, pH 11, incubated with agitation for 1 hr at 4° C., and then centrifuged at 120,000 × g for 1 hour at 4° C. In some embodiments, pellets are resuspended in 100 mM Tris-HCl, pH 7.5, re-centrifuged at 120,000 × g for 20 min at 4° C., and then resuspended in 0.1 M Tris-HCl, pH 7.5 or in PBS. In some embodiments, samples are stored at -20° C.

In certain aspects, pmEVs are obtained by methods adapted from Sandrini et al, 2014. In some embodiments, Veillonella parvula (e.g., Veillonella parvula Strain A) bacterial cultures are centrifuged at 10,000-15,500 × g for 10-15 min at room temp or at 4° C. In some embodiments, cell pellets are frozen at -80° C. and supernatants are discarded. In some embodiments, cell pellets are thawed on ice and resuspended in 10 mM Tris-HCl, pH 8.0, 1 mM EDTA supplemented with 0.1 mg/mL lysozyme. In some embodiments, samples are incubated with mixing at room temp or at 37° C. for 30 min. In some embodiments, samples are re-frozen at -80° C. and thawed again on ice. In some embodiments, DNase I is added to a final concentration of 1.6 mg/mL and MgCl2 to a final concentration of 100 mM. In some embodiments, samples are sonicated using a QSonica Q500 sonicator with 7 cycles of 30 sec on and 30 sec off. In some embodiments, debris and unlysed cells are pelleted by centrifugation at 10,000 × g for 15 min. at 4° C. In some embodiments, supernatants are then centrifuged at 110,000 × g for 15 min at 4° C. In some embodiments, pellets are resuspended in 10 mM Tris-HCl, pH 8.0, 2% Triton X-100 and incubated 30-60 min with mixing at room temperature. In some embodiments, samples are centrifuged at 110,000 × g for 15 min at 4° C. In some embodiments, pellets are resuspended in PBS and stored at -20° C.

In certain aspects, a method of forming (e.g., preparing) isolated Veillonella parvula (e.g., Veillonella parvula Strain A) bacterial pmEVs, described herein, comprises the steps of: (a) centrifuging a Veillonella parvula (e.g., Veillonella parvula Strain A) bacterial culture, thereby forming a first pellet and a first supernatant, wherein the first pellet comprises cells; (b) discarding the first supernatant;(c) resuspending the first pellet in a solution; (d) lysing the cells; (e) centrifuging the lysed cells, thereby forming a second pellet and a second supernatant; (f) discarding the second pellet and centrifuging the second supernatant, thereby forming a third pellet and a third supernatant; (g) discarding the third supernatant and resuspending the third pellet in a second solution, thereby forming the isolated Veillonella parvula (e.g., Veillonella parvula Strain A) bacterial pmEVs.

In some embodiments, the method further comprises the steps of: (h) centrifuging the solution of step (g), thereby forming a fourth pellet and a fourth supernatant; (i) discarding the fourth supernatant and resuspending the fourth pellet in a third solution. In some embodiments, the method further comprises the steps of: (j) centrifuging the solution of step (i), thereby forming a fifth pellet and a fifth supernatant; and (k) discarding the fifth supernatant and resuspending the fifth pellet in a fourth solution.

In some embodiments, the centrifugation of step (a) is at 10,000 × g. In some embodiments the centrifugation of step (a) is for 10-15 minutes. In some embodiments, the centrifugation of step (a) is at 4° C. or room temperature. In some embodiments, step (b) further comprises freezing the first pellet at -80° C. . In some embodiments, the solution in step (c) is 100 mM Tris-HCl, pH 7.5 supplemented with 1 mg/ml DNaseI. In some embodiments, the solution in step (c) is 10 mM Tris-HCl, pH 8.0, 1 mM EDTA, supplemented with 0.1 mg/ml lysozyme. In some embodiments, step (c) further comprises incubating for 30 minutes at 37° C. or room temperature. In some embodiments, step (c) further comprises freezing the first pellet at -80° C. . In some embodiments, step (c) further comprises adding DNase I to a final concentration of 1.6 mg/ml. In some embodiments, step (c) further comprises adding MgCl₂ to a final concentration of 100 mM. In some embodiments, the cells are lysed in step (d) via homogenization. In some embodiments, the cells are lysed in step (d) via emulsiflex C3. In some embodiments, the cells are lysed in step (d) via sonication. In some embodiments, the cells are sonicated in 7 cycles, wherein each cycle comprises 30 seconds of sonication and 30 seconds without sonication. In some embodiments, the centrifugation of step (e) is at 10,000 × g. In some embodiments, the centrifugation of step (e) is for 15 minutes. In some embodiments, the centrifugation of step (e) is at 4° C. or room temperature.

In some embodiments, the centrifugation of step (f) is at 120,000 × g. In some embodiments, the centrifugation of step (f) is at 110,000 × g. In some embodiments, the centrifugation of step (f) is for 1 hour. In some embodiments, the centrifugation of step (f) is for 15 minutes. In some embodiments, the centrifugation of step (f) is at 4° C. or room temperature. In some embodiments, the second solution in step (g) is 100 mM sodium carbonate, pH 11. In some embodiments, the second solution in step (g) is 10 mM Tris-HCl pH 8.0, 2% triton X-100. In some embodiments, step (g) further comprises incubating the solution for 1 hour at 4° C. In some embodiments, step (g) further comprises incubating the solution for 30-60 minutes at room temperature. In some embodiments, the centrifugation of step (h) is at 120,000 × g. In some embodiments, the centrifugation of step (h) is at 110,000 × g. In some embodiments, the centrifugation of step (h) is for 1 hour. In some embodiments, the centrifugation of step (h) is for 15 minutes. In some embodiments, the centrifugation of step (h) is at 4° C. or room temperature. In some embodiments, the third solution in step (i) is 100 mM Tris-HCl, pH 7.5. In some embodiments, the third solution in step (i) is PBS. In some embodiments, the centrifugation of step (j) is at 120,000 × g. In some embodiments, the centrifugation of step (j) is for 20 minutes. In some embodiments, the centrifugation of step (j) is at 4° C. or room temperature. In some embodiments, the fourth solution in step (k) is 100 mM Tris-HCl, pH 7.5 or PBS.

pmEVs obtained by methods provided herein may be further purified by size based column chromatography, by affinity chromatography, and by gradient ultracentrifugation, using methods that may include, but are not limited to, use of a sucrose gradient or Optiprep gradient. Briefly, using a sucrose gradient method, if ammonium sulfate precipitation or ultracentrifugation were used to concentrate the filtered supernatants, pellets are resuspended in 60% sucrose, 30 mM Tris, pH 8.0. If filtration was used to concentrate the filtered supernatant, the concentrate is buffer exchanged into 60% sucrose, 30 mM Tris, pH 8.0, using an Amicon Ultra column. Samples are applied to a 35-60% discontinuous sucrose gradient and centrifuged at 200,000 × g for 3-24 hours at 4° C. Briefly, using an Optiprep gradient method, if ammonium sulfate precipitation or ultracentrifugation were used to concentrate the filtered supernatants, pellets are resuspended in 35% Optiprep in PBS. In some embodiments, if filtration was used to concentrate the filtered supernatant, the concentrate is diluted using 60% Optiprep to a final concentration of 35% Optiprep. Samples are applied to a 35-60% discontinuous sucrose gradient and centrifuged at 200,000 × g for 3-24 hours at 4° C.

In some embodiments, to confirm sterility and isolation of the pmEV preparations, pmEVs are serially diluted onto agar medium used for routine culture of the bacteria being tested, and incubated using routine conditions. Non-sterile preparations are passed through a 0.22 um filter to exclude intact cells. To further increase purity, isolated pmEVs may be DNase or proteinase K treated.

In some embodiments, the sterility of the pmEV preparations can be confirmed by plating a portion of the pmEVs onto agar medium used for standard culture of the bacteria used in the generation of the pmEVs and incubating using standard conditions.

In some embodiments select pmEVs are isolated and enriched by chromatography and binding surface moieties on pmEVs. In other embodiments, select pmEVs are isolated and/or enriched by fluorescent cell sorting by methods using affinity reagents, chemical dyes, recombinant proteins or other methods known to one skilled in the art.

The pmEVs can be analyzed, e.g., as described in Jeppesen, et al. Cell 177:428 (2019).

In some embodiments, pmEVs are lyophilized. In some embodiments, pmEVs are gamma irradiated (e.g., at 17.5 or 25 kGy). In some embodiments, pmEVs are UV irradiated. In some embodiments, pmEVs are heat inactivated (e.g., at 50° C. for two hours or at 90° C. for two hours). In some embodiments, pmEVs are acid treated. In some embodiments, pmEVs are oxygen sparged (e.g., at 0.1 vvm for two hours).

The phase of growth can affect the amount or properties of bacteria. In the methods of pmEV preparation provided herein, pmEVs can be isolated, e.g., from a culture, at the start of the log phase of growth, midway through the log phase, and/or once stationary phase growth has been reached.

### Production of Secreted Microbial Extracellular Vesicles (smEVs)

In certain aspects, the smEVs described herein can be prepared using any method known in the art.

In some embodiments, the smEVs are prepared without an smEV purification step. For example, in some embodiments, bacteria described herein are killed using a method that leaves the smEVs intact and the resulting Veillonella parvula (e.g., Veillonella parvula Strain A) bacterial components, including the smEVs, are used in the methods and compositions described herein. In some embodiments, the Veillonella parvula (e.g., Veillonella parvula Strain A) bacteria are killed using an antibiotic (e.g., using an antibiotic described herein). In some embodiments, the Veillonella parvula (e.g., Veillonella parvula Strain A) bacteria are killed using UV irradiation. In some embodiments, the Veillonella parvula (e.g., Veillonella parvula Strain A) bacteria are heat-killed.

In some embodiments, the smEVs described herein are purified from one or more other Veillonella parvula (e.g., Veillonella parvula Strain A) bacterial components. Methods for purifying smEVs from bacteria are known in the art. In some embodiments, smEVs are prepared from Veillonella parvula (e.g., Veillonella parvula Strain A) bacterial cultures using methods described in S. Bin Park, et al. PLoS ONE. 6(3):e17629 (2011) or G. Norheim, et al. PLoS ONE. 10(9): e0134353 (2015) or Jeppesen, et al. Cell 177:428 (2019), each of which is hereby incorporated by reference in its entirety. In some embodiments, the Veillonella parvula (e.g., Veillonella parvula Strain A) bacteria are cultured to high optical density and then centrifuged to pellet Veillonella parvula bacteria (e.g., at 10,000 × g for 30 min at 4° C., at 15,500 × g for 15 min at 4° C.). In some embodiments, the culture supernatants are then passed through filters to exclude intact bacterial cells (e.g., a 0.22 µm filter). In some embodiments, the supernatants are then subjected to tangential flow filtration, during which the supernatant is concentrated, species smaller than 100 kDa are removed, and the media is partially exchanged with PBS. In some embodiments, filtered supernatants are centrifuged to pellet bacterial smEVs (e.g., at 100,000-150,000 × g for 1-3 hours at 4° C., at 200,000 × g for 1-3 hours at 4° C.). In some embodiments, the smEVs are further purified by resuspending the resulting smEV pellets (e.g., in PBS), and applying the resuspended smEVs to an Optiprep (iodixanol) gradient or gradient (e.g., a 30-60% discontinuous gradient, a 0-45% discontinuous gradient), followed by centrifugation (e.g., at 200,000 × g for 4-20 hours at 4° C.). smEV bands can be collected, diluted with PBS, and centrifuged to pellet the smEVs (e.g., at 150,000 × g for 3 hours at 4° C., at 200,000 × g for 1 hour at 4° C.). The purified smEVs can be stored, for example, at -80° C. or -20° C. until use. In some embodiments, the smEVs are further purified by treatment with DNase and/or proteinase K.

For example, in some embodiments, cultures of Veillonella parvula (e.g., Veillonella parvula Strain A) bacteria can be centrifuged at 11,000 × g for 20-40 min at 4° C. to pellet bacteria. Culture supernatants may be passed through a 0.22 µm filter to exclude intact bacterial cells. Filtered supernatants may then be concentrated using methods that may include, but are not limited to, ammonium sulfate precipitation, ultracentrifugation, or filtration. For example, for ammonium sulfate precipitation, 1.5-3 M ammonium sulfate can be added to filtered supernatant slowly, while stirring at 4° C. Precipitations can be incubated at 4° C. for 8-48 hours and then centrifuged at 11,000 × g for 20-40 min at 4° C. The resulting pellets contain bacteria smEVs and other debris. Using ultracentrifugation, filtered supernatants can be centrifuged at 100,000-200,000 × g for 1-16 hours at 4° C. The pellet of this centrifugation contains bacteria smEVs and other debris such as large protein complexes. In some embodiments, using a filtration technique, such as through the use of an Amicon Ultra spin filter or by tangential flow filtration, supernatants can be filtered so as to retain species of molecular weight > 50 or 100 kDa.

Alternatively, smEVs can be obtained from Veillonella parvula (e.g., Veillonella parvula Strain A) bacteria cultures continuously during growth, or at selected time points during growth, for example, by connecting a bioreactor to an alternating tangential flow (ATF) system (e.g., XCell ATF from Repligen). The ATF system retains intact cells (>0.22 um) in the bioreactor, and allows smaller components (e.g., smEVs, free proteins) to pass through a filter for collection. For example, the system may be configured so that the <0.22 um filtrate is then passed through a second filter of 100 kDa, allowing species such as smEVs between 0.22 um and 100 kDa to be collected, and species smaller than 100 kDa to be pumped back into the bioreactor. Alternatively, the system may be configured to allow for medium in the bioreactor to be replenished and/or modified during growth of the culture. smEVs collected by this method may be further purified and/or concentrated by ultracentrifugation or filtration as described above for filtered supernatants.

smEVs obtained by methods provided herein may be further purified by size-based column chromatography, by affinity chromatography, by ion-exchange chromatography, and by gradient ultracentrifugation, using methods that may include, but are not limited to, use of a sucrose gradient or Optiprep gradient. Briefly, using a sucrose gradient method, if ammonium sulfate precipitation or ultracentrifugation were used to concentrate the filtered supernatants, pellets are resuspended in 60% sucrose, 30 mM Tris, pH 8.0. If filtration was used to concentrate the filtered supernatant, the concentrate is buffer exchanged into 60% sucrose, 30 mM Tris, pH 8.0, using an Amicon Ultra column. Samples are applied to a 35-60% discontinuous sucrose gradient and centrifuged at 200,000 × g for 3-24 hours at 4° C. Briefly, using an Optiprep gradient method, if ammonium sulfate precipitation or ultracentrifugation were used to concentrate the filtered supernatants, pellets are resuspended in PBS and 3 volumes of 60% Optiprep are added to the sample. In some embodiments, if filtration was used to concentrate the filtered supernatant, the concentrate is diluted using 60% Optiprep to a final concentration of 35% Optiprep. Samples are applied to a 0-45% discontinuous Optiprep gradient and centrifuged at 200,000 × g for 3-24 hours at 4° C., e.g., 4-24 hours at 4° C.

In some embodiments, to confirm sterility and isolation of the smEV preparations, smEVs are serially diluted onto agar medium used for routine culture of the bacteria being tested, and incubated using routine conditions. Non-sterile preparations are passed through a 0.22 um filter to exclude intact cells. To further increase purity, isolated smEVs may be DNase or proteinase K treated.

In some embodiments, for preparation of smEVs used for in vivo injections, purified smEVs are processed as described previously (G. Norheim, et al. PLoS ONE. 10(9): e0134353 (2015)). Briefly, after sucrose gradient centrifugation, bands containing smEVs are resuspended to a final concentration of 50 µg/mL in a solution containing 3% sucrose or other solution suitable for in vivo injection known to one skilled in the art. This solution may also contain adjuvant, for example aluminum hydroxide at a concentration of 0-0.5% (w/v). In some embodiments, for preparation of smEVs used for in vivo injections, smEVs in PBS are sterile-filtered to < 0.22 um.

In certain embodiments, to make samples compatible with further testing (e.g., to remove sucrose prior to TEM imaging or in vitro assays), samples are buffer exchanged into PBS or 30 mM Tris, pH 8.0 using filtration (e.g., Amicon Ultra columns), dialysis, or ultracentrifugation (200,000 × g, ≥ 3 hours, 4° C.) and resuspension.

In some embodiments, the sterility of the smEV preparations can be confirmed by plating a portion of the smEVs onto agar medium used for standard culture of the bacteria used in the generation of the smEVs and incubating using standard conditions.

In some embodiments, select smEVs are isolated and enriched by chromatography and binding surface moieties on smEVs. In other embodiments, select smEVs are isolated and/or enriched by fluorescent cell sorting by methods using affinity reagents, chemical dyes, recombinant proteins or other methods known to one skilled in the art.

The smEVs can be analyzed, e.g., as described in Jeppesen, et al. Cell 177:428 (2019).

In some embodiments, smEVs are lyophilized. In some embodiments, smEVs are gamma irradiated (e.g., at 17.5 or 25 kGy). In some embodiments, smEVs are UV irradiated. In some embodiments, smEVs are heat inactivated (e.g., at 50° C. for two hours or at 90° C. for two hours). In some embodiments, smEVs s are acid treated. In some embodiments, smEVs are oxygen sparged (e.g., at 0.1 vvm for two hours).

The phase of growth can affect the amount or properties of Veillonella parvula (e.g., Veillonella parvula Strain A) bacteria and/or smEVs produced by Veillonella parvula (e.g., Veillonella parvula Strain A) bacteria. For example, in the methods of smEV preparation provided herein, smEVs can be isolated, e.g., from a culture, at the start of the log phase of growth, midway through the log phase, and/or once stationary phase growth has been reached.

The growth environment (e.g., culture conditions) can affect the amount of smEVs produced by Veillonella parvula (e.g., Veillonella parvula Strain A) bacteria. For example, the yield of smEVs can be increased by an smEV inducer, as provided in Table 2.

TABLE 2

Culture Techniques to Increase smEV Production

| | | |
|---|---|---|
| smEV inducement | smEV inducer | Acts on |
| Temperature | | |
| | Heat RT to 37° C. temp change 37 to 40° C. temp change | stress response simulates infection febrile infection |
| ROS | | |
| | Plumbagin Cumene hydroperoxide Hydrogen Peroxide | oxidative stress response oxidative stress response oxidative stress response |
| Antibiotics | | |
| | Ciprofloxacin Gentamycin Polymyxin B D-cylcloserine | bacterial SOS response protein synthesis outer membrane cell wall |
| Osmolyte | | |
| | NaCl | osmotic stress |
| Metal Ion Stress | | |
| | Iron Chelation EDTA Low Hemin | iron levels removes divalent cations iron levels |
| Media additives or removal | | |
| | Lactate Amino acid deprivation Hexadecane Glucose Sodium bicarbonate PQS | growth stress stress growth ToxT induction vesiculator (from bacteria) membrane anchoring (negativicutes only) enhanced growth |
| | Diamines+ DFMO High nutrients Low nutrients | |
| Other mechanisms | Oxygen No Cysteine Inducing biofilm or floculation Diauxic Growth Phage Urea | oxygen stress in anaerobe oxygen stress in anaerobe |

In the methods of smEVs preparation provided herein, the method can optionally include exposing a culture of Veillonella parvula (e.g., Veillonella parvula Strain A) bacteria to an smEV inducer prior to isolating smEVs from the bacterial culture. The culture of Veillonella parvula (e.g., Veillonella parvula Strain A) bacteria can be exposed to an smEV inducer at the start of the log phase of growth, midway through the log phase, and/or once stationary phase growth has been reached.

### Pharmaceutical Compositions

In certain aspects, provided herein are pharmaceutical compositions that comprise mEVs (such as smEVs and/or pmEVs), bacteria, or any combination thereof, obtained from Veillonella parvula (e.g., Veillonella parvula Strain A) bacteria.

In certain aspects, provided herein are pharmaceutical compositions comprising Veillonella parvula (e.g., Veillonella parvula Strain A) described herein and a pharmaceutically acceptable carrier.

In some embodiments, the pharmaceutical composition comprises about 1 × 10⁵, 5 × 10⁵, 1 × 10⁶, 2 × 10⁶, 3 × 10⁶, 4 × 10⁶, 5 × 10⁶, 6 × 10⁶, 7 × 10⁶, 8 × 10⁶, 9 × 10⁶, 1 × 10⁷, 2 × 10⁷, 3 × 10⁷, 4 × 10⁷, 5 × 10⁷, 6 × 10⁷, 7× 10⁷, 8 × 10⁷, 9 × 10⁷, 1 × 10⁸, 2 × 10⁸, 3 × 10⁸, 4 × 10⁸, 5 × 10⁸, 6 × 10⁸, 7 × 10⁸, 8 × 10⁸, 9 × 10⁸ or 1 × 10⁹, 1 × 10¹⁰, 2×10¹⁰, 2.1×10¹⁰, 2.2×10¹⁰, 2.3×10¹⁰, 2.4×10¹⁰, 2.5×10¹⁰, 2.6×10¹⁰, 2.7×10¹⁰, 2.8×10¹⁰, 2.9×10¹⁰, 3×10¹⁰, 3.1×10¹⁰, 3.2×10¹⁰, 3.3×10¹⁰, 3.4×10¹⁰, 3.5×10¹⁰, 3.6×10¹⁰, 3.7×10¹⁰, 3.8×10¹⁰, 3.9×10¹⁰, 4×10¹⁰, 5×10¹⁰, 6×10¹⁰, 7×10¹⁰, 8×10¹⁰, 9×10¹⁰, 1×10¹¹, 1.1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 3.1×10¹¹, 3.2×10¹¹, 3.3×10¹¹, 3.4×10¹¹, 3.5×10¹¹, 3.6×10¹¹, 3.7×10¹¹, 3.8×10¹¹, 3.9×10¹¹, 4×10¹¹ 5×10¹¹, 6×10¹¹, 7×10¹¹, 8×10¹¹, 9×10¹¹, 1×10¹², 1.5×10¹² colony forming units (CFU) of Veillonella parvula (e.g., Veillonella parvula Strain A).

In some embodiments, the pharmaceutical composition comprises at least 1 × 10⁵, 5 × 10⁵, 1 × 10⁶, 2× 10⁶, 3 × 10⁶, 4× 10⁶, 5 × 10⁶, 6 × 10⁶, 7 × 10⁶, 8 × 10⁶, 9 × 10⁶, 1 × 10⁷, 2 × 10⁷, 3 × 10⁷, 4 × 10⁷, 5 × 10⁷, 6 × 10⁷, 7× 10⁷, 8 × 10⁷, 9 × 10⁷, 1 × 10⁸, 2 × 10⁸, 3 × 10⁸, 4 × 10⁸, 5 × 10⁸, 6 × 10⁸, 7 × 10⁸, 8 × 10⁸, 9 × 10⁸ or 1 × 10⁹, 1 × 10¹⁰, 2×10¹⁰, 2.1×10¹⁰, 2.2×10¹⁰, 2.3×10¹⁰, 2.4×10¹⁰, 2.5×10¹⁰, 2.6×10¹⁰, 2.7×10¹⁰, 2.8×10¹⁰, 2.9×10¹⁰, 3×10¹⁰, 3.1×10¹⁰, 3.2×10¹⁰, 3.3×10¹⁰, 3.4×10¹⁰, 3.5×10¹⁰, 3.6×10¹⁰, 3.7×10¹⁰, 3.8×10¹⁰, 3.9×10¹⁰, 4×10¹⁰, 5×10¹⁰, 6×10¹⁰, 7×10¹⁰, 8×10¹⁰, 9×10¹⁰, 1×10¹¹, 1.1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 3.1×10¹¹, 3.2×10¹¹, 3.3×10¹¹, 3.4×10¹¹, 3.5×10¹¹, 3.6×10¹¹, 3.7×10¹¹, 3.8×10¹¹, 3.9×10¹¹, 4×10¹¹ 5×10¹¹, 6×10¹¹, 7×10¹¹, 8×10¹¹, 9×10¹¹, 1×10¹², 1.5×10¹² colony forming units (CFU) of Veillonella parvula (e.g., Veillonella parvula Strain A).

In some embodiments, the pharmaceutical composition comprises at most 1 × 10⁵, 5 × 10⁵, 1 × 10⁶, 2 × 10⁶, 3 × 10⁶, 4×10⁶, 5 × 10⁶, 6 × 10⁶, 7 × 10⁶, 8 × 10⁶, 9 × 10⁶, 1 × 10⁷, 2 × 10⁷, 3 × 10⁷, 4 × 10⁷, 5 × 10⁷, 6 × 10⁷, 7× 10⁷, 8 × 10⁷, 9 × 10⁷, 1 × 10⁸, 2 × 10⁸, 3 × 10⁸, 4 × 10⁸, 5 × 10⁸, 6 × 10⁸, 7 × 10⁸, 8 × 10⁸, 9 × 10⁸ or 1 × 10⁹, 1 × 10¹⁰, 2×10¹⁰, 2.1×10¹⁰, 2.2×10¹⁰, 2.3×10¹⁰, 2.4×10¹⁰, 2.5×10¹⁰, 2.6×10¹⁰, 2.7×10¹⁰, 2.8×10¹⁰, 2.9×10¹⁰, 3×10¹⁰, 3.1×10¹⁰, 3.2×10¹⁰, 3.3×10¹⁰, 3.4×10¹⁰, 3.5×10¹⁰, 3.6×10¹⁰, 3.7×10¹⁰, 3.8×10¹⁰, 3.9×10¹⁰, 4×10¹⁰, 5×10¹⁰, 6×10¹⁰, 7×10¹⁰, 8×10¹⁰, 9×10¹⁰, 1×10¹¹, 1.1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 3.1×10¹¹, 3.2×10¹¹, 3.3×10¹¹, 3.4×10¹¹, 3.5×10¹¹, 3.6×10¹¹, 3.7×10¹¹, 3.8×10¹¹, 3.9×10¹¹, 4×10¹¹ 5×10¹¹, 6×10¹¹, 7×10¹¹, 8×10¹¹, 9×10¹¹, 1×10¹², 1.5×10¹² colony forming units (CFU) of Veillonella parvula (e.g., Veillonella parvula Strain A).

In some embodiments, the pharmaceutical composition comprises about 1 × 10⁵, 5 × 10⁵, 1 × 10⁶, 2× 10⁶, 3 × 10⁶, 4×10⁶, 5 × 10⁶, 6 × 10⁶, 7 × 10⁶, 8 × 10⁶, 9 × 10⁶, 1 × 10⁷, 2 × 10⁷, 3 × 10⁷, 4 × 10⁷, 5 × 10⁷, 6 × 10⁷, 7× 10⁷, 8 × 10⁷, 9 × 10⁷, 1 × 10⁸, 2 × 10⁸, 3 × 10⁸, 4 × 10⁸, 5 × 10⁸, 6 × 10⁸, 7 × 10⁸, 8 × 10⁸, 9 × 10⁸ or 1 × 10⁹, 1 × 10¹⁰, 2×10¹⁰, 2.1×10¹⁰, 2.2×10¹⁰, 2.3×10¹⁰, 2.4×10¹⁰, 2.5×10¹⁰, 2.6×10¹⁰, 2.7×10¹⁰, 2.8×10¹⁰, 2.9×10¹⁰, 3×10¹⁰, 3.1×10¹⁰, 3.2×10¹⁰, 3.3×10¹⁰, 3.4×10¹⁰, 3.5×10¹⁰, 3.6×10¹⁰, 3.7×10¹⁰, 3.8×10¹⁰, 3.9×10¹⁰, 4×10¹⁰, 5×10¹⁰, 6×10¹⁰, 7×10¹⁰, 8×10¹⁰, 9×10¹⁰, 1×10¹¹, 1.1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 3.1×10¹¹, 3.2×10¹¹, 3.3×10¹¹, 3.4×10¹¹, 3.5×10¹¹, 3.6×10¹¹, 3.7×10¹¹, 3.8×10¹¹, 3.9×10¹¹, 4×10¹¹ 5×10¹¹, 6×10¹¹, 7×10¹¹, 8×10¹¹, 9×10¹¹, 1×10¹², 1.5×10¹² total cells (total cell count (TCC)) of Veillonella parvula (e.g., Veillonella parvula Strain A) (e.g., TCC can be determined by Coulter counter).

In some embodiments, the pharmaceutical composition comprises at least 1 × 10⁵, 5 × 10⁵, 1 × 10⁶, 2 × 10⁶, 3 × 10⁶, 4 × 10⁶, 5 × 10⁶, 6 × 10⁶, 7 × 10⁶, 8 × 10⁶, 9 × 10⁶, 1 × 10⁷, 2 × 10⁷, 3 × 10⁷ 4 × 10⁷, 5 × 10⁷, 6× 10⁷, 7× 10⁷, 8 × 10⁷, 9 × 10⁷, 1 × 10⁸, 2 × 10⁸, 3 × 10⁸, 4 × 10⁸, 5 × 10⁸, 6 × 10⁸, 7 × 10⁸, 8 × 10⁸, 9 × 10⁸ or 1 × 10⁹, 1 × 10¹⁰, 2×10¹⁰, 2.1×10¹⁰, 2.2×10¹⁰, 2.3×10¹⁰, 2.4×10¹⁰, 2.5×10¹⁰, 2.6×10¹⁰, 2.7×10¹⁰, 2.8×10¹⁰, 2.9×10¹⁰, 3×10¹⁰, 3.1×10¹⁰, 3.2×10¹⁰, 3.3×10¹⁰, 3.4×10¹⁰, 3.5×10¹⁰, 3.6×10¹⁰, 3.7×10¹⁰, 3.8×10¹⁰, 3.9×10¹⁰, 4×10¹⁰, 5×10¹⁰, 6×10¹⁰, 7×10¹⁰, 8×10¹⁰, 9×10¹⁰, 1×10¹¹, 1.1×10¹¹, 1.2×10¹¹, 1.3×10^{11,} 1.4×10¹¹, 1.5×10¹¹, 1.6×10¹¹, 1.7×10¹¹, 1.8×10¹¹, 1.9×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 3.1×10¹¹, 3.2×10¹¹, 3.3×10¹¹, 3.4×10¹¹, 3.5×10¹¹, 3.6×10¹¹, 3.7×10¹¹, 3.8×10¹¹, 3.9×10¹¹, 4×10¹¹ 5×10¹¹, 6×10¹¹, 7×10¹¹, 8×10¹¹, 9×10¹¹, 1×10¹², 1.5×10¹²total cells (total cell count (TCC)) of Veillonella parvula (e.g., Veillonella parvula Strain A) (e.g., TCC can be determined by Coulter counter).

In some embodiments, the pharmaceutical composition comprises at most 1 × 10⁵, 5 × 10⁵, 1 × 10⁶, 2 × 10⁶, 3 × 10⁶, 4 × 10⁶, 5 × 10⁶, 6 × 10⁶, 7 × 10⁶, 8 × 10⁶, 9 × 10⁶, 1 × 10⁷, 2 × 10⁷, 3 × 10⁷, 4 × 10⁷, 5 × 10⁷, 6 × 10⁷, 7 × 10⁷, 8 × 10⁷, 9 × 10⁷, 1 × 10⁸, 2 × 10⁸, 3 × 10⁸, 4 × 10⁸, 5 × 10⁸, 6 × 10⁸, 7 × 10⁸, 8 × 10⁸, 9 × 10⁸ or 1 × 10⁹, 1 × 10¹⁰, 2×10¹⁰, 2.1×10¹⁰, 2.2×10¹⁰, 2.3×10¹⁰, 2.4×10¹⁰, 2.5×10¹⁰, 2.6×10¹⁰, 2.7×10¹⁰, 2.8×10¹⁰, 2.9×10¹⁰, 3×10¹⁰, 3.1×10¹⁰, 3.2×10¹⁰, 3.3×10¹⁰, 3.4×10¹⁰, 3.5×10¹⁰, 3.6×10¹⁰, 3.7×10¹⁰, 3.8×10¹⁰, 3.9×10¹⁰, 4×10¹⁰, 5×10¹⁰, 6×10¹⁰, 7×10¹⁰, 8×10¹⁰, 9×10¹⁰, 1×10¹¹, 1.1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 3.1×10¹¹, 3.2×10¹¹, 3.3×10¹¹, 3.4×10¹¹, 3.5×10¹¹, 3.6×10¹¹, 3.7×10¹¹, 3.8×10¹¹, 3.9×10¹¹, 4×10¹¹ 5×10¹¹, 6×10¹¹, 7×10¹¹, 8×10¹¹, 9×10¹¹, 1×10¹², 1.5×10¹² total cells (total cell count (TCC)) of Veillonella parvula (e.g., Veillonella parvula Strain A) (e.g., TCC can be determined by Coulter counter).

In some embodiments, the pharmaceutical composition comprises live, killed, attenuated, lyophilized, and/or irradiated (e.g., UV or gamma irradiated) bacteria. Bacteria may be heat-killed by pasteurization, sterilization, high temperature treatment, spray cooking and/or spray drying (heat treatments can be performed at 50° C., 65° C., 85° C. or a variety of other temperatures and/or a varied amount of time). Bacteria may also be killed or inactivated using γ-irradiation (gamma irradiation), exposure to UV light, formalin-inactivation, and/or freezing methods, or a combination thereof. For example, the bacteria may be exposed to 1, 2, 3, 4, 5, 10, 15, 20, 25, 30, 35, 40, or 50 kGy of radiation prior to administration. In some embodiments, bacteria are killed using gamma irradiation. In some embodiments, the bacteria are killed or inactivated using electron irradiation (e.g., beta radiation) or x-ray irradiation.

In some embodiments, the bacteria in the pharmaceutical composition described herein are killed using a method that leaves the disease modulating activity of the bacteria intact and the resulting bacterial components are used in the methods and compositions described herein. In some embodiments, the bacteria in the composition described herein are killed using an antibiotic (e.g., using an antibiotic described herein). In some embodiments, the bacteria in the composition described herein are killed using UV irradiation.

In some embodiments, the bacteria in the composition described herein are killed using heat (temperature) sterilization, filtration, and radiation using methods known to those skilled in the art (Garg M., see the World Wide Web at biologydiscussion.com/microorganisms/sterilizatiion/top-3-physical-methods-used-tokill-microorganisms/55243). The bacteria may be killed via E-beam using methods known to those skilled in the art (SİLİNDİR M. et al, FABAD J. Pharm. Sci., 34, 43-53, 2009). In some embodiments, the bacteria in the composition described herein are killed and/or attenuated by a chemical agent, for example, aldehydes, e.g., formaldehyde, glutaraldehyde, and the like; food preservative agents such as SO₂, sorbic acid, benzoic, acid, nitrate, and nitrite salts; gases such as ethylene oxide; halogens, such as iodine, chlorine, and the like; peroxygens, such as ozone, peroxide, peracetic acid; bisphenols; phenols; phenolics; biguanides, e.g., chlorhexidine; and the like.

Bacteria may be grown to various growth phases and tested for efficacy at different dilutions and at different points during the growth phase. For example, bacteria may be tested for efficacy following administration at stationary phase (including early or late stationary phase), or at various timepoints during exponential phase. In addition to inactivation by various methods, bacteria may be tested for efficacy using different ratios of live versus inactivated cells, or different ratios of cells at various growth phases.

In certain embodiments, provided herein are pharmaceutical compositions comprising mEVs (such as smEVs and/or pmEVs) (e.g., an mEV composition (e.g., an smEV composition or a pmEV composition)) from Veillonella parvula (e.g., Veillonella parvula Strain A). In some embodiments, the mEV composition comprises mEVs (such as smEVs and/or pmEVs) and/or a combination of mEVs (such as smEVs and/or pmEVs) described herein and a pharmaceutically acceptable carrier. In some embodiments, the smEV composition comprises smEVs and/or a combination of smEVs described herein and a pharmaceutically acceptable carrier. In some embodiments, the pmEV composition comprises pmEVs and/or a combination of pmEVs described herein and a pharmaceutically acceptable carrier.

In some embodiments, the pharmaceutical compositions comprise mEVs (such as smEVs and/or pmEVs) substantially or entirely free of whole bacteria (e.g., live bacteria, killed bacteria, attenuated bacteria) from Veillonella parvula (e.g., Veillonella parvula Strain A). In some embodiments, the pharmaceutical compositions comprise whole bacteria (e.g., live bacteria, killed bacteria, attenuated bacteria) from Veillonella parvula (e.g., Veillonella parvula Strain A). In some embodiments, the pharmaceutical compositions comprise both mEVs and whole bacteria (e.g., live bacteria, killed bacteria, attenuated bacteria) from Veillonella parvula (e.g., Veillonella parvula Strain A). In some embodiments, the pharmaceutical composition comprises lyophilized mEVs (such as smEVs and/or pmEVs). In some embodiments, the pharmaceutical composition comprises lyophilized whole bacteria. In some embodiments, the pharmaceutical composition comprises gamma irradiated whole bacteria. In some embodiments, the pharmaceutical composition comprises gamma irradiated mEVs (such as smEVs and/or pmEVs). The mEVs (such as smEVs and/or pmEVs) can be gamma irradiated after the mEVs are isolated (e.g., prepared). In some embodiments, the pharmaceutical compositions comprise mEVs from Veillonella parvula Strain A.

In some embodiments, to quantify the numbers of mEVs (such as smEVs and/or pmEVs) and/or bacteria present in a bacterial sample, electron microscopy (e.g., EM of ultrathin frozen sections) can be used to visualize the mEVs (such as smEVs and/or pmEVs) and/or bacteria and count their relative numbers. Alternatively, nanoparticle tracking analysis (NTA), Coulter counting, or dynamic light scattering (DLS) or a combination of these techniques can be used. NTA and the Coulter counter count particles and show their sizes. DLS gives the size distribution of particles, but not the concentration. Bacteria frequently have diameters of 1-2 um (microns). The full range is 0.2-20 um. Combined results from Coulter counting and NTA can reveal the numbers of bacteria and/or mEVs (such as smEVs and/or pmEVs) in a given sample. Coulter counting reveals the numbers of particles with diameters of 0.7-10 um. For most bacterial and/or mEV (such as smEV and/or pmEV) samples, the Coulter counter alone can reveal the number of bacteria and/or mEVs (such as smEVs and/or pmEVs) in a sample. pmEVs are 20-600 nm in diameter. For NTA, a Nanosight instrument can be obtained from Malvern Pananlytical. For example, the NS300 can visualize and measure particles in suspension in the size range 10-2000 nm. NTA allows for counting of the numbers of particles that are, for example, 50-1000 nm in diameter. DLS reveals the distribution of particles of different diameters within an approximate range of 1 nm - 3 um.

mEVs can be characterized by analytical methods known in the art (e.g., Jeppesen, et al. Cell 177:428 (2019)).

In some embodiments, the mEVs may be quantified based on particle count. For example, total protein content of an mEV preparation can be measured using NTA.

In some embodiments, the mEVs may be quantified based on the amount of protein, lipid, or carbohydrate. For example, a dose of mEV can be determined by particle count of an mEV preparation can be measured using the Bradford assay or the BCA assay.

In some embodiments, the mEVs are isolated away from one or more other bacterial components of the source bacteria. In some embodiments, the pharmaceutical composition further comprises other bacterial components.

In certain embodiments, the mEV preparation obtained from the source bacteria may be fractionated into subpopulations based on the physical properties (e.g., sized, density, protein content, binding affinity) of the subpopulations. One or more of the mEV subpopulations can then be incorporated into the pharmaceutical compositions of the invention.

In certain aspects, provided herein are pharmaceutical compositions comprising mEVs (such as smEVs and/or pmEVs) useful for the treatment and/or prevention of disease (e.g., a neuroinflammatory disease, a neurodegenerative disease, a neuromuscular disease, and/or a psychiatric disorder), as well as methods of making and/or identifying such mEVs, and methods of using such pharmaceutical compositions (e.g., for the treatment and/or prevention of a disease or a health disorder (e.g., a neuroinflammatory disease, a neurodegenerative disease, a neuromuscular disease, and/or a psychiatric disorder), either alone or in combination with other therapeutics). In some embodiments, the pharmaceutical compositions comprise both mEVs (such as smEVs and/or pmEVs), and whole bacteria (e.g., live bacteria, killed bacteria, attenuated bacteria). In some embodiments, the pharmaceutical compositions comprise mEVs (such as smEVs and/or pmEVs) in the absence of bacteria. In some embodiments, the pharmaceutical compositions comprise mEVs (such as smEVs and/or pmEVs) and/or bacteria from Veillonella parvula Strain A.

In certain aspects, provided herein are pharmaceutical compositions comprising Veillonella parvula bacteria provided and Veillonella parvula mEVs described herein. In some embodiments, the bacteria is Veillonella parvula Strain A.

In some embodiments, the pharmaceutical composition comprises at least 1 Veillonella parvula (e.g., Veillonella parvula Strain A) bacterium for every 1, 1.1, 1.2, 1.3, 1.4, 1.5, 1.6, 1.7, 1.8. 1.9, 2, 2.1, 2.2, 2.3, 2.4, 2.5, 2.6, 2.7, 2.8. 2.9, 3, 3.1, 3.2, 3.3, 3.4, 3.5, 3.6, 3.7, 3.8. 3.9, 4, 4.1, 4.2, 4.3, 4.4, 4.5, 4.6, 4.7, 4.8. 4.9, 5, 5.1, 5.2, 5.3, 5.4, 5.5, 5.6, 5.7, 5.8. 5.9, 6, 6.1, 6.2, 6.3, 6.4, 6.5, 6.6, 6.7, 6.8. 6.9, 7, 7.1, 7.2, 7.3, 7.4, 7.5, 7.6, 7.7, 7.8. 7.9, 8, 8.1, 8.2, 8.3, 8.4, 8.5, 8.6, 8.7, 8.8. 8.9, 9, 9.1, 9.2, 9.3, 9.4, 9.5, 9.6, 9.7, 9.8. 9.9, 10, 11, 12, 13, 14, 15, 16, 17, 18. 19, 20, 21, 22, 23, 24, 25, 26, 27, 28. 29, 30, 31, 32, 33, 34, 35, 36, 37, 38. 39, 40, 41, 42, 43, 44, 45, 46, 47, 48. 49, 50, 51, 52, 53, 54, 55, 56, 57, 58. 59, 60, 61, 62, 63, 64, 65, 66, 67, 68. 69, 70, 71, 72, 73, 74, 75, 76, 77, 78. 79, 80, 81, 82, 83, 84, 85, 86, 87, 88. 89, 90, 91, 92, 93, 94, 95, 96, 97, 98. 99, 100, 150, 200, 250, 300, 350, 400, 450, 500, 550, 600, 650, 700, 750, 800, 850, 900, 950, 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, and/or 1×10¹² Veillonella parvula (e.g., Veillonella parvula Strain A) mEV particles.

In some embodiments, the pharmaceutical composition comprises about 1 Veillonella parvula (e.g., Veillonella parvula Strain A) bacterium for every 1, 1.1, 1.2, 1.3, 1.4, 1.5, 1.6, 1.7, 1.8. 1.9, 2, 2.1, 2.2, 2.3, 2.4, 2.5, 2.6, 2.7, 2.8. 2.9, 3, 3.1, 3.2, 3.3, 3.4, 3.5, 3.6, 3.7, 3.8. 3.9, 4, 4.1, 4.2, 4.3, 4.4, 4.5, 4.6, 4.7, 4.8. 4.9, 5, 5.1, 5.2, 5.3, 5.4, 5.5, 5.6, 5.7, 5.8. 5.9, 6, 6.1, 6.2, 6.3, 6.4, 6.5, 6.6, 6.7, 6.8. 6.9, 7, 7.1, 7.2, 7.3, 7.4, 7.5, 7.6, 7.7, 7.8. 7.9, 8, 8.1, 8.2, 8.3, 8.4, 8.5, 8.6, 8.7, 8.8. 8.9, 9, 9.1, 9.2, 9.3, 9.4, 9.5, 9.6, 9.7, 9.8. 9.9, 10, 11, 12, 13, 14, 15, 16, 17, 18. 19, 20, 21, 22, 23, 24, 25, 26, 27, 28. 29, 30, 31, 32, 33, 34, 35, 36, 37, 38. 39, 40, 41, 42, 43, 44, 45, 46, 47, 48. 49, 50, 51, 52, 53, 54, 55, 56, 57, 58. 59, 60, 61, 62, 63, 64, 65, 66, 67, 68. 69, 70, 71, 72, 73, 74, 75, 76, 77, 78. 79, 80, 81, 82, 83, 84, 85, 86, 87, 88. 89, 90, 91, 92, 93, 94, 95, 96, 97, 98. 99, 100, 150, 200, 250, 300, 350, 400, 450, 500, 550, 600, 650, 700, 750, 800, 850, 900, 950, 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, and/or 1×10¹² Veillonella parvula (e.g., Veillonella parvula Strain A) mEV particles.

In some embodiments, the pharmaceutical composition comprises no more than 1 Veillonella parvula (e.g., Veillonella parvula Strain A) bacterium for every 1, 1.1, 1.2, 1.3, 1.4, 1.5, 1.6, 1.7, 1.8. 1.9, 2, 2.1, 2.2, 2.3, 2.4, 2.5, 2.6, 2.7, 2.8. 2.9, 3, 3.1, 3.2, 3.3, 3.4, 3.5, 3.6, 3.7, 3.8. 3.9, 4, 4.1, 4.2, 4.3, 4.4, 4.5, 4.6, 4.7, 4.8. 4.9, 5, 5.1, 5.2, 5.3, 5.4, 5.5, 5.6, 5.7, 5.8. 5.9, 6, 6.1, 6.2, 6.3, 6.4, 6.5, 6.6, 6.7, 6.8. 6.9, 7, 7.1, 7.2, 7.3, 7.4, 7.5, 7.6, 7.7, 7.8. 7.9, 8, 8.1, 8.2, 8.3, 8.4, 8.5, 8.6, 8.7, 8.8. 8.9, 9, 9.1, 9.2, 9.3, 9.4, 9.5, 9.6, 9.7, 9.8. 9.9, 10, 11, 12, 13, 14, 15, 16, 17, 18. 19, 20, 21, 22, 23, 24, 25, 26, 27, 28. 29, 30, 31, 32, 33, 34, 35, 36, 37, 38. 39, 40, 41, 42, 43, 44, 45, 46, 47, 48. 49, 50, 51, 52, 53, 54, 55, 56, 57, 58. 59, 60, 61, 62, 63, 64, 65, 66, 67, 68. 69, 70, 71, 72, 73, 74, 75, 76, 77, 78. 79, 80, 81, 82, 83, 84, 85, 86, 87, 88. 89, 90, 91, 92, 93, 94, 95, 96, 97, 98. 99, 100, 150, 200, 250, 300, 350, 400, 450, 500, 550, 600, 650, 700, 750, 800, 850, 900, 950, 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵ 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, and/or 1×10¹² Veillonella parvula (e.g., Veillonella parvula Strain A) mEV particles.

In some embodiments, the pharmaceutical composition comprises at least 1 Veillonella parvula (e.g., Veillonella parvula Strain A) mEV particle for every 1, 1.1, 1.2, 1.3, 1.4, 1.5, 1.6, 1.7, 1.8. 1.9, 2, 2.1, 2.2, 2.3, 2.4, 2.5, 2.6, 2.7, 2.8. 2.9, 3, 3.1, 3.2, 3.3, 3.4, 3.5, 3.6, 3.7, 3.8. 3.9, 4, 4.1, 4.2, 4.3, 4.4, 4.5, 4.6, 4.7, 4.8. 4.9, 5, 5.1, 5.2, 5.3, 5.4, 5.5, 5.6, 5.7, 5.8. 5.9, 6, 6.1, 6.2, 6.3, 6.4, 6.5, 6.6, 6.7, 6.8. 6.9, 7, 7.1, 7.2, 7.3, 7.4, 7.5, 7.6, 7.7, 7.8. 7.9, 8, 8.1, 8.2, 8.3, 8.4, 8.5, 8.6, 8.7, 8.8. 8.9, 9, 9.1, 9.2, 9.3, 9.4, 9.5, 9.6, 9.7, 9.8. 9.9, 10, 11, 12, 13, 14, 15, 16, 17, 18. 19, 20, 21, 22, 23, 24, 25, 26, 27, 28. 29, 30, 31, 32, 33, 34, 35, 36, 37, 38. 39, 40, 41, 42, 43, 44, 45, 46, 47, 48. 49, 50, 51, 52, 53, 54, 55, 56, 57, 58. 59, 60, 61, 62, 63, 64, 65, 66, 67, 68. 69, 70, 71, 72, 73, 74, 75, 76, 77, 78. 79, 80, 81, 82, 83, 84, 85, 86, 87, 88. 89, 90, 91, 92, 93, 94, 95, 96, 97, 98. 99, 100, 150, 200, 250, 300, 350, 400, 450, 500, 550, 600, 650, 700, 750, 800, 850, 900, 950, 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵ 3×10⁵, 4×10⁵ 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, and/or 1×10¹² Veillonella parvula (e.g., Veillonella parvula Strain A) bacteria.

In some embodiments, the pharmaceutical composition comprises about 1 Veillonella parvula (e.g., Veillonella parvula Strain A) mEV particle for every 1, 1.1, 1.2, 1.3, 1.4, 1.5, 1.6, 1.7, 1.8. 1.9, 2, 2.1, 2.2, 2.3, 2.4, 2.5, 2.6, 2.7, 2.8. 2.9, 3, 3.1, 3.2, 3.3, 3.4, 3.5, 3.6, 3.7, 3.8. 3.9, 4, 4.1, 4.2, 4.3, 4.4, 4.5, 4.6, 4.7, 4.8. 4.9, 5, 5.1, 5.2, 5.3, 5.4, 5.5, 5.6, 5.7, 5.8. 5.9, 6, 6.1, 6.2, 6.3, 6.4, 6.5, 6.6, 6.7, 6.8. 6.9, 7, 7.1, 7.2, 7.3, 7.4, 7.5, 7.6, 7.7, 7.8. 7.9, 8, 8.1, 8.2, 8.3, 8.4, 8.5, 8.6, 8.7, 8.8. 8.9, 9, 9.1, 9.2, 9.3, 9.4, 9.5, 9.6, 9.7, 9.8. 9.9, 10, 11, 12, 13, 14, 15, 16, 17, 18. 19, 20, 21, 22, 23, 24, 25, 26, 27, 28. 29, 30, 31, 32, 33, 34, 35, 36, 37, 38. 39, 40, 41, 42, 43, 44, 45, 46, 47, 48. 49, 50, 51, 52, 53, 54, 55, 56, 57, 58. 59, 60, 61, 62, 63, 64, 65, 66, 67, 68. 69, 70, 71, 72, 73, 74, 75, 76, 77, 78. 79, 80, 81, 82, 83, 84, 85, 86, 87, 88. 89, 90, 91, 92, 93, 94, 95, 96, 97, 98. 99, 100, 150, 200, 250, 300, 350, 400, 450, 500, 550, 600, 650, 700, 750, 800, 850, 900, 950, 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵ 3×10⁵, 4×10⁵ 5×10⁵, 6×10⁵, 7×10⁵, 8×10⁵, 9×10⁵, 1×10⁶, 2×10⁶, 3×10⁶ 4×10⁶ 5×10⁶, 6×10⁶ 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10^{9,} 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, and/or 1×10¹² Veillonella parvula (e.g., Veillonella parvula Strain A) bacteria.

In some embodiments, the pharmaceutical composition comprises no more than 1 Veillonella parvula (e.g., Veillonella parvula Strain A) mEV particle for every 1, 1.1, 1.2, 1.3, 1.4, 1.5, 1.6, 1.7, 1.8. 1.9, 2, 2.1, 2.2, 2.3, 2.4, 2.5, 2.6, 2.7, 2.8. 2.9, 3, 3.1, 3.2, 3.3, 3.4, 3.5, 3.6, 3.7, 3.8. 3.9, 4, 4.1, 4.2, 4.3, 4.4, 4.5, 4.6, 4.7, 4.8. 4.9, 5, 5.1, 5.2, 5.3, 5.4, 5.5, 5.6, 5.7, 5.8. 5.9, 6, 6.1, 6.2, 6.3, 6.4, 6.5, 6.6, 6.7, 6.8. 6.9, 7, 7.1, 7.2, 7.3, 7.4, 7.5, 7.6, 7.7, 7.8. 7.9, 8, 8.1, 8.2, 8.3, 8.4, 8.5, 8.6, 8.7, 8.8. 8.9, 9, 9.1, 9.2, 9.3, 9.4, 9.5, 9.6, 9.7, 9.8. 9.9, 10, 11, 12, 13, 14, 15, 16, 17, 18. 19, 20, 21, 22, 23, 24, 25, 26, 27, 28. 29, 30, 31, 32, 33, 34, 35, 36, 37, 38. 39, 40, 41, 42, 43, 44, 45, 46, 47, 48. 49, 50, 51, 52, 53, 54, 55, 56, 57, 58. 59, 60, 61, 62, 63, 64, 65, 66, 67, 68. 69, 70, 71, 72, 73, 74, 75, 76, 77, 78. 79, 80, 81, 82, 83, 84, 85, 86, 87, 88. 89, 90, 91, 92, 93, 94, 95, 96, 97, 98. 99, 100, 150, 200, 250, 300, 350, 400, 450, 500, 550, 600, 650, 700, 750, 800, 850, 900, 950, 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10³, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, and/or 1×10¹² Veillonella parvula (e.g., Veillonella parvula Strain A) bacteria.

In some embodiments, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the total particles in the pharmaceutical composition are Veillonella parvula (e.g., Veillonella parvula Strain A) mEVs.

In some embodiments, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the total particles in the pharmaceutical composition are Veillonella parvula (e.g., Veillonella parvula Strain A) bacteria.

In some embodiments, no more than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of total the particles in the pharmaceutical composition are Veillonella parvula (e.g., Veillonella parvula Strain A) mEVs.

In some embodiments, no more than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the total particles in the pharmaceutical composition are Veillonella parvula (e.g., Veillonella parvula Strain A) bacteria.

In some embodiments, about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the total particles in the pharmaceutical composition are Veillonella parvula (e.g., Veillonella parvula Strain A) mEVs.

In some embodiments, about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the total particles in the pharmaceutical composition are Veillonella parvula (e.g., Veillonella parvula Strain A) bacteria.

In some embodiments, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the total protein in the pharmaceutical composition is Veillonella parvula (e.g., Veillonella parvula Strain A) mEV protein.

In some embodiments, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the total protein in the pharmaceutical composition is Veillonella parvula (e.g., Veillonella parvula Strain A) bacteria protein.

In some embodiments, no more than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the total protein in the pharmaceutical composition is Veillonella parvula (e.g., Veillonella parvula Strain A) mEV protein.

In some embodiments, no more than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the total protein in the pharmaceutical composition is Veillonella parvula (e.g., Veillonella parvula Strain A) bacteria protein.

In some embodiments, about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the total protein in the pharmaceutical composition is Veillonella parvula (e.g., Veillonella parvula Strain A) mEV protein.

In some embodiments, about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the total protein in the pharmaceutical composition is Veillonella parvula (e.g., Veillonella parvula Strain A) bacteria protein.

In some embodiments, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the total lipids in the pharmaceutical composition are Veillonella parvula (e.g., Veillonella parvula Strain A) mEV lipids.

In some embodiments, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the total lipids in the pharmaceutical composition are Veillonella parvula (e.g., Veillonella parvula Strain A) bacteria lipids.

In some embodiments, no more than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the total lipids in the pharmaceutical composition are Veillonella parvula (e.g., Veillonella parvula Strain A) mEV lipids.

In some embodiments, no more than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the total lipids in the pharmaceutical composition are Veillonella parvula (e.g., Veillonella parvula Strain A) bacteria lipids.

In some embodiments, about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the total lipids in the pharmaceutical composition are Veillonella parvula (e.g., Veillonella parvula Strain A) mEV lipids.

In some embodiments, about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the total lipids in the pharmaceutical composition are Veillonella parvula (e.g., Veillonella parvula Strain A) bacteria lipids.

In certain aspects, provided are pharmaceutical compositions for administration to a subject (e.g., human subject). In some embodiments, the pharmaceutical compositions are combined with additional active and/or inactive materials in order to produce a final product, which may be in single dosage unit or in a multi-dose format. In some embodiments, the pharmaceutical composition is combined with an adjuvant such as an immuno-adjuvant (e.g., a STING agonist, a TLR agonist, or a NOD agonist).

In some embodiments, the pharmaceutical composition comprises at least one carbohydrate.

In some embodiments, the pharmaceutical composition comprises at least one lipid. In some embodiments the lipid comprises at least one fatty acid selected from lauric acid (12:0), myristic acid (14:0), palmitic acid (16:0), palmitoleic acid (16:1), margaric acid (17:0), heptadecenoic acid (17:1), stearic acid (18:0), oleic acid (18:1), linoleic acid (18:2), linolenic acid (18:3), octadecatetraenoic acid (18:4), arachidic acid (20:0), eicosenoic acid (20:1), eicosadienoic acid (20:2), eicosatetraenoic acid (20:4), eicosapentaenoic acid (20:5) (EPA), docosanoic acid (22:0), docosenoic acid (22:1), docosapentaenoic acid (22:5), docosahexaenoic acid (22:6) (DHA), and tetracosanoic acid (24:0).

In some embodiments, the pharmaceutical composition comprises at least one supplemental mineral or mineral source. Examples of minerals include, without limitation: chloride, sodium, calcium, iron, chromium, copper, iodine, zinc, magnesium, manganese, molybdenum, phosphorus, potassium, and selenium. Suitable forms of any of the foregoing minerals include soluble mineral salts, slightly soluble mineral salts, insoluble mineral salts, chelated minerals, mineral complexes, non-reactive minerals such as carbonyl minerals, and reduced minerals, and combinations thereof.

In some embodiments, the pharmaceutical composition comprises at least one supplemental vitamin. The at least one vitamin can be fat-soluble or water soluble vitamins. Suitable vitamins include but are not limited to vitamin C, vitamin A, vitamin E, vitamin B12, vitamin K, riboflavin, niacin, vitamin D, vitamin B6, folic acid, pyridoxine, thiamine, pantothenic acid, and biotin. Suitable forms of any of the foregoing are salts of the vitamin, derivatives of the vitamin, compounds having the same or similar activity of the vitamin, and metabolites of the vitamin.

In some embodiments, the pharmaceutical composition comprises an excipient. Non-limiting examples of suitable excipients include a buffering agent, a preservative, a stabilizer, a binder, a compaction agent, a lubricant, a dispersion enhancer, a disintegration agent, a flavoring agent, a sweetener, and a coloring agent.

In some embodiments, the excipient is a buffering agent. Non-limiting examples of suitable buffering agents include sodium citrate, magnesium carbonate, magnesium bicarbonate, calcium carbonate, and calcium bicarbonate.

In some embodiments, the excipient comprises a preservative. Non-limiting examples of suitable preservatives include antioxidants, such as alpha-tocopherol and ascorbate, and antimicrobials, such as parabens, chlorobutanol, and phenol.

In some embodiments, the pharmaceutical composition comprises a binder as an excipient. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, and combinations thereof.

In some embodiments, the pharmaceutical composition comprises a lubricant as an excipient. Non-limiting examples of suitable lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

In some embodiments, the pharmaceutical composition comprises a dispersion enhancer as an excipient. Non-limiting examples of suitable dispersants include starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

In some embodiments, the pharmaceutical composition comprises a disintegrant as an excipient. In some embodiments the disintegrant is a non-effervescent disintegrant. Non-limiting examples of suitable non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pectin, and tragacanth. In some embodiments the disintegrant is an effervescent disintegrant. Non-limiting examples of suitable effervescent disintegrants include sodium bicarbonate in combination with citric acid, and sodium bicarbonate in combination with tartaric acid.

In some embodiments, the pharmaceutical composition is a food product (e.g., a food or beverage) such as a health food or beverage, a food or beverage for infants, a food or beverage for pregnant women, athletes, senior citizens or other specified group, a functional food, a beverage, a food or beverage for specified health use, a dietary supplement, a food or beverage for patients, or an animal feed. Specific examples of the foods and beverages include various beverages such as juices, refreshing beverages, tea beverages, drink preparations, jelly beverages, and functional beverages; alcoholic beverages such as beers; carbohydrate-containing foods such as rice food products, noodles, breads, and pastas; paste products such as fish hams, sausages, paste products of seafood; retort pouch products such as curries, food dressed with a thick starchy sauces, and Chinese soups; soups; dairy products such as milk, dairy beverages, ice creams, cheeses, and yogurts; fermented products such as fermented soybean pastes, yogurts, fermented beverages, and pickles; bean products; various confectionery products, including biscuits, cookies, and the like, candies, chewing gums, gummies, cold desserts including jellies, cream caramels, and frozen desserts; instant foods such as instant soups and instant soy-bean soups; microwavable foods; and the like. Further, the examples also include health foods and beverages prepared in the forms of powders, granules, tablets, capsules, liquids, pastes, and jellies.

In some embodiments, the pharmaceutical composition is a food product for animals, including humans. The animals, other than humans, are not particularly limited, and the composition can be used for various livestock, poultry, pets, experimental animals, and the like. Specific examples of the animals include pigs, cattle, horses, sheep, goats, chickens, wild ducks, ostriches, domestic ducks, dogs, cats, rabbits, hamsters, mice, rats, monkeys, and the like, but the animals are not limited thereto.

### Dose Forms

A pharmaceutical composition comprising mEVs (such as smEVs and/or pmEVs), bacteria, or any combination thereof, from Veillonella parvula (e.g., Veillonella parvula Strain A) can be formulated as a solid dose form, e.g., for oral administration. The solid dose form can comprise one or more excipients, e.g., pharmaceutically acceptable excipients. The mEVs (such as smEVs and/or pmEVs), bacteria, or any combination thereof, in the solid dose form can be isolated mEVs (such as smEVs and/or pmEVs), bacteria, or any combination thereof. Optionally, the mEVs (such as smEVs and/or pmEVs), bacteria, or any combination thereof, in the solid dose form can be lyophilized. Optionally, the mEVs (such as smEVs and/or pmEVs), bacteria, or any combination thereof, in the solid dose form are gamma irradiated. The solid dose form can comprise a tablet, a minitablet, a capsule, a pill, or a powder; or a combination of these forms (e.g., minitablets comprised in a capsule).

The solid dose form can comprise a tablet (e.g., > 4 mm).

The solid dose form can comprise a mini tablet (e.g., 1-4 mm sized minitablet, e.g., a 2 mm minitablet or a 3 mm minitablet).

The solid dose form can comprise a capsule, e.g., a size 00, size 0, size 1, size 2, size 3, size 4, or size 5 capsule; e.g., a size 0 capsule.

The solid dose form can comprise a coating. The solid dose form can comprise a single layer coating, e.g., enteric coating, e.g., a Eudragit-based coating, e.g., EUDRAGIT L30 D-55, triethylcitrate, and talc. The solid dose form can comprise two layers of coating. For example, an inner coating can comprise, e.g., EUDRAGIT L30 D-55, triethylcitrate, talc, citric acid anhydrous, and sodium hydroxide, and an outer coating can comprise, e.g., EUDRAGIT L30 D-55, triethylcitrate, and talc. EUDRAGIT is the brand name for a diverse range of polymethacrylate-based copolymers. It includes anionic, cationic, and neutral copolymers based on methacrylic acid and methacrylic/acrylic esters or their derivatives. Eudragits are amorphous polymers having glass transition temperatures between 9 to > 150° C. Eudragits are non-biodegradable, nonabsorbable, and nontoxic. Anionic Eudragit L dissolves at pH > 6 and is used for enteric coating, while Eudragit S, soluble at pH > 7 is used for colon targeting. Eudragit RL and RS, having quaternary ammonium groups, are water insoluble, but swellable/permeable polymers which are suitable for the sustained release film coating applications. Cationic Eudragit E, insoluble at pH ≥ 5, can prevent drug release in saliva.

The solid dose form (e.g., a capsule) can comprise a single layer coating, e.g., a non-enteric coating such as HPMC (hydroxyl propyl methyl cellulose) or gelatin.

A pharmaceutical composition comprising mEVs (such as smEVs and/or pmEVs), bacteria, or any combination thereof, from Veillonella parvula (e.g., Veillonella parvula Strain A) can be formulated as a suspension, e.g., for oral administration or for injection. Administration by injection includes intravenous (IV), intramuscular (IM), and subcutaneous (SC) administration. For a suspension, mEVs (such as smEVs and/or pmEVs), bacteria, or any combination thereof, can be in a buffer, e.g., a pharmaceutically acceptable buffer, e.g., saline or PBS. The suspension can comprise one or more excipients, e.g., pharmaceutically acceptable excipients. The suspension can comprise, e.g., sucrose or glucose. The mEVs (such as smEVs and/or pmEVs), bacteria, or any combination thereof, in the suspension can be isolated mEVs (such as smEVs and/or pmEVs), bacteria, or any combination thereof. Optionally, the mEVs (such as smEVs and/or pmEVs), bacteria, or any combination thereof, in the suspension can be lyophilized. Optionally, the mEVs (such as smEVs and/or pmEVs), bacteria, or any combination thereof, in the suspension can be gamma irradiated.

### Dosage

For oral administration to a human subject, the dose of bacteria from Veillonella parvula (e.g., Veillonella parvula Strain A) can be, e.g., about 1×10⁵- about 2×10¹⁶ total cells (total cell count (TCC)). The dose can be, e.g., about 1 × 10⁵, 5 × 10⁵, 1 × 10⁶, 2 x 10⁶, 3 × 10⁶, 4 × 10⁶, 5 × 10⁶, 6 × 10⁶, 7 × 10⁶, 8 × 10⁶, 9 × 10⁶, 1 × 10⁷, 2 × 10⁷, 3 × 10⁷, 4 × 10⁷, 5 × 10⁷, 6 × 10⁷, 7 × 10⁷, 8 × 10⁷, 9 × 10⁷, 1 × 10⁸, 2 × 10⁸, 3 × 10⁸, 4 × 10⁸, 5 × 10⁸, 6 × 10⁸, 7 × 10⁸, 8 × 10⁸, 9 × 10⁸ or 1 × 10⁹, 1 × 10¹⁰, 2×10¹⁰, 2.1×10¹⁰, 2.2×10¹⁰, 2.3×10¹⁰, 2.4×10¹⁰, 2.5×10¹⁰, 2.6×10¹⁰, 2.7×10¹⁰, 2.8×10¹⁰, 2.9×10¹⁰, 3×10¹⁰, 3.1×10¹⁰ 3.2×10¹⁰, 3.3×10¹⁰, 3.4×10¹⁰, 3.5×10¹⁰, 3.6×10¹⁰, 3.7×10¹⁰, 3.8×10¹⁰, 3.9×10¹⁰, 4×10¹⁰, 5×10¹⁰, 6×10¹⁰, 7×10¹⁰, 8×10¹⁰, 9×10¹⁰, 1×10¹¹, 1.1×10¹¹, 1.2×10¹¹, 1.3×10¹¹, 1.4×10¹¹, 1.5×10¹¹, 1.6×10^{11,} 1.7×10¹¹, 1.8×10¹¹, 1.9×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 3.1×10¹¹, 3.2×10¹¹, 3.3×10¹¹, 3.4×10¹¹, 3.5×10¹¹, 3.6×10¹¹, 3.7×10¹¹, 3.8×10¹¹, 3.9×10¹¹, 4×10¹¹ 5×10¹¹, 6×10¹¹, 7×10¹¹, 8×10¹¹, 9×10¹¹, 1×10¹², 1.5×10¹² total cells (total cell count (TCC)) of Veillonella parvula (e.g., Veillonella parvula Strain A) (e.g., TCC can be determined by Coulter counter).

For oral administration to a human subject, the dose of mEVs (such as smEVs and/or pmEVs), bacteria, or any combination thereof, from Veillonella parvula (e.g., Veillonella parvula Strain A) can be, e.g., about 2×10⁶- about 2×10¹⁶ particles. The dose can be, e.g., about 1×10⁷- about 1×10¹⁵, about 1×10⁸- about 1×10¹⁴, about 1×10⁹- about 1×10¹³, about 1×10¹⁰- about 1×10¹⁴, or about 1×10⁸- about 1×10¹² particles. The dose can be, e.g., about 2×10⁶, about 2×10⁷, about 2×10⁸, about 2×10⁹, about 1×10¹⁰, about 2×10¹⁰, about 2×10¹¹, about 2×10¹², about 2×10¹³, about 2×10¹⁴, or about 1×10¹⁵ particles. The dose can be, e.g., about 2×10¹⁴ particles. The dose can be, e.g., about 2×10¹² particles. The dose can be, e.g., about 2×10¹⁰ particles. The dose can be, e.g., about 1×10¹⁰ particles. Particle count can be determined, e.g., by NTA.

For oral administration to a human subject, the dose of mEVs (such as smEVs and/or pmEVs), bacteria, or any combination thereof, can be, e.g., based on total protein. The dose can be, e.g., about 5 mg to about 900 mg total protein. The dose can be, e.g., about 20 mg to about 800 mg, about 50 mg to about 700 mg, about 75 mg to about 600 mg, about 100 mg to about 500 mg, about 250 mg to about 750 mg, or about 200 mg to about 500 mg total protein. The dose can be, e.g., about 10 mg, about 25 mg, about 50 mg, about 75 mg, about 100 mg, about 150 mg, about 200 mg, about 250 mg, about 300 mg, about 400 mg, about 500 mg, about 600 mg, or about 750 mg total protein. Total protein can be determined, e.g., by Bradford assay or BCA.

For administration by injection (e.g., intravenous administration) to a human subject, the dose of mEVs (such as smEVs and/or pmEVs), bacteria, or any combination thereof, can be, e.g., about 1×10⁶- about 1×10¹⁶ particles. The dose can be, e.g., about 1×10⁷- about 1×10¹⁵, about 1×10⁸- about 1×10¹⁴, about 1×10⁹- about 1×10¹³, about 1×10¹⁰-about 1×10¹⁴, or about 1×10⁸- about 1×10¹² particles. The dose can be, e.g., about 2×10⁶, about 2×10⁷, about 2×10⁸, about 2×10⁹, about 1×10¹⁰, about 2×10¹⁰, about 2×10¹¹, about 2×10¹², about 2×10¹³, about 2×10¹⁴, or about 1×10¹⁵ particles. The dose can be, e.g., about 1×10¹⁵ particles. The dose can be, e.g., about 2×10¹⁴ particles. The dose can be, e.g., about 2×10¹³ particles. Particle count can be determined, e.g., by NTA.

For administration by injection (e.g., intravenous administration), the dose of mEVs (such as smEVs and/or pmEVs), bacteria, or any combination thereof, can be, e.g., about 5 mg to about 900 mg total protein. The dose can be, e.g., about 20 mg to about 800 mg, about 50 mg to about 700 mg, about 75 mg to about 600 mg, about 100 mg to about 500 mg, about 250 mg to about 750 mg, or about 200 mg to about 500 mg total protein. The dose can be, e.g., about 10 mg, about 25 mg, about 50 mg, about 75 mg, about 100 mg, about 150 mg, about 200 mg, about 250 mg, about 300 mg, about 400 mg, about 500 mg, about 600 mg, or about 750 mg total protein. The dose can be, e.g., about 700 mg total protein. The dose can be, e.g., about 350 mg total protein. The dose can be, e.g., about 175 mg total protein. Total protein can be determined, e.g., by Bradford assay or BCA.

### Gamma-Irradiation

Powders (e.g., of mEVs (such as smEVs and/or pmEVs), bacteria, or any combination thereof) can be gamma-irradiated at 17.5 kGy radiation unit at ambient temperature.

Frozen biomasses (e.g., of mEVs (such as smEVs and/or pmEVs), bacteria, or any combination thereof) can be gamma-irradiated at 25 kGy radiation unit in the presence of dry ice.

### Additional Therapeutic Agents

In certain aspects, the methods provided herein include the administration to a subject of a pharmaceutical composition described herein either alone or in combination with an additional therapeutic agent. In some embodiments, the additional therapeutic agent is an immunotherapy agent. In some embodiments, the additional therapeutic agent is a treatment for a neuroinflammatory disease, a neurodegenerative disease, a neuromuscular disease, and/or a psychiatric disorder.

In some embodiments, the pharmaceutical composition comprising mEVs (such as smEVs and/or pmEVs), bacteria, or any combination thereof, from Veillonella parvula (e.g., Veillonella parvula Strain A) is administered to the subject before the additional therapeutic agent is administered (e.g., at least 1, 2, 3, 4, 5, 6, 7, 8, 9, 10, 11, 12, 13, 14, 15, 16, 17, 18, 19, 20, 21, 22, 23 or 24 hours before or at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days before). In some embodiments, the pharmaceutical composition comprising mEVs (such as smEVs and/or pmEVs), bacteria, or any combination thereof, is administered to the subject after the additional therapeutic agent is administered (e.g., at least 1, 2, 3, 4, 5, 6, 7, 8, 9, 10, 11, 12, 13, 14, 15, 16, 17, 18, 19, 20, 21, 22, 23 or 24 hours after or at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days after). In some embodiments, the pharmaceutical composition comprising mEVs (such as smEVs and/or pmEVs), bacteria, or any combination thereof, and the additional therapeutic agent are administered to the subject simultaneously or nearly simultaneously (e.g., administrations occur within an hour of each other).

In some embodiments, an antibiotic is administered to the subject before the pharmaceutical composition comprising mEVs (such as smEVs and/or pmEVs), bacteria, or any combination thereof, is administered to the subject (e.g., at least 1, 2, 3, 4, 5, 6, 7, 8, 9, 10, 11, 12, 13, 14, 15, 16, 17, 18, 19, 20, 21, 22, 23 or 24 hours before or at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days before). In some embodiments, an antibiotic is administered to the subject after pharmaceutical composition comprising mEVs (such as smEVs and/or pmEVs), bacteria, or any combination thereof, is administered to the subject (e.g., at least 1, 2, 3, 4, 5, 6, 7, 8, 9, 10, 11, 12, 13, 14, 15, 16, 17, 18, 19, 20, 21, 22, 23 or 24 hours before or at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days after). In some embodiments, the pharmaceutical composition comprising mEVs (such as smEVs and/or pmEVs), bacteria, or any combination thereof, and the antibiotic are administered to the subject simultaneously or nearly simultaneously (e.g., administrations occur within an hour of each other).

In some embodiments, the additional therapeutic agent is an a treatment for a neuroinflammatory disease, a neurodegenerative disease, a neuromuscular disease, and/or a psychiatric disorder. Non-limiting examples include: a S1P receptor inhibitor (Gilenya), a Nrf2 activator (Tecfidera), or an IV/SubCu-infused biologic (such as Ocrevus, Tysabri, Copaxane, or Avonex).

In some embodiments, non-limiting examples of the additional therapeutic agent that is effective in treating neuroinflammatory disease, a neurodegenerative disease, a neuromuscular disease, and/or a psychiatric disorder include interferon-β, glatiramer acetate, mitoxantrone, glucocorticoids, palmitoylethanolamide (PEA), melatonin, minocycline, statins, aspirin, celecoxib, risperidone, olanzapine, paracetamol, COX-2 inhibitors, sodium valproate, escitalopram, nortriptyline, sodium naproxen, fluvoxamine, paroxetine, sertraline, N-acetylcysteine, serotonin reuptake inhibitors, epigallocatechin-3-galate (EGCG), diosgenin, prosapogenin III, quercetin, naringenin, curcumin, α-mangostin, rosmarinic acid, oxyresveratrol, apigenin derivatives, quinic acid derivatives, 6-shogaol, resveratrol, ginkgolide, limonoids, ginsenoside Rg3, berberine, galantamine, huperzine A, sophocarpidine, as well as compounds that inhibit the enzymatic degradation of PEA by targeting N-Acylethanolamine Acid Amidase (NAAA). Examples of NAAA inhibitors include F96 (Yang et al. (2015) Sci Rep. 5:13565), F215 (Zhou et al. (2019) Pharmacol Res. 145:104264; Li et al. (2018) Pharmacol Res. 132:7-14), ARN077 (Sasso et al. (2018) J Invest Dermatol. 138:562-569; Sasso et al. (2013) Pain 154:350-360), oxazolidone derivatives (Li et al. (2017) Eur J Med Chem. 139:214-221), and pyrrolidine amide derivatives (Zhou et al. (2018) Medchemcomm. 10:252-262). Additional compounds are also known in the art (Solorzano et al. (2009) Proc Natl Acad Sci U.S.A. 106:20966-20971; Ribeiro et al. (2015) ACS Chem Biol. 10:1838-1846; Migliore et al. (2016) Angew Chem Int Ed Engl. 55:11193-11197).

In some embodiments, the one or more additional therapeutic agents is selected from the group consisting of an immunosuppressive agent, a non-steroidal anti-inflammatory drug (NSAID), palmitoylethanolamide, an inhibitor of N-Acylethanolamine Acid Amidase (NAAA), interferon-β, glatiramer acetate, mitoxantrone, and glucocorticoids.

In some embodiments, the additional therapeutic agent is an immunotherapy agent. Immunotherapy refers to a treatment that modulates a subject’s immune system, e.g., checkpoint inhibitors, vaccines, cytokines, cell therapy, and dendritic cell therapy. Non-limiting examples of immunotherapies are checkpoint inhibitors include Nivolumab (BMS, anti-PD-1), Pembrolizumab (Merck, anti-PD-1), Ipilimumab (BMS, anti-CTLA-4), MEDI4736 (AstraZeneca, anti-PD-L1), and MPDL3280A (Roche, anti-PD-L1). Other immunotherapies may be vaccines, such as Gardail, Cervarix, BCG, sipulencel-T, Gp100:209-217, AGS-003, DCVax-L, Algenpantucel-L, Tergenpantucel-L, TG4010, ProstAtak, Prostvac-V/R-TRICOM, Rindopepimul, E75 peptide acetate, IMA901, POL-103A, Belagenpumatucel-L, GSK1572932A, MDX-1279, GV1001, and Tecemotide. The immunotherapy agent may be administered via injection (e.g., intravenously, subcutaneously, or into lymph nodes), but may also be administered orally, topically, or via aerosol. Immunotherapies may comprise adjuvants such as cytokines.

In some embodiments, the immunotherapy agent is an immune checkpoint inhibitor. Immune checkpoint inhibition broadly refers to inhibiting the checkpoints that prevent or downregulate an immune response. Examples of immune checkpoint proteins include, but are not limited to, CTLA4, PD-1, PD-L1, PD-L2, A2AR, B7-H3, B7-H4, BTLA, KIR, LAG3, TIM-3 or VISTA. Immune checkpoint inhibitors can be antibodies or antigen binding fragments thereof that bind to and inhibit an immune checkpoint protein. Examples of immune checkpoint inhibitors include, but are not limited to, nivolumab, pembrolizumab, pidilizumab, AMP-224, AMP-514, STI-A1110, TSR-042, RG-7446, BMS-936559, MEDI-4736, MSB-0010718C (avelumab), AUR-012 and STI-A1010. In some embodiments, the immune checkpoint inhibitor is a CTLA-4 inhibitor. In some embodiments, the immune checkpoint inhibitor is a PD-1 inhibitor. In some embodiments, the immune checkpoint inhibitor is a PD-L1 inhibitor. In some embodiments, the immune checkpoint inhibitor is an antibody.

In some embodiments, the methods provided herein include the administration of a pharmaceutical composition described herein in combination with one or more additional therapeutic agents. In some embodiments, the methods disclosed herein include the administration of two immunotherapy agents (e.g., immune checkpoint inhibitor). For example, the methods provided herein include the administration of a pharmaceutical composition described herein in combination with a PD-1 inhibitor (such as pemrolizumab or nivolumab or pidilizumab) or a CLTA-4 inhibitor (such as ipilimumab) or a PD-L1 inhibitor.

In some embodiments, the immunotherapy agent is an antibody or antigen binding fragment thereof that, for example, binds to a disease-associated antigen. Examples of disease-associated antigens include, but are not limited to, adipophilin, AIM-2, ALDH1A1, alpha-actinin-4, alpha-fetoprotein (“AFP”), ARTC1, B-RAF, BAGE-1, BCLX (L), BCR-ABL fusion protein b3a2, beta-catenin, BING-4, CA-125, CALCA, carcinoembryonic antigen (“CEA”), CASP-5, CASP-8, CD274, CD45, Cdc27, CDK12, CDK4, CDKN2A, CEA, CLPP, COA-1, CPSF, CSNK1A1, CTAG1, CTAG2, cyclin D1, Cyclin-A1, dek-can fusion protein, DKK1, EFTUD2, Elongation factor 2, ENAH (hMena), Ep-CAM, EpCAM, EphA3, epithelial tumor antigen (“ETA”), ETV6-AML1 fusion protein, EZH2, FGF5, FLT3-ITD, FN1, G250/MN/CAIX, GAGE-1,2,8, GAGE-3,4,5,6,7, GAS7, glypican-3, GnTV, gp100/Pmel17, GPNMB, HAUS3, Hepsin, HER-2/neu, HERV-K-MEL, HLA-A11, HLA-A2, HLA-DOB, hsp70-2, IDO1, IGF2B3, IL13Ralpha2, Intestinal carboxyl esterase, K-ras, Kallikrein 4, KIF20A, KK-LC-1, KKLC1, KM-HN-1, KMHN1 also known as CCDC110, LAGE-1, LDLR-fucosyltransferaseAS fusion protein, Lengsin, M-CSF, MAGE-A1, MAGE-A10, MAGE-A12, MAGE-A2, MAGE-A3, MAGE-A4, MAGE-A6, MAGE-A9, MAGE-C1, MAGE-C2, malic enzyme, mammaglobin-A, MART2, MATN, MC1R, MCSP, mdm-2, ME1, Melan-A/MART-1, Meloe, Midkine, MMP-2, MMP-7, MUC1, MUC5AC, mucin, MUM-1, MUM-2, MUM-3, Myosin, Myosin class I, N-raw, NA88-A, neo-PAP, NFYC, NY-BR-1, NY-ESO-1/LAGE-2, OA1, OGT, OS-9, P polypeptide, p53, PAP, PAX5, PBF, pml-RARalpha fusion protein, polymorphic epithelial mucin (“PEM”), PPP1R3B, PRAME, PRDX5, PSA, PSMA, PTPRK, RAB38/NY-MEL-1, RAGE-1, RBAF600, RGS5, RhoC, RNF43, RU2AS, SAGE, secernin 1, SIRT2, SNRPD1, SO×10, Sp17, SPA17, SSX-2, SSX-4, STEAP1, survivin, SYT-SSX1 or -SSX2 fusion protein, TAG-1, TAG-2, Telomerase, TGF-betaRII, TPBG, TRAG-3, Triosephosphate isomerase, TRP-1/gp75, TRP-2, TRP2-INT2, tyrosinase, tyrosinase (“TYR”), VEGF, WT1, XAGE-lb/GAGED2a. In some embodiments, the antigen is a neo-antigen.

In some embodiments, the immunotherapy agent is a vaccine and/or a component of a vaccine (e.g., an antigenic peptide and/or protein). The vaccine can be a protein vaccine, a nucleic acid vaccine or a combination thereof. For example, in some embodiments, the vaccine comprises a polypeptide comprising an epitope of a disease-associated antigen. In some embodiments, the vaccine comprises a nucleic acid (e.g., DNA or RNA, such as mRNA) that encodes an epitope of a disease-associated antigen. Examples of disease-associated antigens include, but are not limited to, adipophilin, AIM-2, ALDH1A1, alpha-actinin-4, alpha-fetoprotein (“AFP”), ARTC1, B-RAF, BAGE-1, BCLX (L), BCR-ABL fusion protein b3a2, beta-catenin, BING-4, CA-125, CALCA, carcinoembryonic antigen (“CEA”), CASP-5, CASP-8, CD274, CD45, Cdc27, CDK12, CDK4, CDKN2A, CEA, CLPP, COA-1, CPSF, CSNK1A1, CTAG1, CTAG2, cyclin D1, Cyclin-A1, dek-can fusion protein, DKK1, EFTUD2, Elongation factor 2, ENAH (hMena), Ep-CAM, EpCAM, EphA3, epithelial tumor antigen (“ETA”), ETV6-AML1 fusion protein, EZH2, FGF5, FLT3-ITD, FN1, G250/MN/CAIX, GAGE-1,2,8, GAGE-3,4,5,6,7, GAS7, glypican-3, GnTV, gp100/Pmel17, GPNMB, HAUS3, Hepsin, HER-2/neu, HERV-K-MEL, HLA-A11, HLA-A2, HLA-DOB, hsp70-2, IDO1, IGF2B3, IL13Ralpha2, Intestinal carboxyl esterase, K-ras, Kallikrein 4, KIF20A, KK-LC-1, KKLC1, KM-HN-1, KMHN1 also known as CCDC110, LAGE-1, LDLR-fucosyltransferaseAS fusion protein, Lengsin, M-CSF, MAGE-A1, MAGE-A10, MAGE-A12, MAGE-A2, MAGE-A3, MAGE-A4, MAGE-A6, MAGE-A9, MAGE-C1, MAGE-C2, malic enzyme, mammaglobin-A, MART2, MATN, MC1R, MCSP, mdm-2, ME1, Melan-A/MART-1, Meloe, Midkine, MMP-2, MMP-7, MUC1, MUC5AC, mucin, MUM-1, MUM-2, MUM-3, Myosin, Myosin class I, N-raw, NA88-A, neo-PAP, NFYC, NY-BR-1, NY-ESO-1/LAGE-2, OA1, OGT, OS-9, P polypeptide, p53, PAP, PAX5, PBF, pml-RARalpha fusion protein, polymorphic epithelial mucin (“PEM”), PPP1R3B, PRAME, PRDX5, PSA, PSMA, PTPRK, RAB38/NY-MEL-1, RAGE-1, RBAF600, RGS5, RhoC, RNF43, RU2AS, SAGE, secernin 1, SIRT2, SNRPD1, SO×10, Sp17, SPA17, SSX-2, SSX-4, STEAP1, survivin, SYT-SSX1 or -SSX2 fusion protein, TAG-1, TAG-2, Telomerase, TGF-betaRII, TPBG, TRAG-3, Triosephosphate isomerase, TRP-1/gp75, TRP-2, TRP2-INT2, tyrosinase, tyrosinase (“TYR”), VEGF, WT1, XAGE-lb/GAGED2a. In some embodiments, the antigen is a neo-antigen. In some embodiments, the vaccine is administered with an adjuvant. Examples of adjuvants include, but are not limited to, an immune modulatory protein, Adjuvant 65, α-GalCer, aluminum phosphate, aluminum hydroxide, calcium phosphate, β-Glucan Peptide, CpG ODN DNA, GPI-0100, lipid A, lipopolysaccharide, Lipovant, Montanide, N-acetyl-muramyl-L-alanyl-D-isoglutamine, Pam3CSK4, quil A, cholera toxin (CT) and heat-labile toxin from enterotoxigenic Escherichia coli (LT) including derivatives of these (CTB, mmCT, CTA1-DD, LTB, LTK63, LTR72, dmLT) and trehalose dimycolate.

In some embodiments, the immunotherapy agent is an immune modulating protein to the subject. In some embodiments, the immune modulatory protein is a cytokine or chemokine. Examples of immune modulating proteins include, but are not limited to, B lymphocyte chemoattractant (“BLC”), C-C motif chemokine 11 (“Eotaxin-1”), Eosinophil chemotactic protein 2 (“Eotaxin-2”), Granulocyte colony-stimulating factor (“G-CSF”), Granulocyte macrophage colony-stimulating factor (“GM-CSF”), 1-309, Intercellular Adhesion Molecule 1 (“ICAM-1”), Interferon alpha (“IFN-alpha”), Interferon beta (“IFN-beta”) Interferon gamma (“IFN-gamma”), Interlukin-1 alpha (“IL-1 alpha”), Interlukin-1 beta (“IL-1 beta”), Interleukin 1 receptor antagonist (“IL-1 ra”), Interleukin-2 (“IL-2”), Interleukin-4 (“IL-4”), Interleukin-5 (“IL-5”), Interleukin-6 (“IL-6”), Interleukin-6 soluble receptor (“IL-6 sR”), Interleukin-7 (“IL-7”), Interleukin-8 (“IL-8”), Interleukin- 10 (“IL-10”), Interleukin- 11 (“IL-11”), Subunit beta of Interleukin- 12 (“IL-12 p40” or “IL-12 p70”), Interleukin-13 (“IL-13”), Interleukin-15 (“IL-15”), Interleukin-16 (“IL-16”), Interleukin-17A-F (“IL-17A-F”), Interleukin-18 (“IL-18”), Interleukin-21 (“IL-21”), Interleukin-22 (“IL-22”), Interleukin-23 (“IL-23”), Interleukin-33 (“IL-33”), Chemokine (C-C motif) Ligand 2 (“MCP-1”), Macrophage colony-stimulating factor (“M-CSF”), Monokine induced by gamma interferon (“MIG”), Chemokine (C-C motif) ligand 2 (“MIP-1 alpha”), Chemokine (C-C motif) ligand 4 (“MIP-1 beta”), Macrophage inflammatory protein- 1 -delta (“MIP-1 delta”), Platelet-derived growth factor subunit B (“PDGF-BB”), Chemokine (C-C motif) ligand 5, Regulated on Activation, Normal T cell Expressed and Secreted (“RANTES”), TIMP metallopeptidase inhibitor 1 (“TIMP-1”), TIMP metallopeptidase inhibitor 2 (“TIMP-2”), Tumor necrosis factor, lymphotoxin-alpha (“TNF alpha”), Tumor necrosis factor, lymphotoxin-beta (“TNF beta”), Soluble TNF receptor type 1 (“sTNFRI”), sTNFRIIAR, Brain-derived neurotrophic factor (“BDNF”), Basic fibroblast growth factor (“bFGF”), Bone morphogenetic protein 4 (“BMP-4”), Bone morphogenetic protein 5 (“BMP-5”), Bone morphogenetic protein 7 (“BMP-7”), Nerve growth factor (“b-NGF”), Epidermal growth factor (“EGF”), Epidermal growth factor receptor (“EGFR”), Endocrine-gland-derived vascular endothelial growth factor (“EG-VEGF”), Fibroblast growth factor 4 (“FGF-4”), Keratinocyte growth factor (“FGF-7”), Growth differentiation factor 15 (“GDF-15”), Glial cell-derived neurotrophic factor (“GDNF”), Growth Hormone, Heparin-binding EGF-like growth factor (“HB-EGF”), Hepatocyte growth factor (“HGF”), Insulin-like growth factor binding protein 1 (“IGFBP-1”), Insulin-like growth factor binding protein 2 (“IGFBP-2”), Insulin-like growth factor binding protein 3 (“ IGFBP-3”), Insulin-like growth factor binding protein 4 (“IGFBP-4”), Insulin-like growth factor binding protein 6 (“IGFBP-6”), Insulin-like growth factor 1 (“IGF-1”), Insulin, Macrophage colony-stimulating factor (“M-CSF R”), Nerve growth factor receptor (“NGF R”), Neurotrophin-3 (“NT-3”), Neurotrophin-4 (“NT-4”), Osteoclastogenesis inhibitory factor (“Osteoprotegerin”), Platelet-derived growth factor receptors (“PDGF-AA”), Phosphatidylinositol-glycan biosynthesis (“PIGF”), Skp, Cullin, F-box containing comples (“SCF”), Stem cell factor receptor (“SCF R”), Transforming growth factor alpha (“TGFalpha”), Transforming growth factor beta-1 (“TGF beta 1”), Transforming growth factor beta-3 (“TGF beta 3”), Vascular endothelial growth factor (“VEGF”), Vascular endothelial growth factor receptor 2 (“VEGFR2”), Vascular endothelial growth factor receptor 3 (“VEGFR3”), VEGF-D 6Ckine, Tyrosine-protein kinase receptor UFO (“Axl”), Betacellulin (“BTC”), Mucosae-associated epithelial chemokine (“CCL28”), Chemokine C-C motif) ligand 27 (“CTACK”), Chemokine (C-X-C motif) ligand 16 (“CXCL16”), C-X-C motif chemokine 5 (“ENA-78”), Chemokine (C-C motif) ligand 26 (“Eotaxin-3”), Granulocyte chemotactic protein 2 (“GCP-2”), GRO, Chemokine (C-C motif) ligand 14 (“HCC-1”), Chemokine (C-C motif) ligand 16 (“HCC-4”), Interleukin-9 (“IL-9”), Interleukin-17 F (“IL-17F”), Interleukin- 18-binding protein (“IL-18 BPa”), Interleukin-28 A (“IL-28A”), Interleukin 29 (“IL-29”), Interleukin 31 (“IL-31”), C-X-C motif chemokine 10 (“IP-10”), Chemokine receptor CXCR3 (“I-TAC”), Leukemia inhibitory factor (”LIF”), Light, Chemokine (C motif) ligand (“Lymphotactin”), Monocyte chemoattractant protein 2 (“MCP-2”), Monocyte chemoattractant protein 3 (“MCP-3”), Monocyte chemoattractant protein 4 (“MCP-4”), Macrophage-derived chemokine (“MDC”), Macrophage migration inhibitory factor (“MIF”), Chemokine (C-C motif) ligand 20 (“MIP-3 alpha”), C-C motif chemokine 19 (“MIP-3 beta”), Chemokine (C-C motif) ligand 23 (“MPIF-1”), Macrophage stimulating protein alpha chain (“MSPalpha”), Nucleosome assembly protein 1-like 4 (“NAP-2”), Secreted phosphoprotein 1 (“Osteopontin”), Pulmonary and activation-regulated cytokine (“PARC”), Platelet factor 4 (“PF4”), Stroma cell-derived factor- 1 alpha (“SDF-1 alpha”), Chemokine (C-C motif) ligand 17 (“TARC”), Thymus-expressed chemokine (“TECK”), Thymic stromal lymphopoietin (“TSLP 4- IBB”), CD 166 antigen (“ALCAM”), Cluster of Differentiation 80 (“B7-1”), Tumor necrosis factor receptor superfamily member 17 (“BCMA”), Cluster of Differentiation 14 (“CD14”), Cluster of Differentiation 30 (“CD30”), Cluster of Differentiation 40 (“CD40 Ligand”), Carcinoembryonic antigen-related cell adhesion molecule 1 (biliary glycoprotein) (“CEACAM-1”), Death Receptor 6 (“DR6”), Deoxythymidine kinase (“Dtk”), Type 1 membrane glycoprotein (“Endoglin”), Receptor tyrosine-protein kinase erbB-3 (“ErbB3”), Endothelial-leukocyte adhesion molecule 1 (“E-Selectin”), Apoptosis antigen 1 (“Fas”), Fms-like tyrosine kinase 3 (“Flt-3L”), Tumor necrosis factor receptor superfamily member 1 (“GITR”), Tumor necrosis factor receptor superfamily member 14 (“HVEM”), Intercellular adhesion molecule 3 (“ICAM-3”), IL-1 R4, IL-1 RI, IL-10 Rbeta, IL-17R, IL-2Rgamma, IL-21R, Lysosome membrane protein 2 (“LIMPII”), Neutrophil gelatinase-associated lipocalin (“Lipocalin-2”), CD62L (“L-Selectin”), Lymphatic endothelium (“LYVE-1”), MHC class I polypeptide-related sequence A (“MICA”), MHC class I polypeptide-related sequence B (“MICB”), NRGl-betal, Beta-type platelet-derived growth factor receptor (“PDGF Rbeta”), Platelet endothelial cell adhesion molecule (“PECAM-1”), RAGE, Hepatitis A virus cellular receptor 1 (“TIM-1”), Tumor necrosis factor receptor superfamily member IOC (“TRAIL R3”), Trappin protein transglutaminase binding domain (“Trappin-2”), Urokinase receptor (“uPAR”), Vascular cell adhesion protein 1 (“VCAM-1”), XEDARActivin A, Agouti-related protein (“AgRP”), Ribonuclease 5 (“Angiogenin”), Angiopoietin 1, Angiostatin, Catheprin S, CD40, Cryptic family protein IB (“Cripto-1”), DAN, Dickkopf-related protein 1 (“DKK-1”), E-Cadherin, Epithelial cell adhesion molecule (“EpCAM”), Fas Ligand (FasL or CD95L), Fcg RIIB/C, Follistatin, Galectin-7, Intercellular adhesion molecule 2 (“ICAM-2”), IL-13 R1, IL-13R2, IL-17B, IL-2 Ra, IL-2 Rb, IL-23, LAP, Neuronal cell adhesion molecule (“NrCAM”), Plasminogen activator inhibitor- 1 (“PAI-1”), Platelet derived growth factor receptors (“PDGF-AB”), Resistin, stromal cell-derived factor 1 (“SDF-1 beta”), sgpl30, Secreted frizzled-related protein 2 (“ShhN”), Sialic acid-binding immunoglobulin-type lectins (“Siglec-5”), ST2, Transforming growth factor-beta 2 (“TGF beta 2”), Tie-2, Thrombopoietin (“TPO”), Tumor necrosis factor receptor superfamily member 10D (“TRAIL R4”), Triggering receptor expressed on myeloid cells 1 (“TREM-1”), Vascular endothelial growth factor C (“VEGF-C”), VEGFRlAdiponectin, Adipsin (“AND”), Alpha-fetoprotein (“AFP”), Angiopoietin-like 4 (“ANGPTL4”), Beta-2-microglobulin (“B2M”), Basal cell adhesion molecule (“BCAM”), Carbohydrate antigen 125 (“CA125”), Cancer Antigen 15-3 (“CA15-3”), Carcinoembryonic antigen (“CEA”), cAMP receptor protein (“CRP”), Human Epidermal Growth Factor Receptor 2 (“ErbB2”), Follistatin, Follicle-stimulating hormone (“FSH”), Chemokine (C-X-C motif) ligand 1 (“GRO alpha”), human chorionic gonadotropin (“beta HCG”), Insulin-like growth factor 1 receptor (“IGF-1 sR”), IL-1 sRII, IL-3, IL-18 Rb, IL-21, Leptin, Matrix metalloproteinase-1 (“MMP-1”), Matrix metalloproteinase-2 (“MMP-2”), Matrix metalloproteinase-3 (“MMP-3”), Matrix metalloproteinase-8 (“MMP-8”), Matrix metalloproteinase-9 (“MMP-9”), Matrix metalloproteinase-10 (“MMP-10”), Matrix metalloproteinase-13 (“MMP-13”), Neural Cell Adhesion Molecule (“NCAM-1”), Entactin (“Nidogen-1”), Neuron specific enolase (“NSE”), Oncostatin M (“OSM”), Procalcitonin, Prolactin, Prostate specific antigen (“PSA”), Sialic acid-binding Ig-like lectin 9 (“Siglec-9”), ADAM 17 endopeptidase (“TACE”), Thyroglobulin, Metalloproteinase inhibitor 4 (“TIMP-4”), TSH2B4, Disintegrin and metalloproteinase domain-containing protein 9 (“ADAM-9”), Angiopoietin 2, Tumor necrosis factor ligand superfamily member 13/ Acidic leucine-rich nuclear phosphoprotein 32 family member B (“APRIL”), Bone morphogenetic protein 2 (“BMP-2”), Bone morphogenetic protein 9 (“BMP-9”), Complement component 5a (“C5a”), Cathepsin L, CD200, CD97, Chemerin, Tumor necrosis factor receptor superfamily member 6B (“DcR3”), Fatty acid-binding protein 2 (“FABP2”), Fibroblast activation protein, alpha (“FAP”), Fibroblast growth factor 19 (“FGF-19”), Galectin-3, Hepatocyte growth factor receptor (“HGF R”), IFN-gammalpha/beta R2, Insulin-like growth factor 2 (“IGF-2”), Insulin-like growth factor 2 receptor (“IGF-2 R”), Interleukin-1 receptor 6 (“IL-1R6”), Interleukin 24 (“IL-24”), Interleukin 33 (“IL-33”, Kallikrein 14, Asparaginyl endopeptidase (“Legumain”), Oxidized low-density lipoprotein receptor 1 (“LOX-1”), Mannose-binding lectin (“MBL”), Neprilysin (“NEP”), Notch homolog 1, translocation-associated (Drosophila) (“Notch-1”), Nephroblastoma overexpressed (“NOV”), Osteoactivin, Programmed cell death protein 1 (“PD-1”), N-acetylmuramoyl-L-alanine amidase (“PGRP-5”), Serpin A4, Secreted frizzled related protein 3 (“sFRP-3”), Thrombomodulin, Tolllike receptor 2 (“TLR2”), Tumor necrosis factor receptor superfamily member 10A (“TRAIL Rl”), Transferrin (“TRF”), WIF-lACE-2, Albumin, AMICA, Angiopoietin 4, B-cell activating factor (“BAFF”), Carbohydrate antigen 19-9 (“CA19-9”), CD 163, Clusterin, CRT AM, Chemokine (C-X-C motif) ligand 14 (“CXCL14”), Cystatin C, Decorin (“DCN”), Dickkopf-related protein 3 (“Dkk-3”), Delta-like protein 1 (“DLL1”), Fetuin A, Heparin-binding growth factor 1 (“aFGF”), Folate receptor alpha (“FOLR1”), Furin, GPCR-associated sorting protein 1 (“GASP-1”), GPCR-associated sorting protein 2 (“GASP-2”), Granulocyte colony-stimulating factor receptor (“GCSF R”), Serine protease hepsin (“HAI-2”), Interleukin-17B Receptor (“IL-17B R”), Interleukin 27 (“IL-27”), Lymphocyte-activation gene 3 (“LAG-3”), Apolipoprotein A-V (“LDL R”), Pepsinogen I, Retinol binding protein 4 (“RBP4”), SOST, Heparan sulfate proteoglycan (“Syndecan-1”), Tumor necrosis factor receptor superfamily member 13B (“TACI”), Tissue factor pathway inhibitor (“TFPI”), TSP-1, Tumor necrosis factor receptor superfamily, member 10b (“TRAIL R2”), TRANCE, Troponin I, Urokinase Plasminogen Activator (“uPA”), Cadherin 5, type 2 or VE-cadherin (vascular endothelial) also known as CD144 (“VE-Cadherin”), WNTl-inducible-signaling pathway protein 1 (“WISP-1”), and Receptor Activator of Nuclear Factor κ B (“RANK”).

In some embodiments, the additional therapeutic agent is an immunosuppressive agent, a DMARD, a pain-control drug, a steroid, a non-steroidal anti-inflammatory drug (NSAID), a cytokine antagonist, cyclosporin, retinoids, corticosteroids, propionic acid derivative, acetic acid derivative, enolic acid derivatives, fenamic acid derivatives, Cox-2 inhibitors, lumiracoxib, ibuprofen, cholin magnesium salicylate, fenoprofen, salsalate, difunisal, tolmetin, ketoprofen, flurbiprofen, oxaprozin, indomethacin, sulindac, etodolac, ketorolac, nabumetone, naproxen, valdecoxib, etoricoxib, MK0966; rofecoxib, acetaminophen, Celecoxib, Diclofenac, tramadol, piroxicam, meloxicam, tenoxicam, droxicam, lornoxicam, isoxicam, mefanamic acid, meclofenamic acid, flufenamic acid, tolfenamic, valdecoxib, parecoxib, etodolac, indomethacin, aspirin, ibuprophen, firocoxib, methotrexate (MTX), antimalarial drugs, hydroxychloroquine, chloroquine, sulfasalazine, Leflunomide, azathioprine, cyclosporin, gold salts, minocycline, cyclophosphamide, D-penicillamine, minocycline, auranofin, tacrolimus, myocrisin, chlorambucil, TNF alpha antagonists, TNF alpha antagonists, TNF alpha receptor antagonists, ADALIMUMAB (Humira®), ETANERCEPT (Enbrel®), INFLIXIMAB (Remicade®; TA-650), CERTOLIZUMAB PEGOL (Cimzia®; CDP870), GOLIMUMAB (Simpom®; CNTO 148), ANAKINRA (Kineret®), RITUXIMAB (Rituxan®; MabThera®), ABATACEPT (Orencia®), TOCILIZUMAB (RoActemra /Actemra®), integrin antagonists, TYSABRI® (natalizumab), IL-1 antagonists, ACZ885 (Ilaris), Anakinra (Kineret®), CD4 antagonists, IL-23 antagonists, IL-20 antagonists, IL-6 antagonists, BLyS antagonists, Atacicept, Benlysta®/ LymphoStat-B® (belimumab), p38 Inhibitors, CD20 antagonists, Ocrelizumab, Ofatumumab (Arzerra®), interferon gamma antagonists, Fontolizumab, prednisolone, Prednisone, dexamethasone, Cortisol, cortisone, hydrocortisone, methylprednisolone, betamethasone, triamcinolone, beclometasome, fludrocortisone, deoxycorticosterone, aldosterone, Doxycycline, vancomycin, pioglitazone, SBI-087, SCIO-469, Cura-100, Oncoxin + Viusid, TwHF, Methoxsalen, Vitamin D - ergocalciferol, Milnacipran, Paclitaxel, rosig tazone, Tacrolimus, Prograf®, RADOOl, rapamune, rapamycin, fostamatinib, Fentanyl, XOMA 052, Fostamatinib disodium, rosightazone, Curcumin, Longvida™, Rosuvastatin, Maraviroc, ramipnl, Milnacipran, Cobiprostone, somatropin, tgAAC94 gene therapy vector, MK0359, GW856553, esomeprazole, everolimus, trastuzumab, JAKl and JAK2 inhibitors, pan JAK inhibitors, e.g., tetracyclic pyridone 6 (P6), 325, PF-956980, denosumab, IL-6 antagonists, CD20 antagonistis, CTLA4 antagonists, IL-8 antagonists, IL-21 antagonists, IL-22 antagonist, integrin antagonists, Tysarbri® (natalizumab), VGEF antagnosits, CXCL antagonists, MMP antagonists, defensin antagonists, IL-1 antagonists, IL-1 beta antagonsits, IL-23 antagonists, receptor decoys, antagonistic antibodies, corticosteroids, mesalazine, mesalamine, sulfasalazine, sulfasalazine derivatives, immunosuppressive drugs, cyclosporin A, mercaptopurine, azathiopurine, prednisone, methotrexate, antihistamines, glucocorticoids, epinephrine, theophylline, cromolyn sodium, anti-leukotrienes, anti-cholinergic drugs for rhinitis, TLR antagonists, inflammasome inhibitors, anti-cholinergic decongestants, mast-cell stabilizers, monoclonal anti-IgE antibodies, vaccines, cytokine inhibitors, anti-IL-6 antibodies, TNF inhibitors, palmitoylethanolamide, an inhibitor of N-Acylethanolamine Acid Amidase (NAAA), interferon-β, glatiramer acetate, mitoxantrone, and glucocorticoids.

In some embodiments, the neuroinflammatory disorder therapy comprises administering a therapeutic bacteria and/or a therapeutic combination of bacteria to the subject so a healthy microbiome can be reconstituted in the subject. In some embodiments, therapeutic bacteria is a non-immune-disorder-associated bacteria. In some embodiments therapeutic bacteria is a probiotic bacteria.

In some embodiments, the additional therapeutic agent is an antibiotic. For example, if the presence of a disease-associated bacteria and/or a disease-associated microbiome profile is detected according to the methods provided herein, antibiotics can be administered to eliminate the disease-associated bacteria from the subject. “Antibiotics” broadly refers to compounds capable of inhibiting or preventing a bacterial infection. Antibiotics can be classified in a number of ways, including their use for specific infections, their mechanism of action, their bioavailability, or their spectrum of target microbe (e.g., Gram-negative vs. Gram-positive bacteria, aerobic vs. anaerobic bacteria, etc.) and these may be used to kill specific bacteria in specific areas of the host (“niches”) (Leekha, et al 2011. General Principles of Antimicrobial Therapy. Mayo Clin Proc. 86(2): 156-167). In certain embodiments, antibiotics can be used to selectively target bacteria of a specific niche. In some embodiments, antibiotics known to treat a particular infection that includes a disease niche may be used to target disease-associated microbes, including disease-associated bacteria in that niche. In other embodiments, antibiotics are administered after the pharmaceutical composition comprising mEVs (such as smEVs and/or pmEVs), bacteria, or any combination thereof. In some embodiments, antibiotics are administered before pharmaceutical composition comprising mEVs (such as smEVs and/or pmEVs), bacteria, or any combination thereof.

In some aspects, antibiotics can be selected based on their bactericidal or bacteriostatic properties. Bactericidal antibiotics include mechanisms of action that disrupt the cell wall (e.g., β-lactams), the cell membrane (e.g., daptomycin), or bacterial DNA (e.g., fluoroquinolones). Bacteriostatic agents inhibit bacterial replication and include sulfonamides, tetracyclines, and macrolides, and act by inhibiting protein synthesis. Furthermore, while some drugs can be bactericidal in certain organisms and bacteriostatic in others, knowing the target organism allows one skilled in the art to select an antibiotic with the appropriate properties. In certain treatment conditions, bacteriostatic antibiotics inhibit the activity of bactericidal antibiotics. Thus, in certain embodiments, bactericidal and bacteriostatic antibiotics are not combined.

Antibiotics include, but are not limited to aminoglycosides, ansamycins, carbacephems, carbapenems, cephalosporins, glycopeptides, lincosamides, lipopeptides, macrolides, monobactams, nitrofurans, oxazolidonones, penicillins, polypeptide antibiotics, quinolones, fluoroquinolone, sulfonamides, tetracyclines, and anti-mycobacterial compounds, and combinations thereof.

Aminoglycosides include, but are not limited to Amikacin, Gentamicin, Kanamycin, Neomycin, Netilmicin, Tobramycin, Paromomycin, and Spectinomycin. Aminoglycosides are effective, e.g., against Gram-negative bacteria, such as Escherichia coli, Klebsiella, Pseudomonas aeruginosa, and Francisella tularensis, and against certain aerobic bacteria but less effective against obligate/facultative anaerobes. Aminoglycosides are believed to bind to the bacterial 30S or 50S ribosomal subunit thereby inhibiting bacterial protein synthesis.

Ansamycins include, but are not limited to, Geldanamycin, Herbimycin, Rifamycin, and Streptovaricin. Geldanamycin and Herbimycin are believed to inhibit or alter the function of Heat Shock Protein 90.

Carbacephems include, but are not limited to, Loracarbef. Carbacephems are believed to inhibit bacterial cell wall synthesis.

Carbapenems include, but are not limited to, Ertapenem, Doripenem, Imipenem/Cilastatin, and Meropenem. Carbapenems are bactericidal for both Gram-positive and Gram-negative bacteria as broad-spectrum antibiotics. Carbapenems are believed to inhibit bacterial cell wall synthesis.

Cephalosporins include, but are not limited to, Cefadroxil, Cefazolin, Cefalotin, Cefalothin, Cefalexin, Cefaclor, Cefamandole, Cefoxitin, Cefprozil, Cefuroxime, Cefixime, Cefdinir, Cefditoren, Cefoperazone, Cefotaxime, Cefpodoxime, Ceftazidime, Ceftibuten, Ceftizoxime, Ceftriaxone, Cefepime, Ceftaroline fosamil,and Ceftobiprole. Selected Cephalosporins are effective, e.g., against Gram-negative bacteria and against Gram-positive bacteria, including Pseudomonas, certain Cephalosporins are effective against methicillin-resistant Staphylococcus aureus (MRSA). Cephalosporins are believed to inhibit bacterial cell wall synthesis by disrupting synthesis of the peptidoglycan layer of bacterial cell walls.

Glycopeptides include, but are not limited to, Teicoplanin, Vancomycin, and Telavancin. Glycopeptides are effective, e.g., against aerobic and anaerobic Gram-positive bacteria including MRSA and Clostridium difficile. Glycopeptides are believed to inhibit bacterial cell wall synthesis by disrupting synthesis of the peptidoglycan layer of bacterial cell walls.

Lincosamides include, but are not limited to, Clindamycin and Lincomycin. Lincosamides are effective, e.g., against anaerobic bacteria, as well as Staphylococcus, and Streptococcus. Lincosamides are believed to bind to the bacterial 50S ribosomal subunit thereby inhibiting bacterial protein synthesis.

Lipopeptides include, but are not limited to, Daptomycin. Lipopeptides are effective, e.g., against Gram-positive bacteria. Lipopeptides are believed to bind to the bacterial membrane and cause rapid depolarization.

Macrolides include, but are not limited to, Azithromycin, Clarithromycin, Dirithromycin, Erythromycin, Roxithromycin, Troleandomycin, Telithromycin, and Spiramycin. Macrolides are effective, e.g., against Streptococcus and Mycoplasma. Macrolides are believed to bind to the bacterial or 50S ribosomal subunit, thereby inhibiting bacterial protein synthesis.

Monobactams include, but are not limited to, Aztreonam. Monobactams are effective, e.g., against Gram-negative bacteria. Monobactams are believed to inhibit bacterial cell wall synthesis by disrupting synthesis of the peptidoglycan layer of bacterial cell walls.

Nitrofurans include, but are not limited to, Furazolidone and Nitrofurantoin.

Oxazolidonones include, but are not limited to, Linezolid, Posizolid, Radezolid, and Torezolid. Oxazolidonones are believed to be protein synthesis inhibitors.

Penicillins include, but are not limited to, Amoxicillin, Ampicillin, Azlocillin, Carbenicillin, Cloxacillin, Dicloxacillin, Flucloxacillin, Mezlocillin, Methicillin, Nafcillin, Oxacillin, Penicillin G, Penicillin V, Piperacillin, Temocillin and Ticarcillin. Penicillins are effective, e.g., against Gram-positive bacteria, facultative anaerobes, e.g., Streptococcus, Borrelia, and Treponema. Penicillins are believed to inhibit bacterial cell wall synthesis by disrupting synthesis of the peptidoglycan layer of bacterial cell walls.

Penicillin combinations include, but are not limited to, Amoxicillin/clavulanate, Ampicillin/sulbactam, Piperacillin/tazobactam, and Ticarcillin/clavulanate.

Polypeptide antibiotics include, but are not limited to, Bacitracin, Colistin, and Polymyxin B and E. Polypeptide Antibiotics are effective, e.g., against Gram-negative bacteria. Certain polypeptide antibiotics are believed to inhibit isoprenyl pyrophosphate involved in synthesis of the peptidoglycan layer of bacterial cell walls, while others destabilize the bacterial outer membrane by displacing bacterial counter-ions.

Quinolones and Fluoroquinolone include, but are not limited to, Ciprofloxacin, Enoxacin, Gatifloxacin, Gemifloxacin, Levofloxacin, Lomefloxacin, Moxifloxacin, Nalidixic acid, Norfloxacin, Ofloxacin, Trovafloxacin, Grepafloxacin, Sparfloxacin, and Temafloxacin. Quinolones/Fluoroquinolone are effective, e.g., against Streptococcus and Neisseria. Quinolones/Fluoroquinolone are believed to inhibit the bacterial DNA gyrase or topoisomerase IV, thereby inhibiting DNA replication and transcription.

Sulfonamides include, but are not limited to, Mafenide, Sulfacetamide, Sulfadiazine, Silver sulfadiazine, Sulfadimethoxine, Sulfamethizole, Sulfamethoxazole, Sulfanilimide, Sulfasalazine, Sulfisoxazole, Trimethoprim-Sulfamethoxazole (Co-trimoxazole), and Sulfonamidochrysoidine. Sulfonamides are believed to inhibit folate synthesis by competitive inhibition of dihydropteroate synthetase, thereby inhibiting nucleic acid synthesis.

Tetracyclines include, but are not limited to, Demeclocycline, Doxycycline, Minocycline, Oxytetracycline, and Tetracycline. Tetracyclines are effective, e.g., against Gram-negative bacteria. Tetracyclines are believed to bind to the bacterial 30S ribosomal subunit thereby inhibiting bacterial protein synthesis.

Anti-mycobacterial compounds include, but are not limited to, Clofazimine, Dapsone, Capreomycin, Cycloserine, Ethambutol, Ethionamide, Isoniazid, Pyrazinamide, Rifampicin, Rifabutin, Rifapentine, and Streptomycin.

Suitable antibiotics also include arsphenamine, chloramphenicol, fosfomycin, fusidic acid, metronidazole, mupirocin, platensimycin, quinupristin/dalfopristin, tigecycline, tinidazole, trimethoprim amoxicillin/clavulanate, ampicillin/sulbactam, amphomycin ristocetin, azithromycin, bacitracin, buforin II, carbomycin, cecropin Pl, clarithromycin, erythromycins, furazolidone, fusidic acid, Na fusidate, gramicidin, imipenem, indolicidin, josamycin, magainan II, metronidazole, nitroimidazoles, mikamycin, mutacin B-Ny266, mutacin B-JHl 140, mutacin J-T8, nisin, nisin A, novobiocin, oleandomycin, ostreogrycin, piperacillin/tazobactam, pristinamycin, ramoplanin, ranalexin, reuterin, rifaximin, rosamicin, rosaramicin, spectinomycin, spiramycin, staphylomycin, streptogramin, streptogramin A, synergistin, taurolidine, teicoplanin, telithromycin, ticarcillin/clavulanic acid, triacetyloleandomycin, tylosin, tyrocidin, tyrothricin, vancomycin, vemamycin, and virginiamycin.

### Administration

In certain aspects, provided herein is a method of delivering a pharmaceutical composition described herein (e.g., a pharmaceutical composition comprising Veillonella parvula (e.g., Veillonella parvula Strain A) mEVs (such as smEVs and/or pmEVs), bacteria, or any combination thereof) to a subject. In some embodiments of the methods provided herein, the pharmaceutical composition is administered in conjunction with the administration of an additional therapeutic agent. In some embodiments, the pharmaceutical composition comprises mEVs (such as smEVs and/or pmEVs), bacteria, or any combination thereof, co-formulated with the additional therapeutic agent. In some embodiments, the pharmaceutical composition comprising mEVs (such as smEVs and/or pmEVs), bacteria, or any combination thereof, is co-administered with the additional therapeutic agent. In some embodiments, the additional therapeutic agent is administered to the subject before administration of the pharmaceutical composition that comprises mEVs (such as smEVs and/or pmEVs), bacteria, or any combination thereof (e.g., about 1, 2, 3, 4, 5, 6, 7, 8, 9, 10, 15, 20, 25, 30, 35, 40, 45, 50 or 55 minutes before, about 1, 2, 3, 4, 5, 6, 7, 8, 9, 10, 11, 12, 13, 14, 15, 16, 17, 18, 19, 20, 21, 22 or 23 hours before, or about 1, 2, 3, 4, 5, 6, 7, 8, 9, 10, 11, 12, 13 or 14 days before). In some embodiments, the additional therapeutic agent is administered to the subject after administration of the pharmaceutical composition that comprises mEVs (such as smEVs and/or pmEVs), bacteria, or any combination thereof (e.g., about 1, 2, 3, 4, 5, 6, 7, 8, 9, 10, 15, 20, 25, 30, 35, 40, 45, 50 or 55 minutes after, about 1, 2, 3, 4, 5, 6, 7, 8, 9, 10, 11, 12, 13, 14, 15, 16, 17, 18, 19, 20, 21, 22 or 23 hours after, or about 1, 2, 3, 4, 5, 6, 7, 8, 9, 10, 11, 12, 13 or 14 days after). In some embodiments, the same mode of delivery is used to deliver both the pharmaceutical composition that comprises mEVs (such as smEVs and/or pmEVs), bacteria, or any combination thereof, and the additional therapeutic agent. In some embodiments, different modes of delivery are used to administer the pharmaceutical composition that comprises mEVs (such as smEVs and/or pmEVs), bacteria, or any combination thereof, and the additional therapeutic agent. For example, in some embodiments the pharmaceutical composition that comprises mEVs (such as smEVs and/or pmEVs), bacteria, or any combination thereof, is administered orally while the additional therapeutic agent is administered via injection (e.g., an intravenous or intramuscular and injection). In some embodiments, the pharmaceutical composition described herein is administered once a day. In some embodiments, the pharmaceutical composition described herein is administered twice a day. In some embodiments, the pharmaceutical composition described herein is formulated for a daily dose. In some embodiments, the pharmaceutical composition described herein is formulated for twice a day dose, wherein each dose is half of the daily dose.

In certain embodiments, the pharmaceutical compositions and dosage forms described herein can be administered in conjunction with any other conventional immunotherapy treatment. These treatments may be applied as necessary and/or as indicated and may occur before, concurrent with or after administration of the pharmaceutical composition that comprises mEVs (such as smEVs and/or pmEVs), bacteria, or any combination thereof, or dosage forms described herein.

The dosage regimen can be any of a variety of methods and amounts, and can be determined by one skilled in the art according to known clinical factors. As is known in the medical arts, dosages for any one patient can depend on many factors, including the subject’s species, size, body surface area, age, sex, immunocompetence, and general health, the particular microorganism to be administered, duration and route of administration, the kind and stage of the disease, and other compounds such as drugs being administered concurrently or near-concurrently. In addition to the above factors, such levels can be affected by the infectivity of the microorganism, and the nature of the microorganism, as can be determined by one skilled in the art. In the present methods, appropriate minimum dosage levels of microorganisms can be levels sufficient for the microorganism to survive, grow and replicate. The dose of a pharmaceutical composition that comprises mEVs (such as smEVs and/or pmEVs), bacteria, or any combination thereof, described herein may be appropriately set or adjusted in accordance with the dosage form, the route of administration, the degree or stage of a target disease, and the like. For example, the general effective dose of the agents may range between 0.01 mg/kg body weight/day and 1000 mg/kg body weight/day, between 0.1 mg/kg body weight/day and 1000 mg/kg body weight/day, 0.5 mg/kg body weight/day and 500 mg/kg body weight/day, 1 mg/kg body weight/day and 100 mg/kg body weight/day, or between 5 mg/kg body weight/day and 50 mg/kg body weight/day. The effective dose may be 0.01, 0.05, 0.1, 0.5, 1, 2, 3, 5, 10, 20, 30, 40, 50, 60, 70, 80, 90, 100, 200, 500, or 1000 mg/kg body weight/day or more, but the dose is not limited thereto.

In some embodiments, the dose administered to a subject is sufficient to prevent disease (e.g., a neuroinflammatory disease, a neurodegenerative disease, a neuromuscular disease, and/or a psychiatric disorder), delay its onset, or slow or stop its progression, or relieve one or more symptoms of the disease. One skilled in the art will recognize that dosage will depend upon a variety of factors including the strength of the particular agent (e.g., therapeutic agent) employed, as well as the age, species, condition, and body weight of the subject. The size of the dose will also be determined by the route, timing, and frequency of administration as well as the existence, nature, and extent of any adverse side-effects that might accompany the administration of a particular therapeutic agent and the desired physiological effect.

Suitable doses and dosage regimens can be determined by conventional range-finding techniques known to those of ordinary skill in the art. Generally, treatment is initiated with smaller dosages, which are less than the optimum dose of the compound. Thereafter, the dosage is increased by small increments until the optimum effect under the circumstances is reached. An effective dosage and treatment protocol can be determined by routine and conventional means, starting e.g., with a low dose in laboratory animals and then increasing the dosage while monitoring the effects, and systematically varying the dosage regimen as well. Animal studies are commonly used to determine the maximal tolerable dose (“MTD”) of bioactive agent per kilogram weight. Those skilled in the art regularly extrapolate doses for efficacy, while avoiding toxicity, in other species, including humans.

In accordance with the above, in therapeutic applications, the dosages of the therapeutic agents used in accordance with the invention vary depending on the active agent, the age, weight, and clinical condition of the recipient patient, and the experience and judgment of the clinician or practitioner administering the therapy, among other factors affecting the selected dosage. For example, the dose should be sufficient to result in slowing of progression of the disease for which the subject is being treated, and preferably amelioration of one or more symptoms of the disease for which the subject is being treated.

Separate administrations can include any number of two or more administrations, including two, three, four, five or six administrations. One skilled in the art can readily determine the number of administrations to perform or the desirability of performing one or more additional administrations according to methods known in the art for monitoring therapeutic methods and other monitoring methods provided herein. Accordingly, the methods provided herein include methods of providing to the subject one or more administrations of a pharmaceutical composition, where the number of administrations can be determined by monitoring the subject, and, based on the results of the monitoring, determining whether or not to provide one or more additional administrations. Deciding on whether or not to provide one or more additional administrations can be based on a variety of monitoring results.

The time period between administrations can be any of a variety of time periods. The time period between administrations can be a function of any of a variety of factors, including monitoring steps, as described in relation to the number of administrations, the time period for a subject to mount an immune response. In one example, the time period can be a function of the time period for a subject to mount an immune response; for example, the time period can be more than the time period for a subject to mount an immune response, such as more than about one week, more than about ten days, more than about two weeks, or more than about a month; in another example, the time period can be less than the time period for a subject to mount an immune response, such as less than about one week, less than about ten days, less than about two weeks, or less than about a month.

In some embodiments, the delivery of an additional therapeutic agent in combination with the pharmaceutical composition described herein reduces the adverse effects and/or improves the efficacy of the additional therapeutic agent.

The effective dose of an additional therapeutic agent described herein is the amount of the additional therapeutic agent that is effective to achieve the desired therapeutic response for a particular subject, composition, and mode of administration, with the least toxicity to the subject. The effective dosage level can be identified using the methods described herein and will depend upon a variety of pharmacokinetic factors including the activity of the particular compositions or agents administered, the route of administration, the time of administration, the rate of excretion of the particular compound being employed, the duration of the treatment, other drugs, compounds and/or materials used in combination with the particular compositions employed, the age, sex, weight, condition, general health and prior medical history of the subject being treated, and like factors well known in the medical arts. In general, an effective dose of an additional therapeutic agent will be the amount of the additional therapeutic agent which is the lowest dose effective to produce a therapeutic effect. Such an effective dose will generally depend upon the factors described above.

The toxicity of an additional therapeutic agent is the level of adverse effects experienced by the subject during and following treatment. Adverse events associated with additional therapy toxicity can include, but are not limited to, abdominal pain, acid indigestion, acid reflux, allergic reactions, alopecia, anaphylasix, anemia, anxiety, lack of appetite, arthralgias, asthenia, ataxia, azotemia, loss of balance, bone pain, bleeding, blood clots, low blood pressure, elevated blood pressure, difficulty breathing, bronchitis, bruising, low white blood cell count, low red blood cell count, low platelet count, cardiotoxicity, cystitis, hemorrhagic cystitis, arrhythmias, heart valve disease, cardiomyopathy, coronary artery disease, cataracts, central neurotoxicity, cognitive impairment, confusion, conjunctivitis, constipation, coughing, cramping, cystitis, deep vein thrombosis, dehydration, depression, diarrhea, dizziness, dry mouth, dry skin, dyspepsia, dyspnea, edema, electrolyte imbalance, esophagitis, fatigue, loss of fertility, fever, flatulence, flushing, gastric reflux, gastroesophageal reflux disease, genital pain, granulocytopenia, gynecomastia, glaucoma, hair loss, hand-foot syndrome, headache, hearing loss, heart failure, heart palpitations, heartburn, hematoma, hemorrhagic cystitis, hepatotoxicity, hyperamylasemia, hypercalcemia, hyperchloremia, hyperglycemia, hyperkalemia, hyperlipasemia, hypermagnesemia, hypematremia, hyperphosphatemia, hyperpigmentation, hypertriglyceridemia, hyperuricemia, hypoalbuminemia, hypocalcemia, hypochloremia, hypoglycemia, hypokalemia, hypomagnesemia, hyponatremia, hypophosphatemia, impotence, infection, injection site reactions, insomnia, iron deficiency, itching, joint pain, kidney failure, leukopenia, liver dysfunction, memory loss, menopause, mouth sores, mucositis, muscle pain, myalgias, myelosuppression, myocarditis, neutropenic fever, nausea, nephrotoxicity, neutropenia, nosebleeds, numbness, ototoxicity, pain, palmar-plantar erythrodysesthesia, pancytopenia, pericarditis, peripheral neuropathy, pharyngitis, photophobia, photosensitivity, pneumonia, pneumonitis, proteinuria, pulmonary embolus, pulmonary fibrosis, pulmonary toxicity, rash, rapid heart beat, rectal bleeding, restlessness, rhinitis, seizures, shortness of breath, sinusitis, thrombocytopenia, tinnitus, urinary tract infection, vaginal bleeding, vaginal dryness, vertigo, water retention, weakness, weight loss, weight gain, and xerostomia. In general, toxicity is acceptable if the benefits to the subject achieved through the therapy outweigh the adverse events experienced by the subject due to the therapy.

### Neuroinflammatory Disorders

The methods described herein can be used to treat any subject in need thereof. As used herein, a “subject in need thereof” includes any subject that has a disease or disorder associated with a pathological neuroinflammatory response (e.g., an autoimmune disease, an immune disorder, an inflammatory disease, a neurodegenerative disease, a neuromuscular disease, a psychiatric disease), as well as any subject with an increased likelihood of acquiring a such a disease or disorder.

The compositions described herein can be used, for example, as a pharmaceutical composition for preventing or treating (reducing, partially or completely, the adverse effects of) neuroinflammation and/or a neuroinflammatory disease including, but not limited to, an autoimmune disease, an immune disorder, an inflammatory disease, a neurodegenerative disease, a neuromuscular disease, a psychiatric disease; a pharmaceutical composition for suppressing rejection in organ transplantation or other situations in which tissue rejection might occur; a supplement, food, or beverage for improving immune functions; or a reagent for modulating the proliferation or function of immune cells.

In some embodiments, the methods provided herein are useful for the treatment of neuroinflammation. In certain embodiments, the inflammation of nervous system, including but not limited to brain inflammation, peripheral nerves inflammation, neural inflammation, spinal cord inflammation, ocular inflammation, and/or other inflammation, as discussed below.

Examples of diseases associated with neuroinflammation which may be treated with the methods and compositions described herein include, but are not limited to, encephalitis (inflammation of the brain), encephalomyelitis (inflammation of the brain and spinal cord), meningitis (inflammation of the membranes that surround the brain and spinal cord), Guillain-Barre syndrome, neuromyotonia, narcolepsy, multiple sclerosis, myelitis, schizophrenia, acute disseminated encephalomyelitis (ADEM), accute optic neuritis (AON), transverse myelitis, neuromyelitis optica (NMO), Alzheimer’s disease, Parkinson’s disease, amyotrophic lateral sclerosis, frontotemporal lobar dementia, optic neuritis, neuromyelitis optica spectrum disorder (NMOSD), autoimmune encephalitis, anti-NMDA receptor encephalitis, Rasmussen’s encephalitis, acute necrotizing encephalopathy of childhood (ANEC), opsoclonus-myoclonus ataxia syndrome, traumatic brain injury, Huntington’s disease, depression, anxiety, migraine, myasthenia gravis, acute ischemic stroke, epilepsy, synucleinopathies, frontotemporal dementia, progressive nonfluent aphasia, semantic dementia, Nodding syndrome, cerebral ischemia, neuropathic pain, autism spectrum disorder, fibromyalgia syndrome, progressive supranuclear palsy, corticobasal degeneration, systemic lupus erythematosus, prion disease, motor neuron diseases (MND), spinocerebellar ataxia, spinal muscular atrophy, dystonia, idiopathicintracranial hypertension, nervous system disease, central nervous system disease, peripheral nervous system disease, movement disorders, encephalopathy, peripheral neuropathy, or post-operative cognitive dysfunction.

Examples of nervous system immune disorders which may be treated with the methods and compositions described herein include, but are not limited to, encephalitis, Guillain-Barre syndrome, meningitis, neuromyotonia, narcolepsy, multiple sclerosis, myelitis and schizophrenia.

The methods and compositions described herein may be used to treat diseases associated with activation of T helper 17 cells (Th17). Such conditions include, but are not limited to, multiple sclerosis, systemic lupus erythematosus, and encephalomyelitis.

### Methods of Making Enhanced Bacteria

In certain aspects, provided herein are methods of making engineered bacteria for the production of the mEVs (such as smEVs and/or pmEVs), bacteria for pharmaceutical compositions, or any combination thereof, described herein. In some embodiments, the engineered bacteria are modified to enhance certain desirable properties. For example, in some embodiments, the engineered bacteria are modified to enhance the immunomodulatory and/or therapeutic effect of the mEVs (such as smEVs and/or pmEVs), bacteria for pharmaceutical compositions, or any combination thereof, (e.g., either alone or in combination with another therapeutic agent), to reduce toxicity and/or to improve bacterial and/or mEV (such as smEV and/or pmEV) manufacturing (e.g., higher oxygen tolerance, improved freeze-thaw tolerance, shorter generation times). The engineered bacteria may be produced using any technique known in the art, including but not limited to site-directed mutagenesis, transposon mutagenesis, knock-outs, knock-ins, polymerase chain reaction mutagenesis, chemical mutagenesis, ultraviolet light mutagenesis, transformation (chemically or by electroporation), phage transduction, directed evolution, CRISPR/Cas9, or any combination thereof.

In some embodiments of the methods provided herein, the bacterium is modified by directed evolution. In some embodiments, the directed evolution comprises exposure of the bacterium to an environmental condition and selection of bacterium with improved survival and/or growth under the environmental condition. In some embodiments, the method comprises a screen of mutagenized bacteria using an assay that identifies enhanced bacterium. In some embodiments, the method further comprises mutagenizing the bacteria (e.g., by exposure to chemical mutagens and/or UV radiation) or exposing them to a therapeutic agent (e.g., antibiotic) followed by an assay to detect bacteria having the desired phenotype (e.g., an in vivo assay, an ex vivo assay, or an in vitro assay).

### EXAMPLES

### Example 1: Veillonella Parvula Strain A in SJL Relapsing-remitting Model Of Neuroinflammation

The SJL EAE model is a Th17/Th1-mediated mouse model of relapsing-remitting neuroinflammatory disease that is induced via immunization with proteolipid protein (PLP), a major protein constituent of CNS myelin. Disease pathology is due to infiltration of immune cells in the CNS leading to decreased motor function and paralysis. Briefly, in this model mice are injected subcutaneously with an emulsion of PLP-peptide plus CFA and intraperitoneally with pertussis toxin on day 0. Acute neuroinflammatory disease develops between day 10 to day 16, and the relapsing-remitting disease phase occurs from day 20 to day 45. Mice are dosed daily from the day of sensitization through the end of the study by oral gavage. In a therapeutic model, dosing of the mice begins on the second day of disease (~ Day 11 after disease induction). Mice are scored daily for a decrease in motor function and complete paralysis in the tail and limbs. Histopathology is carried out to score the frequency of inflammatory infiltrates and demyelination in the spinal cord.

### SJL EAE Mouse Model Details

Relapsing, remitting disease
EAE induced with subcutaneous immunization of PLP139-151 peptide/CFA emulsion followed by IP injection with pertussis toxin on day 0
Th17/Th1 mediated with strong Th17 response in acute phase (day 10-day 17)
Prophylactic or therapeutic treatment model
50% relapse expected in vehicle control group w/ PO QD dosing
Relapse defined as 1 point increase in disease score
15 mice per group
Readouts:
   EAE disease scored daily (day 9-day 44) (EAE scoring criteria are shown in Table 3)
   Percent relapse calculation
   Average disease score in relapse period
   CNS histopathology

TABLE 3

EAE Scoring Criteria for Mice

| | |
|---|---|
| Score | Clinical observations |
| 0 | No obvious changes in motor functions of the mouse in comparison to non-immunized mice. |
| | When picked up by the tail, the tail has tension and is erect. Hind legs are usually spread apart. When the mouse is walking, there is no gait or head tilting. |
| 1 | Limp tail. |
| | When the mouse is picked up by its tail, the whole tail drapes over your finger, instead of being erect. |
| 2 | Limp tail and weakness of hind legs. |
| | When mouse is picked up by its tail, legs are not spread apart but held closer together. When the mouse is observed when walking, it has an obviously wobbly walk. |
| 3 | Limp tail and complete paralysis of hind legs (most common). |
| | OR |
| | Limp tail with paralysis of one front and one hind leg. |
| | OR |
| | ALL of: |
| | Severe head tilting, |
| | Walking only along the edges of the cage, |
| | Pushing against the cage wall, |
| | Spinning when picked up by the tail. |
| 4 | Limp tail, complete hind leg and partial front leg paralysis. |
| | Mouse is minimally moving around the cage but appears alert and feeding. Usually, euthanasia is recommended after the mouse scores 4 for 2 days. When the mouse is euthanized due to severe paralysis, a score of 5 is entered for that mouse for the remainder of the experiment. |
| 5 | Complete hind and complete front leg paralysis, no movement around the cage. |
| | OR |
| | Mouse is spontaneously rolling in the cage. |
| | OR |
| | Mouse is found dead due to paralysis. |

### Objectives

Test the efficacy of gamma irradiated (G.I.) Veillonella parvula Strain A (Strain A) in the relapsing-remitting EAE model induced with PLP139-151 peptide in SJL mice.
Assess the effects of G.I. Strain A on daily disease score and CNS inflammation.

### Treatment

Vehicle (anaerobic sucrose buffer) PO QD day 0 - day 42
Strain A 10^9 TCC/dose PO QD day 0 - day 42
Anti-IL-17A antibody IP Q2D day 7 - day 15
Fingolimod, 1 mg/kg PO QD day 0 - day 42

G.I. Strain A was prepared as a biomass and was gamma irradiated prior to use.

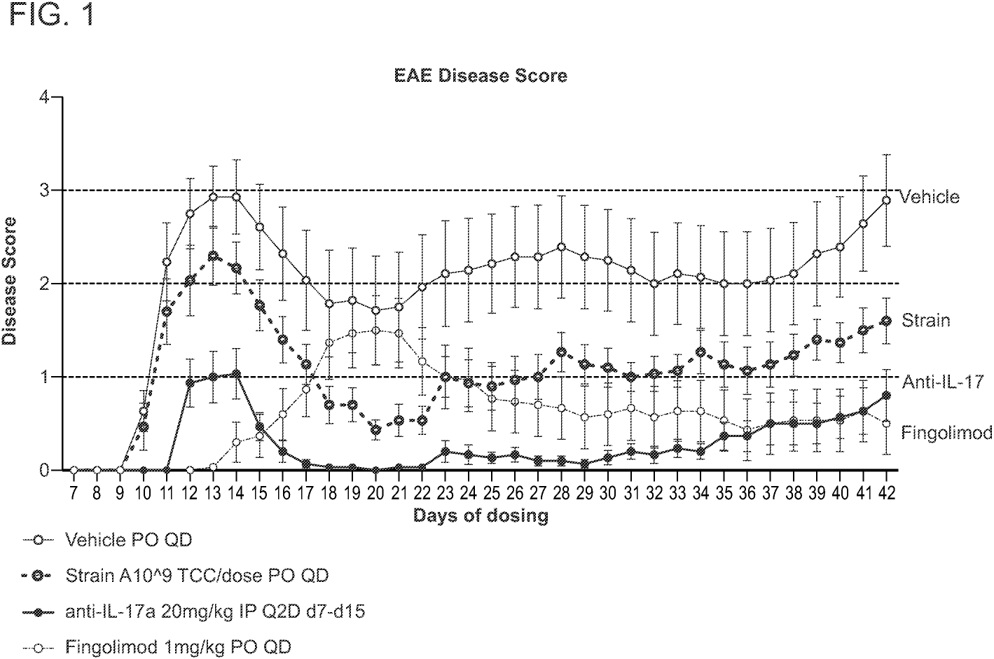

As shown in FIG. 1, G.I. Strain A reduced disease severity (as measured by disease score) in this relapse-remitting EAE model.

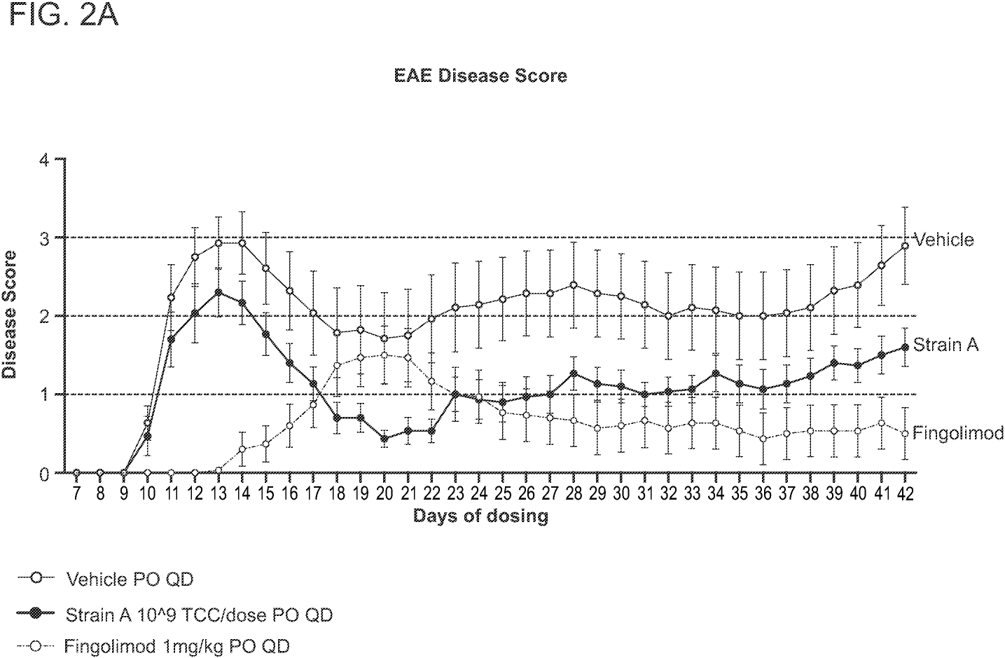

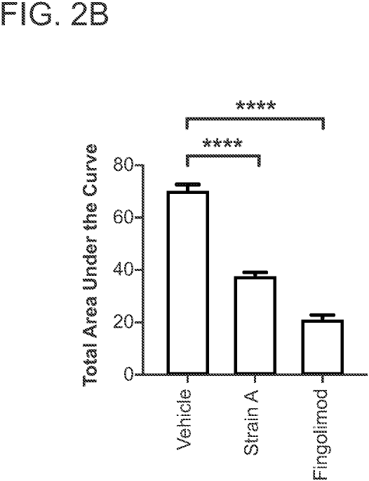

As shown in FIGS. 2A and 2B, G.I. Strain A reduced disease severity in the acute phase and relapsing-remitting phase as displayed by average daily disease score and total area under the curve.

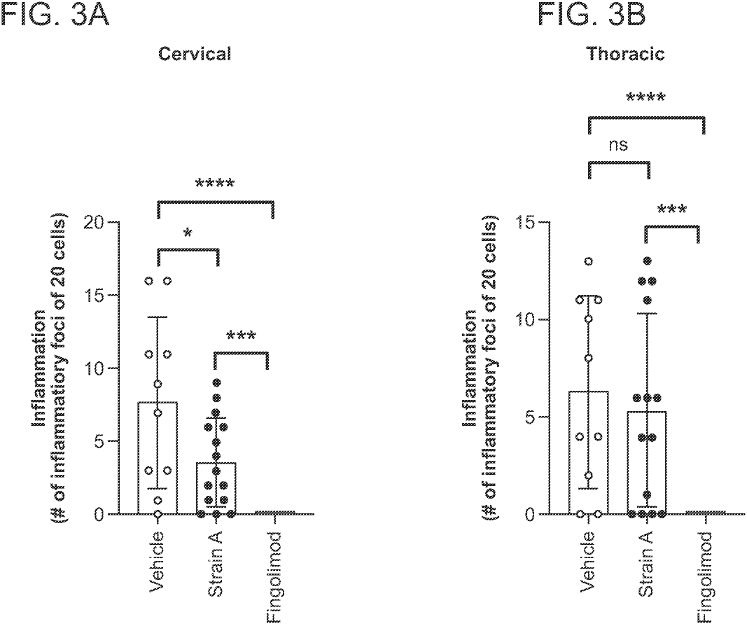

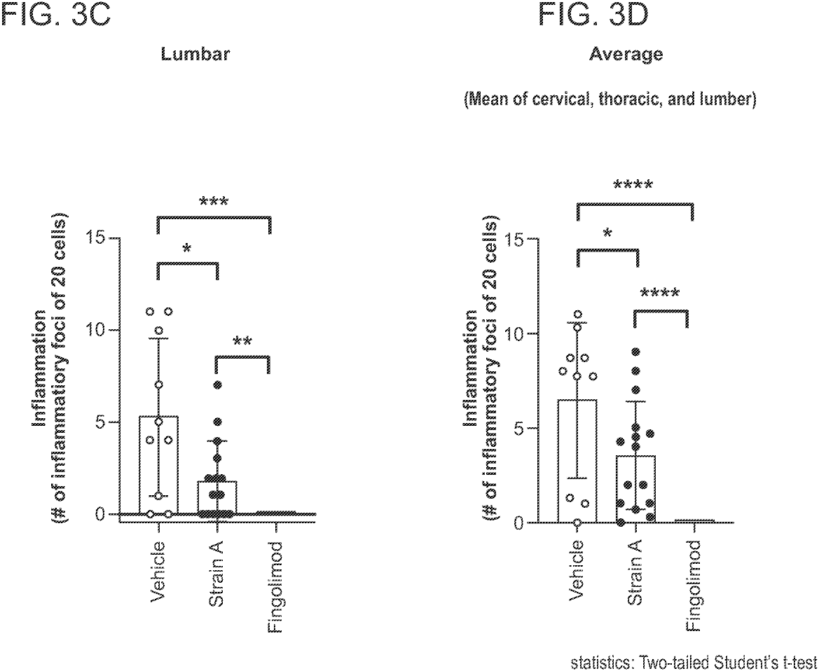

The results in FIGS. 3A-3D show that G.I. Strain A reduced inflammation of the spinal cord based on histopathological analysis of inflammatory foci in H&E stained tissue sections. FIG. 3A displays that inflammation was reduced in the cervical spinal cord region in mice treated with G.I. Strain A compared to mice treated with vehicle. FIG. 3B displays inflammation in the thoracic spinal cord where no significant difference in inflammation was observed. FIG. 3C displays that inflammation was reduced in the lumbar spinal cord region in mice treated with G.I. Strain A compared to vehicle. FIG. 3D displays average inflammation in the cervical, thoracic, and lumbar spinal cord regions. Dots represent individual mice. Statistical analysis was performed using a Student’s t-test.

Mean maximum score (MMS): A reduction in MMS means overall reduction in disease severity. G.I. Strain A treatment reduced relapse severity as shown by MMS of relapse period (data not shown).

### Conclusions

Strain A treatment given prophylactically reduced disease severity in the acute phase of EAE disease in SJL mice as shown by clinical score.
Strain A treatment reduced relapse severity as shown by clinical score and MMS of relapse period.
G.I. Strain A treatment reduced spinal cord inflammation as shown by histopathology scoring.

### Methods

Experimental Autoimmune Encephalomyelitis. Female SJL mice (8-10 weeks old) were subcutaneously injected at four sites with myelin proteolipid protein (PLP) 139-151 in CFA emulsion (0.05 mL/injection site; ~0.5 mg PLP PLP139-151/mL; Hooke Laboratories; EK-2120). Following immunization, EAE induction was completed by intraperitoneal injections of pertussis toxin (6 µg/mL; 0.1 mL/mouse) within 2 hours of immunization. Mice were randomized into groups and monitored for EAE clinical score over the course of 42 days. Disease progression was scored blinded of treatment or prior measurements. Disease severity was scored using standard EAE criteria: 0 (normal); 1 (loss of tail tone); 2 (hind limb weakness); 3 (hind limb paralysis); 4 (hind limb paralysis and forelimb paralysis or weakness); 5 (morbidity/death). Mice were observed daily for clinical symptoms. Mice were euthanized if they had a score of 4 for 2 days, and a score of 5 was recorded for remainder of the study for these animals.

End point tissue collection and histology. After euthanasia at the end of the study, EAE mice were perfused with 5-10 mL PBS and the spinal column was extracted from the base of the skull to the beginning of the pelvic bone. Spinal columns were then drop-fixed in 10% neutral buffered formalin and stored horizontally for 48 hours. After fixation, spinal columns were treated in mild formic acid decalcification solution (Immunocal-Statlab, Fisher Scientific, #141432) overnight (12-24 hours) at room temperature. Spinal columns were then trimmed into 4 mm-thick cervical, thoracic, and lumbar segments and processed using a Sakura Tissue Tek VIP 5 by graded alcohol dehydration, cleared in xylene, and finally infiltrated with paraffin. After processing, spinal column segments were embedded into paraffin blocks. Paraffin blocks were then sectioned at 4 µm on charged slides, air-dried overnight and stained with Hematoxylin and Eosin according to standard automated H&E protocol (Tissue-Tek Prisma) and then cover slipped (Tissue-Tek Glass). Prepared tissue sections were then imaged using a NanoZoomer 2.0 HT (Hamamatsu) at 20X magnification.

### Example 2: Therapeutic Treatment With Veillonella Parvula Strain A in SJL Relapsing-Remitting Model of Neuroinflammation

The SJL EAE model is a Th17/Th1-mediated mouse model of relapsing-remitting neuroinflammatory disease that is induced via immunization with proteolipid protein (PLP), a major protein constituent of CNS myelin. Disease pathology is due to infiltration of immune cells in the CNS leading to decreased motor function and paralysis. Briefly, in this model mice are injected subcutaneously with an emulsion of PLP-peptide plus CFA and intraperitoneally with pertussis toxin on day 0. Acute neuroinflammatory disease develops between day 10 to day 16, and the relapsing-remitting disease phase occurs from day 20 to day 45. Mice are dosed daily from the day of sensitization through the end of the study by oral gavage. In a therapeutic model, dosing of the mice begins on the second day of disease (~ Day 11 after disease induction). Mice are scored daily for a decrease in motor function and complete paralysis in the tail and limbs. Histopathology is carried out to score the frequency of inflammatory infiltrates and demyelination in the spinal cord.

Results were also generated demonstrating that therapeutic treatment with G.I. Strain A (e.g., PO QD dosing starting after the onset of disease) was also able to reduce disease severity in the relapsing-remitting phase of the SJL model of EAE as confirmed by disease score and total area under the curve.

The model details and read-outs, and methods are as described in Example 1, except that treatment began two days after disease onset, around day ~11 (mice were enrolled on the second day of disease for each mouse).

### Objectives

Test the efficacy of therapeutic treatment with Strain A in the relapse-remitting EAE model induced with PLP139-151 peptide in SJL mice.
Assess the effects of G.I. Strain A on daily disease score.

### Treatment

Vehicle (anaerobic sucrose buffer) PO QD day ~11 - day 42
G.I. Strain A 10 mg/dose PO QD day ~11 - day 42
Fingolimod, 1 mg/kg PO QD day ~11 - day 42

G.I. Strain A was prepared as a lyophilized powder and was gamma irradiated prior to use.

As shown in FIG. 4, therapeutic treatment with G.I. Strain A reduces EAE relapse severity, as measured by disease score.

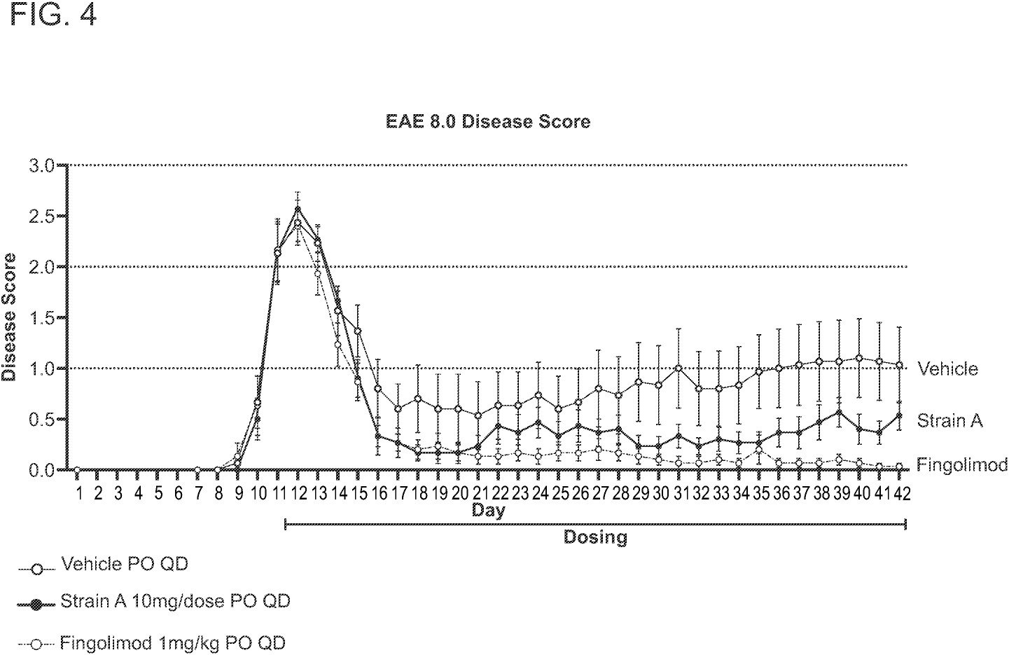

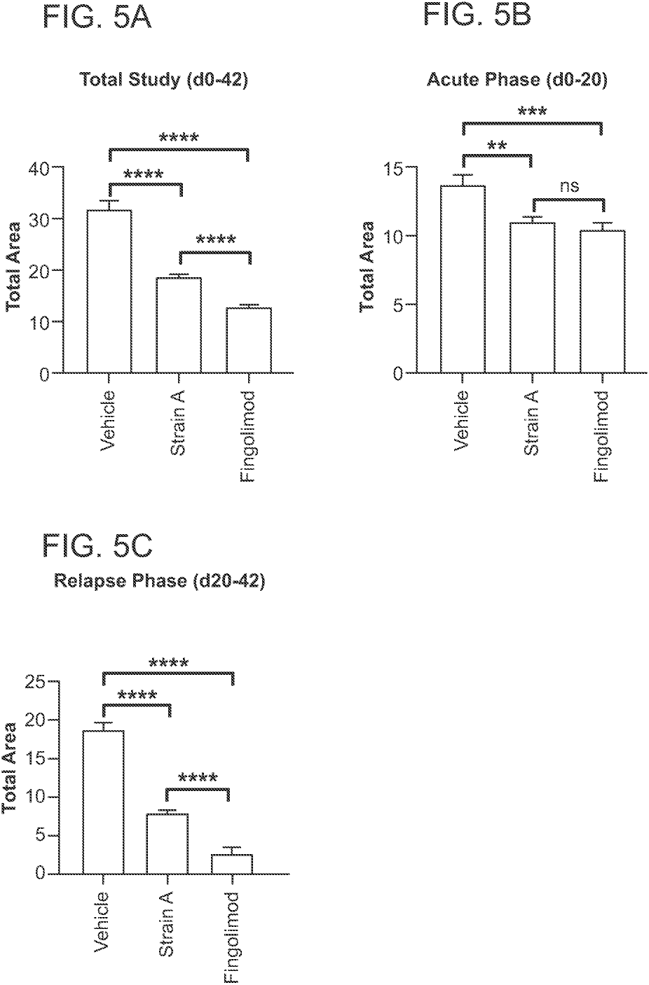

The results provided in FIGS. 5A- 5C show that therapeutic treatment with G.I. Strain A reduces AUC during relapsing-remitting phase of EAE. FIG. 5A displays the AUC for the total study (days 0-42). FIG. 5B displays the AUC for the acute phase (days 0-20). FIG. 5C displays the AUC for the relapse phase (days 20-42).

### Conclusions

Therapeutic oral treatment with G.I. Strain A reduced disease severity in the relapsing-remitting phase of the SJL model of EAE as confirmed by disease score.

### Example 3: Therapeutic Treatment With Veillonella Parvula Strain A in SJL Relapsing-Remitting Model of Neuroinflammation

The SJL EAE model is a Th17/Th1-mediated mouse model of relapsing-remitting neuroinflammatory disease that is induced via immunization with proteolipid protein (PLP), a major protein constituent of CNS myelin. Disease pathology is due to infiltration of immune cells in the CNS leading to decreased motor function and paralysis. Briefly, in this model mice are injected subcutaneously with an emulsion of PLP-peptide plus CFA and intraperitoneally with pertussis toxin on day 0. Acute neuroinflammatory disease develops between day 10 to day 16, and the relapsing-remitting disease phase occurs from day 20 to day 45. Mice are dosed daily from the day of sensitization through the end of the study by oral gavage. In a therapeutic model, dosing of the mice begins on the second day of disease (~ Day 11 after disease induction). Mice are scored daily for a decrease in motor function and complete paralysis in the tail and limbs. Histopathology is carried out to score the frequency of inflammatory infiltrates and demyelination in the spinal cord.

Results were also generated demonstrating that therapeutic treatment with G.I. Strain A (e.g., PO QD dosing starting after the onset of disease) was also able to reduce disease severity in the relapsing-remitting phase of the SJL model of EAE as confirmed by disease score and total area under the curve.

The model details and read-outs, and methods are described below. Treatment began two days after disease onset, around day ~11 (mice were enrolled on the second day of disease for each mouse).

### Objectives

Test the efficacy of therapeutic treatment with G.I. Strain A in the relapse-remitting EAE model induced with PLP₁₃₉₋₁₅₁ peptide in SJL mice.

Assess the effects of G.I. Strain A on daily disease score and CNS inflammation.

Compare efficacy of G.I. Strain A with clinically-relevant dose of SOC (Fingolimod 0.1 mg/kg)

### Methods

Experimental Autoimmune Encephalomyelitis. Female SJL mice (8-10 weeks old) were subcutaneously injected at four sites with myelin proteolipid protein (PLP) 139-151 in CFA emulsion (0.05 mL/injection site; ~0.5 mg PLP PLP139-151/mL; Hooke Laboratories; EK-2120). Following immunization, EAE induction was completed by intraperitoneal injections of pertussis toxin (6 µg/mL; 0.1 mL/mouse) within 2 hours of immunization. Mice were randomized into groups and monitored for EAE clinical score over the course of 42 days. Disease progression was scored blinded of treatment or prior measurements. Disease severity was scored using standard EAE criteria: 0 (normal); 1 (loss of tail tone); 2 (hind limb weakness); 3 (hind limb paralysis); 4 (hind limb paralysis and forelimb paralysis or weakness); 5 (morbidity/death). Mice were observed daily for clinical symptoms. Mice were euthanized if they had a score of 4 for 2 days, and a score of 5 was recorded for remainder of the study for these animals.

End point tissue collection and histology. After euthanasia at the end of the study, EAE mice were perfused with 5-10 mL PBS and the spinal column was extracted from the base of the skull to the beginning of the pelvic bone. Spinal columns were then drop-fixed in 10% neutral buffered formalin and stored horizontally for 48 hours. After fixation, spinal columns were treated in mild formic acid decalcification solution (Immunocal-Statlab, Fisher Scientific, #141432) overnight (12-24 hours) at room temperature. Spinal columns were then trimmed into 4 mm-thick cervical, thoracic, and lumbar segments and processed using a Sakura Tissue Tek VIP 5 by graded alcohol dehydration, cleared in xylene, and finally infiltrated with paraffin. After processing, spinal column segments were embedded into paraffin blocks. Paraffin blocks were then sectioned at 4 µm on charged slides, air-dried overnight and stained with Hematoxylin and Eosin according to standard automated H&E protocol (Tissue-Tek Prisma) and then cover slipped (Tissue-Tek Glass). Prepared tissue sections were then imaged using a NanoZoomer 2.0 HT (Hamamatsu) at 20X magnification.

### Treatment

Vehicle (anaerobic sucrose buffer) PO QD day ~11 - day 42
G.I. Strain A 10 mg/dose PO QD day ~11 - day 42
Fingolimod, 1 mg/kg PO QD day ~11 - day 42
Fingolimod, 0.1 mg/kg PO QD day ~11 - day 42

G.I. Strain A was prepared as a lyophilized powder and was gamma irradiated prior to use.

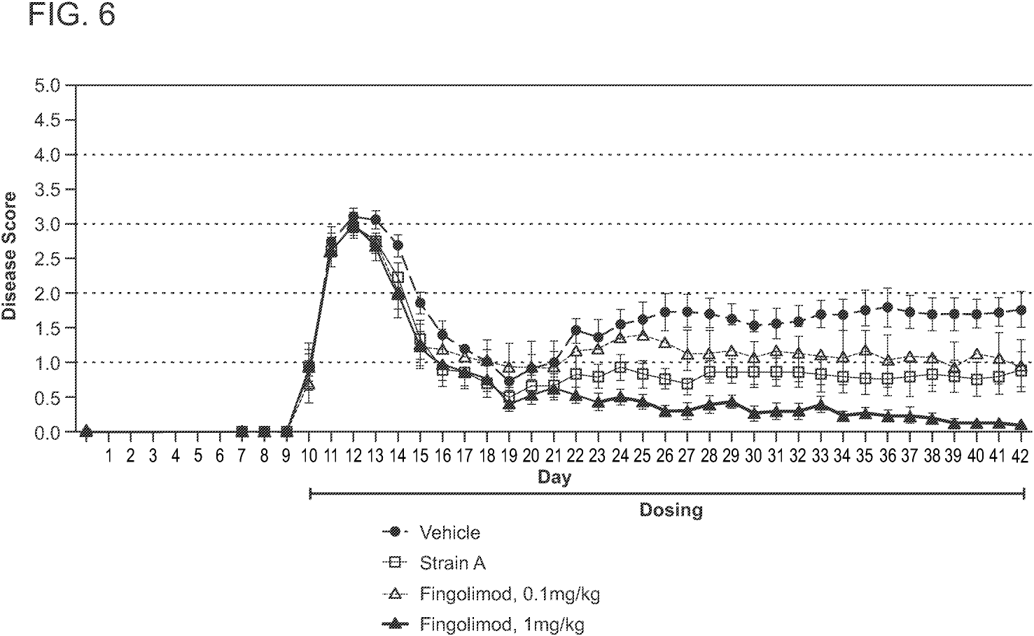

As shown in FIG. 6, therapeutic treatment with G.I. Strain A reduces EAE relapse severity, as measured by disease score.

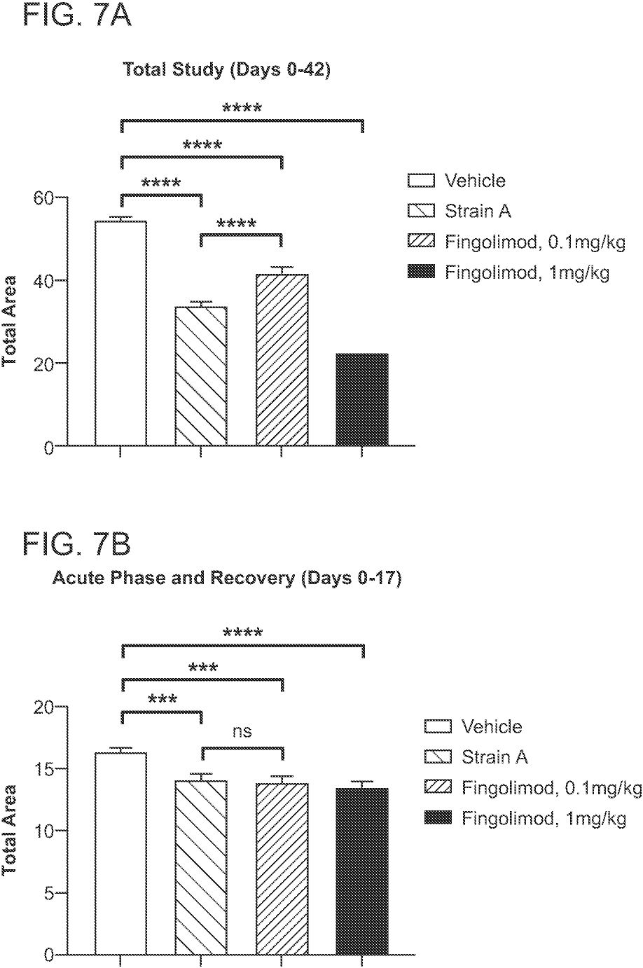

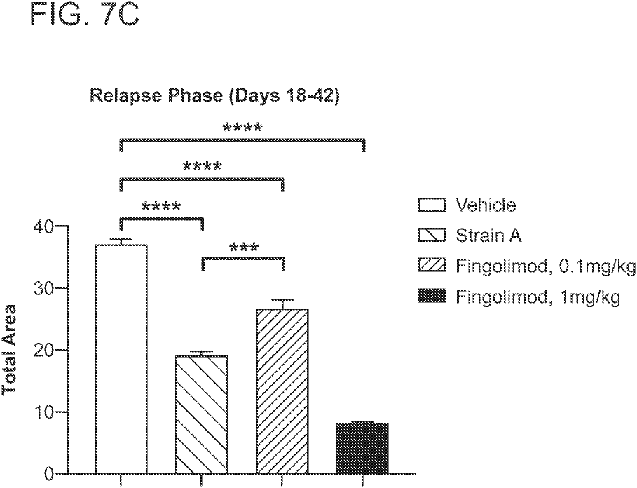

The results provided in FIGS. 7A- 7C show that therapeutic treatment with G.I. Strain A reduces AUC during relapsing-remitting phase of EAE. FIG. 7A displays the AUC for the total study (days 0-42). FIG. 7B displays the AUC for the acute phase (days 0-17). FIG. 7C displays the AUC for the relapse phase (days 18-42).

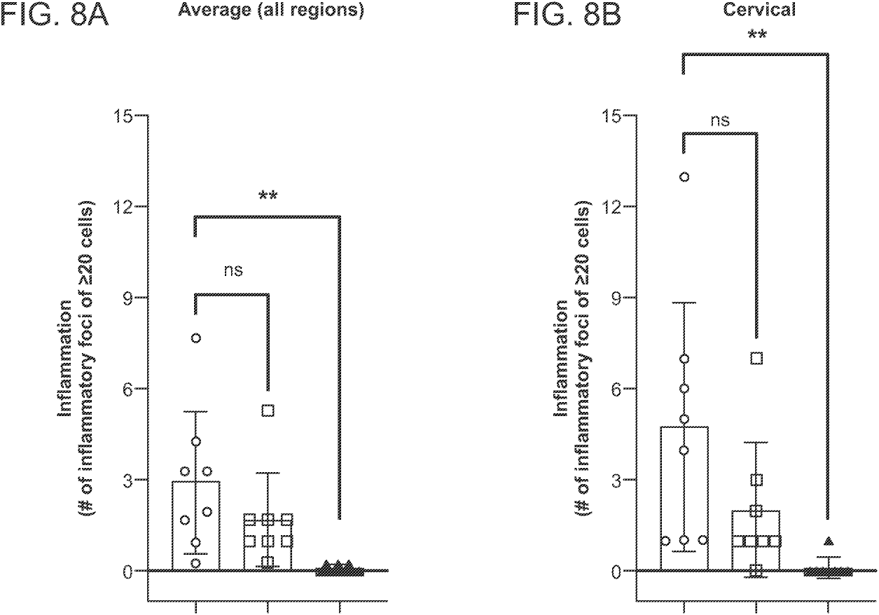

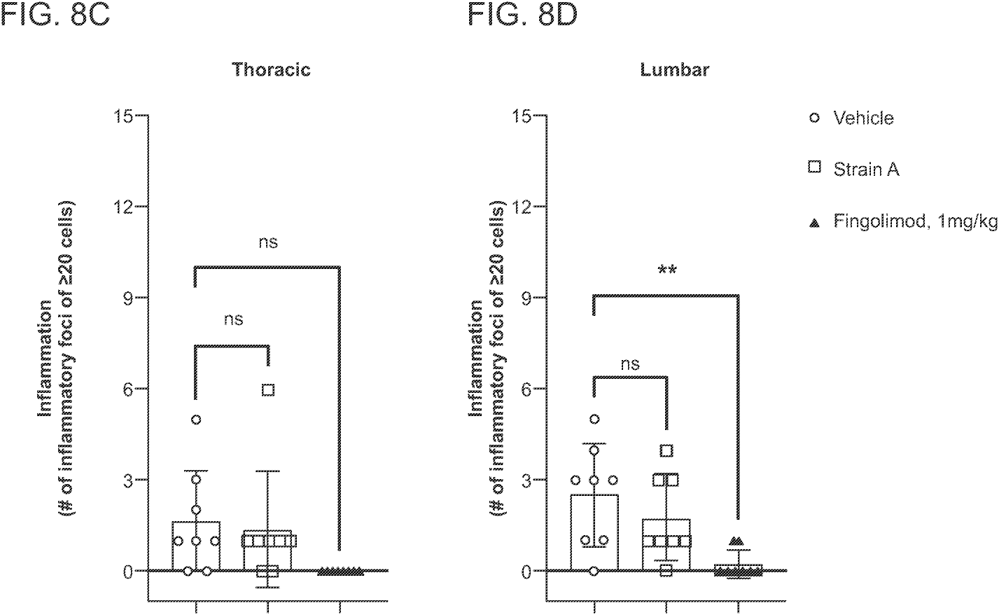

The results in FIGS. 8A-8D show that G.I. Strain A did not significantly reduce inflammation of the spinal cord based on histopathological analysis of inflammatory foci in H&E stained tissue sections. FIG. 8A displays average inflammation in the cervical, thoracic, and lumbar spinal cord regions. Dots represent individual mice. Bars show mean ± SEM. Statistical analysis was performed using a one-way ANOVA with Dunnett’s multiple comparisons. FIG. 8B displays that inflammation was reduced, although not to a level reaching statistical significance, in the cervical spinal cord region in mice treated with G.I. Strain A compared to mice treated with vehicle. FIG. 8C displays reduced inflammation, although not to a level reaching statistical significance, in the thoracic spinal cord. FIG. 8D displays that inflammation was reduced, although not to a level reaching statistical significance, in the lumbar spinal cord region in mice treated with G.I. Strain A compared to vehicle.

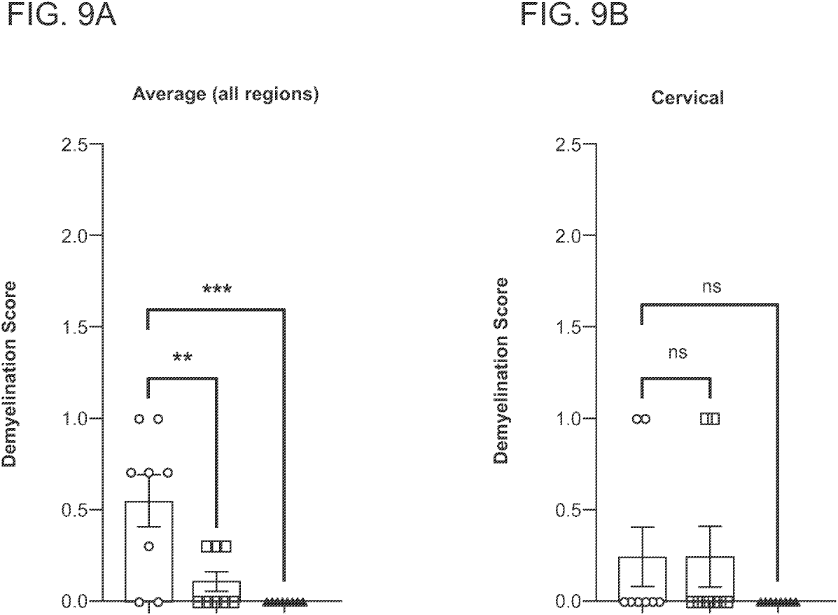

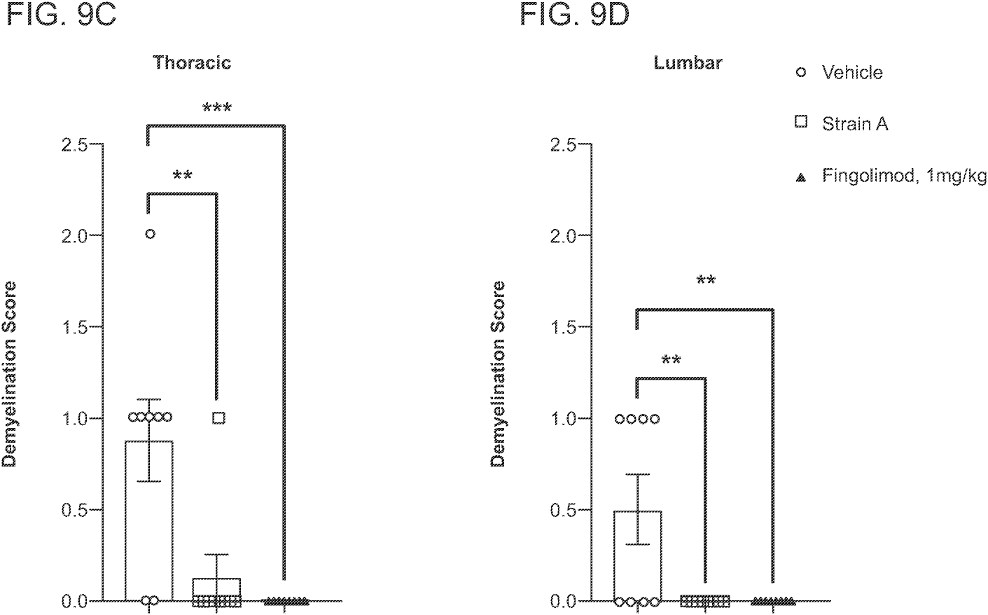

The results in FIGS. 9A-9D show that treatment with G.I. Strain A reduces demyelination of the spinal cord based on demyelination scores of spinal cord tissue sections. In these figures, dots represent individual mice. Bars show mean ± SEM. Statistical analysis was performed using a one-way ANOVA with Dunnett’s multiple comparisons. FIG. 9A shows a reduced average demyelination score in the cervical, thoracic, and lumbar spinal cord regions from subjects treated with G.I. Strain A relative to vehicle control. Table 4 shows the p-values for these averaged scores of test treatment versus vehicle.

TABLE 4

Average demyelination scores

| Treatment | Demyelination | | | | |
|---|---|---|---|---|---|
| | Cervical | Thoracic | Lumbar | Average | p value (Average) |
| Vehicle | 0.3±0.5 | 0.9±0.6 | 0.5±0.5 | 0.5±0.4 | - |
| Strain A | 0.3±0.5 | 0.1±0.4 | 0.0±0.0 | 0.1±0.2 | 0.0305 |
| Fingolimod, 1 mg/kg | 0.0±0.0 | 0.0±0.0 | 0.0±0.0 | 0.0±0.0 | 0.0036 |

FIG. 9B shows a non-significant change in the demyelination score in the cervical region in mice treated with G.I. Strain A relative to vehicle control. FIGS. 9C and 9D show significant reduction in demyelination scores in the thoracic and lumbar regions, respectively, of the mice treated with G.I. Strain A relative to vehicle control.

Mean maximum score (MMS): A reduction in MMS means overall reduction in disease severity. G.I. Strain A treatment reduced relapse severity as shown by MMS of relapse period (Tables 5 and 6).

TABLE 5

Clinical Results (Day 0-42)

| Treatment | Mean day of onset ±SD | Score at enrollment ± SD | MMS ± SD | p value |
|---|---|---|---|---|
| Vehicle | 10.4±0.5 | 3.03±0.48 | 3.37±0.35 | - |
| Strain A | 10.5±0.5 | 2.90±0.48 | 3.13±0.55 | 0.2303 |
| Fingolimod, 0.1 mg/kg | 10.5 | 3.00±0.46 | 3.20±0.70 | 0.2395 |
| Fingolimod, 1 mg/kg | 10.5±0.7 | 2.77±0.53 | 3.13±0.52 | 0.1647 |

| Treatment | End score ± SD | P value | End % body weight ± SD | p value |
|---|---|---|---|---|
| Vehicle | 1.77±1.02 | - | 93.4±7.9% | - |
| Strain A | 0.90±0.99 | 0.0284 | 96.0±8.0% | 0.3872 |
| Fingolimod, 0.1 mg/kg | 0.97±1.45 | 0.0285 | 95.2±11.1% | 0.6215 |
| Fingolimod, 1 mg/kg | 0.10±0.21 | <0.0001 | 101.8±12.0% | 0.0310 |

MMS compared using Wilcoxon’s non-parametric test
End EAE scores compared using Wilcoxon’s non-parametric test
Change in body weight at the end of study compared using two-tailed Student’s t-test
P values vs. vehicle

TABLE 6

Clinical Results (Acute Phase and Relapses)

| Treatment | MMS until Day 17 ± SD | p value | Incidence of relapse (%) | p value |
|---|---|---|---|---|
| Vehicle | 3.27±0.42 | - | 73.3% | - |
| Strain A | 3.13±0.55 | 0.5605 | 46.7% | 0.1331 |
| Fingolimod, 0.1 mg/kg | 3.13+0.69 | 0.2808 | 28.6% | 0.0142 |
| Fingolimod, 1 mg/kg | 3.13±0.52 | 0.4767 | 0% | <0.0001 |

| Treatment | MMS of relapse period ± SD (Days 18-42) | p value |
|---|---|---|
| Vehicle | 2.57±0.86 | - |
| Strain A | 1.60±0.86 | 0.0069 |
| Fingolimod, 0.1 mg/kg | 1.77±1.53 | 0.0814 |
| Fingolimod, 1 mg/kg | 0.83±0.67 | <0.0001 |

Disease incidence compared using chi-square test
Disease relapse compared using chi-square test
MMS compared using Wilcoxon’s non-parametric test
P vales vs vehicle

### Conclusions

Therapeutic oral dosing with G.I. Strain A reduced disease relapse severity in the SJL model of EAE as confirmed by disease score.
Therapeutic oral dosing with G.I. Strain A reduced demyelination of the spinal cord
Therapeutic oral dosing with G.I. Strain A was more efficacious at reducing disease relapse severity and demyelination than a clinically-relevant dose of Fingolimod
Therapeutic dosing of G.I. Strain A activated multiple gene sets in both lymphoid and myeloid immune cells
Therapeutic dosing of G.I. Strain A decreased axonal guidance signaling in the small intestine

### Example 4: Veillonella Parvula Strain A smEV Isolation and Enumeration

Equipment required:
Sorvall RC-5C centrifuge with SLA-3000 rotor
Optima XE-90 Ultracentrifuge by Beckman-Coulter 45Ti rotor
Sorvall wX+ Ultra Series Centrifuge by Thermo Scientific Fiberlite F37L-8×100 rotor

### 1. Microbial Supernatant Collection and Filtration

Microbes must be pelleted and filtered away from supernatant in order to recover smEVs and not microbes.
a. Pellet Microbial culture
   i. Use Sorvall RC-5C centrifuge with the SLA-3000 rotor and centrifuge culture for a minimum of 15 min at a minimum of 7,000 rpm.
   ii. Decant supernatant into new and sterile container.
b. Supernatant Filtration
   i. Filter supernatant through 0.2um filter.
   ii. For supernatants with poor filterability (less than 300 ml of supernatant pass through filter) attach a 0.45 um capsule filter ahead of the 0.2 um vacuum filter.
   iii. Store ‘filtered’ supernatant at 4° C.
   iv. Filtered supernatant can then be concentrated using TFF.

### 2. Isolation of smEVs Using Ultracentrifugation

Centrifuging concentrated supernatant in the ultracentrifuge will pellet smEVs isolating the smEVs from smaller biomolecules.
i. Set speed for 200,000 g, time for 1 hour, and temperature at 4° C.
ii. When rotor has stopped, remove tubes from ultracentrifuge and gently pour off the supernatant.
iii. Add more supernatant, balance, and centrifuge tubes again.
iv. After all concentrated supernatant has been centrifuged, the pellets generated are referred to as ‘crude’ smEV pellets.
v. Add sterile 1xPBS to pellets and place in container. Place on shaker, speed 70, in 4° C. fridge overnight or longer.
vi. Resuspend the smEV pellets with additional sterile 1xPBS.
   1. Store resuspended crude smEV samples at 4° C. or at -80° C.

### 3. smEV Purification Using Density Gradients

Density gradients are used for smEV purification. During ultracentrifugation, particles in the sample will move, and separate, within the graded density medium based on their ‘buoyant’ densities. In this way smEVs are separated from other particles, such as sugars, lipids, or other proteins, in the sample.
a. Preparation of Density Medium
   i. For smEV purification, four different percentages of the density medium (60% Optiprep) are used, a 45% layer, a 35% layer, a 25%, and a 15% layer. This will create the graded layers. A 0% layer is added at the top consisting of sterile 1xPBS.
   ii. The 45% gradient layer should contain the crude smEV sample. 5ml of sample is added to 15 ml of Optiprep. If crude smEV sample is less than 5 ml, bring up to volume using sterile 1xPBS.
b. Density Gradient Assembly
   i. Using a serological pipette, gently pipette the 45% gradient mixture up and down to mix. Then pipette the sample into a labeled clean and sterile ultracentrifuge tube.
   ii. Next, using a 10 ml serological pipette, slowly add 13 ml of 35% gradient mixture.
   iii. Continue with 13ml of the 25% gradient mixture followed by 13ml of the 15% mixture and finally 6 ml of sterile 1xPBS.
   iv. Balance ultracentrifuge tubes with sterile 1xPBS.
   v. Carefully place gradients in rotor and set the ultracentrifuge for 200,000 g and the temperature for 4° C. Centrifuge a minimum of 16 hours.
c. Removing Purified smEVs from Density Gradients
   i. Using a clean pipette, removed fraction(s) of interest and add to 15ml conical tube.
   ii. Keep ‘purified’ smEV samples at 4° C.
d. Removing Optiprep Material from Purified smEVs
   i. In order to clean and remove residual optiprep from smEVs, 10x volume of PBS should be added to purified smEVs.
   ii. Set the ultracentrifuge for 200,000 g and the temperature for 4° C. Centrifuge for 1 hour.
   iii. Carefully remove tubes from ultracentrifuge and decant supernatant.
   iv. Continue ‘washing’ purified smEVs until all sample has been pelleted.
   v. Add sterile 1xPBS to purified pellets and place in container. Place on shaker, speed 70, in 4° C. fridge overnight or longer.
   vi. Resuspend the ‘purified’ smEV pellets with additional sterile 1xPBS.
      1. Store resuspended purified smEV samples at 4° C. or at -80° C. .

### Example 5: Labeling Bacterial pmEVs

pmEVs may be labeled in order to track their biodistribution in vivo and to quantify and track cellular localization in various preparations and in assays conducted with mammalian cells. For example, pmEVs may be radio-labeled, incubated with dyes, fluorescently labeled, luminescently labeled, or labeled with conjugates containing metals and isotopes of metals.

For example, pmEVs may be incubated with dyes conjugated to functional groups such as NHS-ester, click-chemistry groups, streptavidin or biotin. The labeling reaction may occur at a variety of temperatures for minutes or hours, and with or without agitation or rotation. The reaction may then be stopped by adding a reagent such as bovine serum albumin (BSA), or similar agent, depending on the protocol, and free or unbound dye molecule removed by ultra-centrifugation, filtration, centrifugal filtration, column affinity purification or dialysis. Additional washing steps involving wash buffers and vortexing or agitation may be employed to ensure complete removal of free dyes molecules such as described in Su Chul Jang et al, Small. 11, No.4, 456-461(2017).

Optionally, pmEVs may be concentrated to 5.0 E12 particle/ml (300ug) and diluted up to 1.8mo using 2X concentrated PBS buffer pH 8.2 and pelleted by centrifugation at 165,000 × g at 4 C using a benchtop ultracentrifuge. The pellet is resuspended in 300 ul 2X PBS pH 8.2 and an NHS-ester fluorescent dye is added at a final concentration of 0.2 mM from a 10 mM dye stock (dissolved in DMSO). The sample is gently agitated at 24° C. for 1.5 hours, and then incubated overnight at 4° C. Free non-reacted dye is removed by 2 repeated steps of dilution/pelleting as described above, using 1X PBS buffer, and resuspending in 300ul final volume.

Fluorescently labeled pmEVs are detected in cells or organs, or in in vitro and/or ex vivo samples by confocal microscopy, nanoparticle tracking analysis, flow cytometry, fluorescence activated cell sorting (FACs) or fluorescent imaging system such as the Odyssey CLx LICOR (see e.g., Wiklander et al. 2015. J. Extracellular Vesicles. 4:10.3402/jev.v4.26316). Additionally, fluorescently labeled pmEVs are detected in whole animals and/or dissected organs and tissues using an instrument such as the IVIS spectrum CT (Perkin Elmer) or Pearl Imager, as in H-I. Choi, et al. Experimental & Molecular Medicine. 49: e330 (2017).

pmEVs may be labeled with conjugates containing metals and isotopes of metals using the protocols described above. Metal-conjugated pmEVs may be administered in vivo to animals. Cells may then be harvested from organs at various time-points, and analyzed ex vivo. Alternatively, cells derived from animals, humans, or immortalized cell lines may be treated with metal-labelled pmEVs in vitro and cells subsequently labelled with metal-conjugated antibodies and phenotyped using a Cytometry by Time of Flight (CyTOF) instrument such as the Helios CyTOF (Fluidigm) or imaged and analyzed using and Imaging Mass Cytometry instrument such as the Hyperion Imaging System (Fluidigm). Additionally, pmEVs may be labelled with a radioisotope to track the pmEVs biodistribution (see, e.g., Miller et al., Nanoscale. 2014 May 7;6(9):4928-35).

### Example 6: Transmission Electron Microscopy to Visualize Bacterial pmEVs

pmEVs are prepared from bacteria batch cultures. Transmission electron microscopy (TEM) may be used to visualize purified bacterial pmEVs (S. Bin Park, et al. PLoS ONE. 6(3):e17629 (2011). pmEVs are mounted onto 300- or 400-mesh-size carbon-coated copper grids (Electron Microscopy Sciences, USA) for 2 minutes and washed with deionized water. pmEVs are negatively stained using 2% (w/v) uranyl acetate for 20 sec - 1 min. Copper grids are washed with sterile water and dried. Images are acquired using a transmission electron microscope with 100-120 kV acceleration voltage. Stained pmEVs appear between 20-600 nm in diameter and are electron dense. 10-50 fields on each grid are screened.

### Example 7: Profiling pmEV Composition and Content

pmEVs may be characterized by any one of various methods including, but not limited to, NanoSight characterization, SDS-PAGE gel electrophoresis, Western blot, ELISA, liquid chromatography-mass spectrometry and mass spectrometry, dynamic light scattering, lipid levels, total protein, lipid to protein ratios, nucleic acid analysis and/or zeta potential.

### NanoSight Characterization of pmEVs

Nanoparticle tracking analysis (NTA) is used to characterize the size distribution of purified bacterial pmEVs. Purified pmEV preparations are run on a NanoSight machine (Malvern Instruments) to assess pmEV size and concentration.

### SDS-PAGE Gel Electrophoresis

To identify the protein components of purified pmEVs, samples are run on a gel, for example a Bolt Bis-Tris Plus 4-12% gel (Thermo-Fisher Scientific), using standard techniques. Samples are boiled in 1x SDS sample buffer for 10 minutes, cooled to 4° C., and then centrifuged at 16,000 × g for 1 min. Samples are then run on a SDS-PAGE gel and stained using one of several standard techniques (e.g., Silver staining, Coomassie Blue, Gel Code Blue) for visualization of bands.

### Western Blot Analysis

To identify and quantify specific protein components of purified pmEVs, pmEV proteins are separated by SDS-PAGE as described above and subjected to Western blot analysis (Cvjetkovic et al., Sci. Rep. 6, 36338 (2016)) and are quantified via ELISA. pmEV proteomics and Liquid Chromatography-Mass Spectrometry (LC-MS/MS) and Mass Spectrometry (MS)

Proteins present in pmEVs are identified and quantified by Mass Spectrometry techniques. pmEV proteins may be prepared for LC-MS/MS using standard techniques including protein reduction using dithiotreitol solution (DTT) and protein digestion using enzymes such as LysC and trypsin as described in Erickson et al, 2017 (Molecular Cell, VOLUME 65, ISSUE 2, P361-370, JANUARY 19, 2017). Alternatively, peptides are prepared as described by Liu et al. 2010 (JOURNAL OF BACTERIOLOGY, June 2010, p. 2852-2860 Vol. 192, No. 11), Kieselbach and Oscarsson 2017 (Data Brief. 2017 Feb; 10: 426-431.), Vildhede et al, 2018 (Drug Metabolism and Disposition Feb. 8, 2018). Following digestion, peptide preparations are run directly on liquid chromatography and mass spectrometry devices for protein identification within a single sample. For relative quantitation of proteins between samples, peptide digests from different samples are labeled with isobaric tags using the iTRAQ Reagent-8plex Multiplex Kit (Applied Biosystems, Foster City, CA) or TMT 10plex and 11plex Label Reagents (Thermo Fischer Scientific, San Jose, CA, USA). Each peptide digest is labeled with a different isobaric tag and then the labeled digests are combined into one sample mixture. The combined peptide mixture is analyzed by LC-MS/MS for both identification and quantification. A database search is performed using the LC-MS/MS data to identify the labeled peptides and the corresponding proteins. In the case of isobaric labeling, the fragmentation of the attached tag generates a low molecular mass reporter ion that is used to obtain a relative quantitation of the peptides and proteins present in each pmEV.

Additionally, metabolic content is ascertained using liquid chromatography techniques combined with mass spectrometry. A variety of techniques exist to determine metabolomic content of various samples and are known to one skilled in the art involving solvent extraction, chromatographic separation and a variety of ionization techniques coupled to mass determination (Roberts et al 2012 Targeted Metabolomics. Curr Protoc Mol Biol. 30: 1-24; Dettmer et al 2007, Mass spectrometry-based metabolomics. Mass Spectrom Rev. 26(1):51-78). As a non-limiting example, a LC-MS system includes a 4000 QTRAP triple quadrupole mass spectrometer (AB SCIEX) combined with 1100 Series pump (Agilent) and an HTS PAL autosampler (Leap Technologies). Media samples or other complex metabolic mixtures (~10 µL) are extracted using nine volumes of 74.9:24.9:0.2 (v/v/v) acetonitrile/methanol/formic acid containing stable isotope-labeled internal standards (valine-d8, Isotec; and phenylalanine-d8, Cambridge Isotope Laboratories). Standards may be adjusted or modified depending on the metabolites of interest. The samples are centrifuged (10 minutes, 9,000 g, 4° C.), and the supernatants (10 µL) are submitted to LCMS by injecting the solution onto the HILIC column (150 × 2.1 mm, 3 µm particle size). The column is eluted by flowing a 5% mobile phase [10mM ammonium formate, 0.1% formic acid in water] for 1 minute at a rate of 250uL/minute followed by a linear gradient over 10 minutes to a solution of 40% mobile phase [acetonitrile with 0.1% formic acid]. The ion spray voltage is set to 4.5 kV and the source temperature is 450° C.

The data are analyzed using commercially available software like Multiquant 1.2 from AB SCIEX for mass spectrum peak integration. Peaks of interest should be manually curated and compared to standards to confirm the identity of the peak. Quantitation with appropriate standards is performed to determine the number of metabolites present in the initial media, and after bacterial conditioning. A non-targeted metabolomics approach may also be used using metabolite databases, such as but not limited to the NIST database, for peak identification.

### Dynamic Light Scattering (DLS)

DLS measurements, including the distribution of particles of different sizes in different pmEV preparations are taken using instruments such as the DynaPro NanoStar (Wyatt Technology) and the Zetasizer Nano ZS (Malvern Instruments).

### Lipid Levels

Lipid levels are quantified using FM4-64 (Life Technologies), by methods similar to those described by A.J. McBroom et al. J Bacteriol 188:5385-5392. and A. Frias, et al. Microb Ecol. 59:476-486 (2010). Samples are incubated with FM4-64 (3.3 µg/mL in PBS for 10 minutes at 37° C. in the dark). After excitation at 515 nm, emission at 635 nm is measured using a Spectramax M5 plate reader (Molecular Devices). Absolute concentrations are determined by comparison of unknown samples to standards (such as palmitoyloleoylphosphatidylglycerol (POPG) vesicles) of known concentrations. Lipidomics can be used to identify the lipids present in the pmEVs.

### Total Protein

Protein levels are quantified by standard assays such as the Bradford and BCA assays. The Bradford assays are run using Quick Start Bradford 1x Dye Reagent (Bio-Rad), according to manufacturer’s protocols. BCA assays are run using the Pierce BCA Protein Assay Kit (Thermo-Fisher Scientific). Absolute concentrations are determined by comparison to a standard curve generated from BSA of known concentrations. Alternatively, protein concentration can be calculated using the Beer-Lambert equation using the sample absorbance at 280 nm (A280) as measured on a Nanodrop spectrophotometer (Thermo-Fisher Scientific).In addition, proteomics may be used to identify proteins in the sample.

### LipidProtein Ratios

Lipid:protein ratios are generated by dividing lipid concentrations by protein concentrations. These provide a measure of the purity of vesicles as compared to free protein in each preparation.

### Nucleic Acid Analysis

Nucleic acids are extracted from pmEVs and quantified using a Qubit fluorometer. Size distribution is assessed using a BioAnalyzer and the material is sequenced.

### Zeta Potential

The zeta potential of different preparations are measured using instruments such as the Zetasizer ZS (Malvern Instruments).

### Example 8: In Vitro Detection of pmEVs in Antigen-Presenting Cells

Dendritic cells in the lamina propria constantly sample live bacteria, dead bacteria, and microbial products in the gut lumen by extending their dendrites across the gut epithelium, which is one way that pmEVs produced by bacteria in the intestinal lumen may directly stimulate dendritic cells. The following methods represent a way to assess the differential uptake of pmEVs by antigen-presenting cells. Optionally, these methods may be applied to assess immunomodulatory behavior of pmEVs administered to a patient.

Dendritic cells (DCs) are isolated from human or mouse bone marrow, blood, or spleens according to standard methods or kit protocols (e.g., Inaba K, Swiggard WJ, Steinman RM, Romani N, Schuler G, 2001. Isolation of dendritic cells. Current Protocols in Immunology. Chapter 3:Unit3.7).

To evaluate pmEV entrance into and/or presence in DCs, 250,000 DCs are seeded on a round cover slip in complete RPMI-1640 medium and are then incubated with pmEVs from single bacterial strains or combinations pmEVs at various ratios. Purified pmEVs may be labeled with fluorochromes or fluorescent proteins. After incubation for various timepoints (e.g., 1 hour, 2 hours), the cells are washed twice with ice-cold PBS and detached from the plate using trypsin. Cells are either allowed to remain intact or are lysed. Samples are then processed for flow cytometry. Total internalized pmEVs are quantified from lysed samples, and percentage of cells that uptake pmEVs is measured by counting fluorescent cells. The methods described above may also be performed in substantially the same manner using macrophages or epithelial cell lines (obtained from the ATCC) in place of DCs.

### Example 9: Determining the Biodistribution of pmEVs When Delivered Orally to Mice

Wild-type mice (e.g., C57BL/6 or BALB/c) are orally inoculated with the pmEV composition of interest to determine the in vivo biodistibution profile of purified pmEVs. pmEVs are labeled to aide in downstream analyses. Alternatively, mice with some immune disorder (e.g., systemic lupus erythematosus, experimental autoimmune encephalomyelitis, NASH) may be studied for in vivo distribution of pmEVs over a given time-course.

Mice can receive a single dose of the pmEV (e.g., 25-100 µg) or several doses over a defined time course (25-100 µg). Alternatively, pmEVs dosages may be administered based on particle count (e.g., 7e+08 to 6e+11 particles). Mice are housed under specific pathogen-free conditions following approved protocols. Alternatively, mice may be bred and maintained under sterile, germ-free conditions. Blood, stool, and other tissue samples can be taken at appropriate time points.

The mice are humanely sacrificed at various time points (i.e., hours to days) post administration of the pmEV compositions, and a full necropsy under sterile conditions is performed. Following standard protocols, lymph nodes, adrenal glands, liver, colon, small intestine, cecum, stomach, spleen, kidneys, bladder, pancreas, heart, skin, lungs, brain, and other tissue of interest are harvested and are used directly or snap frozen for further testing. The tissue samples are dissected and homogenized to prepare single-cell suspensions following standard protocols known to one skilled in the art. The number of pmEVs present in the sample is then quantified through flow cytometry. Quantification may also proceed with use of fluorescence microscopy after appropriate processing of whole mouse tissue (Vankelecom H., Fixation and paraffin-embedding of mouse tissues for GFP visualization, Cold Spring Harb. Protoc., 2009). Alternatively, the animals may be analyzed using live-imaging according to the pmEV labeling technique.

Biodistribution may be performed in mouse models of autoimmunity such as but not limited to EAE and DTH (see, e.g., Turjeman et al., PLoS One 10(7): e0130442 (20105).

### Example 10: Purification and Preparation of Secreted Microbial Extracellular Vesicles (smEVs) from Bacteria

### Purification

Secreted microbial extracellular vesicles (smEVs) are purified and prepared from bacterial cultures using methods known to those skilled in the art (S. Bin Park, et al. PLoS ONE. 6(3):e17629 (2011)).

For example, bacterial cultures are centrifuged at 10,000-15,500 × g for 10-40 min at 4° C. or room temperature to pellet bacteria. Culture supernatants are then filtered to include material ≤ 0.22 µm (for example, via a 0.22 µm or 0.45 µm filter) and to exclude intact bacterial cells. Filtered supernatants are concentrated using methods that may include, but are not limited to, ammonium sulfate precipitation, ultracentrifugation, or filtration. Briefly, for ammonium sulfate precipitation, 1.5-3 M ammonium sulfate is added to filtered supernatant slowly, while stirring at 4° C. Precipitations are incubated at 4° C. for 8-48 hours and then centrifuged at 11,000 × g for 20-40 min at 4° C. The pellets contain smEVs and other debris. Briefly, using ultracentrifugation, filtered supernatants are centrifuged at 100,000-200,000 × g for 1-16 hours at 4° C. The pellet of this centrifugation contains smEVs and other debris. Briefly, using a filtration technique, using an Amicon Ultra spin filter or by tangential flow filtration, supernatants are filtered so as to retain species of molecular weight > 50, 100, 300, or 500 kDa.

Alternatively, smEVs are obtained from bacterial cultures continuously during growth, or at selected time points during growth, by connecting a bioreactor to an alternating tangential flow (ATF) system (e.g., XCell ATF from Repligen) according to manufacturer’s instructions. The ATF system retains intact cells (> 0.22 um) in the bioreactor, and allows smaller components (e.g., smEVs, free proteins) to pass through a filter for collection. For example, the system may be configured so that the < 0.22 um filtrate is then passed through a second filter of 100 kDa, allowing species such as smEVs between 0.22 um and 100 kDa to be collected, and species smaller than 100 kDa to be pumped back into the bioreactor. Alternatively, the system may be configured to allow for medium in the bioreactor to be replenished and/or modified during growth of the culture. smEVs collected by this method may be further purified and/or concentrated by ultracentrifugation or filtration as described above for filtered supernatants.

smEVs obtained by methods described above may be further purified by gradient ultracentrifugation, using methods that may include, but are not limited to, use of a sucrose gradient or Optiprep gradient. Briefly, using a sucrose gradient method, if ammonium sulfate precipitation or ultracentrifugation were used to concentrate the filtered supernatants, pellets are resuspended in 60% sucrose, 30 mM Tris, pH 8.0. If filtration was used to concentrate the filtered supernatant, the concentrate is buffer exchanged into 60% sucrose, 30 mM Tris, pH 8.0, using an Amicon Ultra column. Samples are applied to a 35-60% discontinuous sucrose gradient and centrifuged at 200,000 × g for 3-24 hours at 4° C. Briefly, using an Optiprep gradient method, if ammonium sulfate precipitation or ultracentrifugation were used to concentrate the filtered supernatants, pellets are resuspended in 45% Optiprep in PBS. If filtration was used to concentrate the filtered supernatant, the concentrate is diluted using 60% Optiprep to a final concentration of 45% Optiprep. Samples are applied to a 0-45% discontinuous sucrose gradient and centrifuged at 200,000 × g for 3-24 hours at 4° C. Alternatively, high resolution density gradient fractionation could be used to separate smEVs based on density.

### Preparation

To confirm sterility and isolation of the smEV preparations, smEVs are serially diluted onto agar medium used for routine culture of the bacteria being tested and incubated using routine conditions. Non-sterile preparations are passed through a 0.22 um filter to exclude intact cells. To further increase purity, isolated smEVs may be DNase or proteinase K treated.

Alternatively, for preparation of smEVs used for in vivo injections, purified smEVs are processed as described previously (G. Norheim, et al. PLoS ONE. 10(9): e0134353 (2015)). Briefly, after sucrose gradient centrifugation, bands containing smEVs are resuspended to a final concentration of 50 µg/mL in a solution containing 3% sucrose or other solution suitable for in vivo injection known to one skilled in the art. This solution may also contain adjuvant, for example aluminum hydroxide at a concentration of 0-0.5% (w/v).

To make samples compatible with further testing (e.g., to remove sucrose prior to TEM imaging or in vitro assays), samples are buffer exchanged into PBS or 30 mM Tris, pH 8.0 using filtration (e.g., Amicon Ultra columns), dialysis, or ultracentrifugation (following 15-fold or greater dilution in PBS, 200,000 × g, 1-3 hours, 4° C.) and resuspension in PBS.

For all of these studies, smEVs may be heated, irradiated, and/or lyophilized prior to administration as described above.

### Example 11: Manipulating Bacteria Through Stress to Produce Various Amounts of smEVs and/or to Vary Content of smEVs

Stress, and in particular envelope stress, has been shown to increase production of smEVs by some bacterial strains (I. MacDonald, M. Kuehn. J Bacteriol 195(13): doi: 10/1128/JB.02267-12). In order to vary production of smEVs by bacteria, bacteria are stressed using various methods.

Bacteria may be subjected to single stressors or stressors in combination. The effects of different stressors on different bacteria is determined empirically by varying the stress condition and determining the IC50 value (the conditions required to inhibit cell growth by 50%). smEV purification, quantification, and characterization occurs. smEV production is quantified (1) in complex samples of bacteria and smEVs by nanoparticle tracking analysis (NTA) or transmission electron microscopy (TEM); or (2) following smEV purification by NTA, lipid quantification, or protein quantification. smEV content is assessed following purification by methods described above.

### Antibiotic Stress

Bacteria are cultivated under standard growth conditions with the addition of sublethal concentrations of antibiotics. This may include 0.1-1 µg/mL chloramphenicol, or 0.1-0.3 µg/mL gentamicin, or similar concentrations of other antibiotics (e.g., ampicillin, polymyxin B). Host antimicrobial products such as lysozyme, defensins, and Reg proteins may be used in place of antibiotics. Bacterially-produced antimicrobial peptides, including bacteriocins and microcins may also be used.

### Temperature Stress

Bacteria are cultivated under standard growth conditions, but at higher or lower temperatures than are typical for their growth. Alternatively, bacteria are grown under standard conditions, and then subjected to cold shock or heat shock by incubation for a short period of time at low or high temperatures respectively. For example, bacteria grown at 37° C. are incubated for 1 hour at 4° C.-18° C. for cold shock or 42° C.-50° C. for heat shock.

### Starvation and Nutrient Limitation

To induce nutritional stress, bacteria are cultivated under conditions where one or more nutrients are limited. Bacteria may be subjected to nutritional stress throughout growth or shifted from a rich medium to a poor medium. Some examples of media components that are limited are carbon, nitrogen, iron, and sulfur. An example medium is M9 minimal medium (Sigma-Aldrich), which contains low glucose as the sole carbon source. Media components are also manipulated by the addition of chelators such as EDTA and deferoxamine.

### Saturation

Bacteria are grown to saturation and incubated past the saturation point for various periods of time. Alternatively, conditioned media is used to mimic saturating environments during exponential growth. Conditioned media is prepared by removing intact cells from saturated cultures by centrifugation and filtration, and conditioned media may be further treated to concentrate or remove specific components.

### Salt Stress

Bacteria are cultivated in or exposed for brief periods to medium containing NaCl, bile salts, or other salts.

### UV Stress

UV stress is achieved by cultivating bacteria under a UV lamp or by exposing bacteria to UV using an instrument such as a Stratalinker (Agilent). UV may be administered throughout the entire cultivation period, in short bursts, or for a single defined period following growth.

### Reactive Oxygen Stress

Bacteria are cultivated in the presence of sublethal concentrations of hydrogen peroxide (250-1,000 µM) to induce stress in the form of reactive oxygen species. Anaerobic bacteria are cultivated in or exposed to concentrations of oxygen that are toxic to them.

### Detergent Stress

Bacteria are cultivated in or exposed to detergent, such as sodium dodecyl sulfate (SDS) or deoxycholate.

### pH Stress

Bacteria are cultivated in or exposed for limited times to media of different pH.

### Example 12: Preparation of smEV-Free Bacteria

Bacterial samples containing minimal amounts of smEVs are prepared. smEV production is quantified (1) in complex samples of bacteria and extracellular components by NTA or TEM; or (2) following smEV purification from bacterial samples, by NTA, lipid quantification, or protein quantification.

a. Centrifugation and washing: Bacterial cultures are centrifuged at 11,000 × g to separate intact cells from supernatant (including free proteins and vesicles). The pellet is washed with buffer, such as PBS, and stored in a stable way (e.g., mixed with glycerol, flash frozen, and stored at -80° C.).

b. ATF: Bacteria and smEVs are separated by connection of a bioreactor to an ATF system. smEV-free bacteria are retained within the bioreactor, and may be further separated from residual smEVs by centrifugation and washing, as described above.

c. Bacteria are grown under conditions that are found to limit production of smEVs. Conditions that may be varied.

### Example 13: Labeling Bacterial smEVs

smEVs may be labeled in order to track their biodistribution in vivo and to quantify and track cellular localization in various preparations and in assays conducted with mammalian cells. For example, smEVs may be radio-labeled, incubated with dyes, fluorescently labeled, luminescently labeled, or labeled with conjugates containing metals and isotopes of metals.

For example, smEVs may be incubated with dyes conjugated to functional groups such as NHS-ester, click-chemistry groups, streptavidin or biotin. The labeling reaction may occur at a variety of temperatures for minutes or hours, and with or without agitation or rotation. The reaction may then be stopped by adding a reagent such as bovine serum albumin (BSA), or similar agent, depending on the protocol, and free or unbound dye molecule removed by ultra-centrifugation, filtration, centrifugal filtration, column affinity purification or dialysis. Additional washing steps involving wash buffers and vortexing or agitation may be employed to ensure complete removal of free dyes molecules such as described in Su Chul Jang et al, Small. 11, No.4, 456-461(2017).

Fluorescently labeled smEVs are detected in cells or organs, or in in vitro and/or ex vivo samples by confocal microscopy, nanoparticle tracking analysis, flow cytometry, fluorescence activated cell sorting (FACs) or fluorescent imaging system such as the Odyssey CLx LICOR (see e.g., Wiklander et al. 2015. J. Extracellular Vesicles. 4:10.3402/jev.v4.26316). Additionally, fluorescently labeled smEVs are detected in whole animals and/or dissected organs and tissues using an instrument such as the IVIS spectrum CT (Perkin Elmer) or Pearl Imager, as in H-I. Choi, et al. Experimental & Molecular Medicine. 49: e330 (2017).

smEVs may be labeled with conjugates containing metals and isotopes of metals using the protocols described above. Metal-conjugated smEVs may be administered in vivo to animals. Cells may then be harvested from organs at various time-points, and analyzed ex vivo. Alternatively, cells derived from animals, humans, or immortalized cell lines may be treated with metal-labelled smEVs in vitro and cells subsequently labelled with metal-conjugated antibodies and phenotyped using a Cytometry by Time of Flight (CyTOF) instrument such as the Helios CyTOF (Fluidigm) or imaged and analyzed using and Imaging Mass Cytometry instrument such as the Hyperion Imaging System (Fluidigm). Additionally, smEVs may be labelled with a radioisotope to track the smEVs biodistribution (see, e.g., Miller et al., Nanoscale. 2014 May 7;6(9):4928-35).

### Example 14: Transmission Electron Microscopy to Visualize Purified bacterial smEVs

smEVs are purified from bacteria batch cultures. Transmission electron microscopy (TEM) may be used to visualize purified bacterial smEVs (S. Bin Park, et al. PLoS ONE. 6(3):e17629 (2011). smEVs are mounted onto 300- or 400-mesh-size carbon-coated copper grids (Electron Microscopy Sciences, USA) for 2 minutes and washed with deionized water. smEVs are negatively stained using 2% (w/v) uranyl acetate for 20 sec -1 min. Copper grids are washed with sterile water and dried. Images are acquired using a transmission electron microscope with 100-120 kV acceleration voltage. Stained smEVs appear between 20-600 nm in diameter and are electron dense. 10-50 fields on each grid are screened.

### Example 15: Profiling smEV Composition and Content

smEVs may be characterized by any one of various methods including, but not limited to, NanoSight characterization, SDS-PAGE gel electrophoresis, Western blot, ELISA, liquid chromatography-mass spectrometry and mass spectrometry, dynamic light scattering, lipid levels, total protein, lipid to protein ratios, nucleic acid analysis and/or zeta potential.

### NanoSight Characterization of smEVs

Nanoparticle tracking analysis (NTA) is used to characterize the size distribution of purified smEVs. Purified smEV preparations are run on a NanoSight machine (Malvern Instruments) to assess smEV size and concentration.

### SDS-PAGE Gel Electrophoresis

To identify the protein components of purified smEVs, samples are run on a gel, for example a Bolt Bis-Tris Plus 4-12% gel (Thermo-Fisher Scientific), using standard techniques. Samples are boiled in 1x SDS sample buffer for 10 minutes, cooled to 4° C., and then centrifuged at 16,000 × g for 1 min. Samples are then run on a SDS-PAGE gel and stained using one of several standard techniques (e.g., Silver staining, Coomassie Blue, Gel Code Blue) for visualization of bands.

### Western Blot Analysis

To identify and quantify specific protein components of purified smEVs, smEV proteins are separated by SDS-PAGE as described above and subjected to Western blot analysis (Cvjetkovic et al., Sci. Rep. 6, 36338 (2016)) and are quantified via ELISA. smEV proteomics and Liquid Chromatography-Mass Spectrometry (LC-MS/MS) and Mass Spectrometry (MS)

Proteins present in smEVs are identified and quantified by Mass Spectrometry techniques. smEV proteins may be prepared for LC-MS/MS using standard techniques including protein reduction using dithiotreitol solution (DTT) and protein digestion using enzymes such as LysC and trypsin as described in Erickson et al, 2017 (Molecular Cell, VOLUME 65, ISSUE 2, P361-370, JANUARY 19, 2017). Alternatively, peptides are prepared as described by Liu et al. 2010 (JOURNAL OF BACTERIOLOGY, June 2010, p. 2852-2860 Vol. 192, No. 11), Kieselbach and Oscarsson 2017 (Data Brief. 2017 Feb; 10: 426-431.), Vildhede et al, 2018 (Drug Metabolism and Disposition Feb. 8, 2018). Following digestion, peptide preparations are run directly on liquid chromatography and mass spectrometry devices for protein identification within a single sample. For relative quantitation of proteins between samples, peptide digests from different samples are labeled with isobaric tags using the iTRAQ Reagent-8plex Multiplex Kit (Applied Biosystems, Foster City, CA) or TMT 10plex and 11plex Label Reagents (Thermo Fischer Scientific, San Jose, CA, USA). Each peptide digest is labeled with a different isobaric tag and then the labeled digests are combined into one sample mixture. The combined peptide mixture is analyzed by LC-MS/MS for both identification and quantification. A database search is performed using the LC-MS/MS data to identify the labeled peptides and the corresponding proteins. In the case of isobaric labeling, the fragmentation of the attached tag generates a low molecular mass reporter ion that is used to obtain a relative quantitation of the peptides and proteins present in each smEV.

Additionally, metabolic content is ascertained using liquid chromatography techniques combined with mass spectrometry. A variety of techniques exist to determine metabolomic content of various samples and are known to one skilled in the art involving solvent extraction, chromatographic separation and a variety of ionization techniques coupled to mass determination (Roberts et al 2012 Targeted Metabolomics. Curr Protoc Mol Biol. 30: 1-24; Dettmer et al 2007, Mass spectrometry-based metabolomics. Mass Spectrom Rev. 26(1):51-78). As a non-limiting example, a LC-MS system includes a 4000 QTRAP triple quadrupole mass spectrometer (AB SCIEX) combined with 1100 Series pump (Agilent) and an HTS PAL autosampler (Leap Technologies). Media samples or other complex metabolic mixtures (~10 µL) are extracted using nine volumes of 74.9:24.9:0.2 (v/v/v) acetonitrile/methanol/formic acid containing stable isotope-labeled internal standards (valine-d8, Isotec; and phenylalanine-d8, Cambridge Isotope Laboratories). Standards may be adjusted or modified depending on the metabolites of interest. The samples are centrifuged (10 minutes, 9,000 g, 4° C.), and the supernatants (10 µL) are submitted to LCMS by injecting the solution onto the HILIC column (150 × 2.1 mm, 3 µm particle size). The column is eluted by flowing a 5% mobile phase [10 mM ammonium formate, 0.1% formic acid in water] for 1 minute at a rate of 250uL/minute followed by a linear gradient over 10 minutes to a solution of 40% mobile phase [acetonitrile with 0.1% formic acid]. The ion spray voltage is set to 4.5 kV and the source temperature is 450° C.

The data are analyzed using commercially available software like Multiquant 1.2 from AB SCIEX for mass spectrum peak integration. Peaks of interest should be manually curated and compared to standards to confirm the identity of the peak. Quantitation with appropriate standards is performed to determine the number of metabolites present in the initial media, and after bacterial conditioning. A non-targeted metabolomics approach may also be used using metabolite databases, such as but not limited to the NIST database, for peak identification.

### Dynamic Light Scattering (DLS)

DLS measurements, including the distribution of particles of different sizes in different smEV preparations are taken using instruments such as the DynaPro NanoStar (Wyatt Technology) and the Zetasizer Nano ZS (Malvern Instruments).

### Lipid Levels

Lipid levels are quantified using FM4-64 (Life Technologies), by methods similar to those described by A.J. McBroom et al. J Bacteriol 188:5385-5392. and A. Frias, et al. Microb Ecol. 59:476-486 (2010). Samples are incubated with FM4-64 (3.3 µg/mL in PBS for 10 minutes at 37° C. in the dark). After excitation at 515 nm, emission at 635 nm is measured using a Spectramax M5 plate reader (Molecular Devices). Absolute concentrations are determined by comparison of unknown samples to standards (such as palmitoyloleoylphosphatidylglycerol (POPG) vesicles) of known concentrations. Lipidomics can be used to identify the lipids present in the smEVs.

### Total Protein

Protein levels are quantified by standard assays such as the Bradford and BCA assays. The Bradford assays are run using Quick Start Bradford 1x Dye Reagent (Bio-Rad), according to manufacturer’s protocols. BCA assays are run using the Pierce BCA Protein Assay Kit (Thermo-Fisher Scientific). Absolute concentrations are determined by comparison to a standard curve generated from BSA of known concentrations. Alternatively, protein concentration can be calculated using the Beer-Lambert equation using the sample absorbance at 280 nm (A280) as measured on a Nanodrop spectrophotometer (Thermo-Fisher Scientific).In addition, proteomics may be used to identify proteins in the sample.

### Lipid:Protein Ratios

Lipid:protein ratios are generated by dividing lipid concentrations by protein concentrations. These provide a measure of the purity of vesicles as compared to free protein in each preparation.

### Nucleic Acid Analysis

Nucleic acids are extracted from smEVs and quantified using a Qubit fluorometer. Size distribution is assessed using a BioAnalyzer and the material is sequenced.

### Zeta Potential

The zeta potential of different preparations are measured using instruments such as the Zetasizer ZS (Malvern Instruments).

### Example 16: In Vitro Screening of smEVs for Enhanced Activation of Dendritic Cells

In vitro immune responses are thought to simulate mechanisms by which immune responses are induced in vivo. Briefly, PBMCs are isolated from heparinized venous blood from healthy donors by gradient centrifugation using Lymphoprep (Nycomed, Oslo, Norway), or from mouse spleens or bone marrow using the magnetic bead-based Human Blood Dendritic cell isolation kit (Miltenyi Biotech, Cambridge, MA). Using anti-human CD14 mAb, the monocytes are purified by Moflo and cultured in cRPMI at a cell density of 5e5 cells/ml in a 96-well plate (Costar Corp) for 7 days at 37° C. For maturation of dendritic cells, the culture is stimulated with 0.2 ng/mL IL-4 and 1000 U/ml GM-CSF at 37° C. for one week. Alternatively, maturation is achieved through incubation with recombinant GM-CSF for a week, or using other methods known in the art. Mouse DCs can be harvested directly from spleens using bead enrichment or differentiated from hematopoietic stem cells. Briefly, bone marrow may be obtained from the femurs of mice. Cells are recovered and red blood cells lysed. Stem cells are cultured in cell culture medium in 20 ng/ml mouse GMCSF for 4 days. Additional medium containing 20 ng/ml mouse GM-CSF is added. On day 6 the medium and non-adherent cells are removed and replaced with fresh cell culture medium containing 20 ng/ml GMCSF. A final addition of cell culture medium with 20 ng/ml GM-CSF is added on day 7. On day10, non-adherent cells are harvested and seeded into cell culture plates overnight and stimulated as required. Dendritic cells are then treated with various doses of smEVs with or without antibiotics. For example, 25-75 µg/mL smEVs for 24 hours with antibiotics. smEV compositions tested may include smEVs from a single bacterial species or strain, or a mixture of smEVs from one or more genus, 1 or more species, or 1 or more strains (e.g., one or more strains within one species). PBS is included as a negative control and LPS, anti-CD40 antibodies, and/or smEVs are used as positive controls. Following incubation, DCs are stained with anti CD11b, CD11c, CD103, CD8a, CD40, CD80, CD83, CD86, MHCI and MHCII, and analyzed by flow cytometry. DCs that are significantly increased in CD40, CD80, CD83, and CD86 as compared to negative controls are considered to be activated by the associated bacterial smEV composition. These experiments are repeated three times at minimum.

To screen for the ability of smEV-activated epithelial cells to stimulate DCs, the above protocol is followed with the addition of a 24-hour epithelial cell smEV co-culture prior to incubation with DCs. Epithelial cells are washed after incubation with smEVs and are then co-cultured with DCs in an absence of smEVs for 24 hours before being processed as above. Epithelial cell lines may include Int407, HEL293, HT29, T84 and CACO2.

As an additional measure of DC activation, 100 µl of culture supernatant is removed from wells following 24-hour incubation of DCs with smEVs or smEV-treated epithelial cells and is analyzed for secreted cytokines, chemokines, and growth factors using the multiplexed Luminex Magpix. Kit (EMD Millipore, Darmstadt, Germany). Briefly, the wells are pre-wet with buffer, and 25 µl of 1x antibody-coated magnetic beads are added and 2x 200 µl of wash buffer are performed in every well using the magnet. 50 µl of Incubation buffer, 50 µl of diluent and 50 µl of samples are added and mixed via shaking for 2 hrs at room temperature in the dark. The beads are then washed twice with 200 µl wash buffer. 100 µl of 1X biotinylated detector antibody is added and the suspension is incubated for 1 hour with shaking in the dark. Two, 200 µl washes are then performed with wash buffer. 100 µl of 1x SAV-RPE reagent is added to each well and is incubated for 30 min at RT in the dark. Three 200 µl washes are performed and 125 µl of wash buffer is added with 2-3 min shaking occurs. The wells are then submitted for analysis in the Luminex xMAP system.

Standards allow for careful quantitation of the cytokines including GM-CSF, IFN-g, IFN-a, IFN-B, IL-1a, IL-1B, IL-2, IL-4, IL-5, IL-6, IL-8, IL-10, IL-13, IL-12 (p40/p70), IL-17A, IL-17F, IL-21, IL-22 IL-23, IL-25, IP-10, KC, MCP-1, MIG, MIP1a, TNFa, and VEGF. These cytokines are assessed in samples of both mouse and human origin. Increases in these cytokines in the bacterial treated samples indicate enhanced production of proteins and cytokines from the host. Other variations on this assay examining specific cell types ability to release cytokines are assessed by acquiring these cells through sorting methods and are recognized by one of ordinary skill in the art. Furthermore, cytokine mRNA is also assessed to address cytokine release in response to an smEV composition.

This DC stimulation protocol may be repeated using combinations of purified smEVs and live bacterial strains to maximize immune stimulation potential.

### Example 17: In Vitro Detection of smEVs in Antigen-presenting Cells

Dendritic cells in the lamina propria constantly sample live bacteria, dead bacteria, and microbial products in the gut lumen by extending their dendrites across the gut epithelium, which is one way that smEVs produced by bacteria in the intestinal lumen may directly stimulate dendritic cells. The following methods represent a way to assess the differential uptake of smEVs by antigen-presenting cells. Optionally, these methods may be applied to assess immunomodulatory behavior of smEVs administered to a patient.

Dendritic cells (DCs) are isolated from human or mouse bone marrow, blood, or spleens according to standard methods or kit protocols (e.g., Inaba K, Swiggard WJ, Steinman RM, Romani N, Schuler G, 2001. Isolation of dendritic cells. Current Protocols in Immunology. Chapter 3:Unit3.7).

To evaluate smEV entrance into and/or presence in DCs, 250,000 DCs are seeded on a round cover slip in complete RPMI-1640 medium and are then incubated with smEVs from single bacterial strains or combinations smEVs at various ratios. Purified smEVs may be labeled with fluorochromes or fluorescent proteins. After incubation for various timepoints (e.g., 1 hour, 2 hours), the cells are washed twice with ice-cold PBS and detached from the plate using trypsin. Cells are either allowed to remain intact or are lysed. Samples are then processed for flow cytometry. Total internalized smEVs are quantified from lysed samples, and percentage of cells that uptake smEVs is measured by counting fluorescent cells. The methods described above may also be performed in substantially the same manner using macrophages or epithelial cell lines (obtained from the ATCC) in place of DCs.

### Example 18: Determining the Biodistribution of smEVs When Delivered Orally To Mice

Wild-type mice (e.g., C57BL/6 or BALB/c) are orally inoculated with the smEV composition of interest to determine the in vivo biodistibution profile of purified smEVs. smEVs are labeled to aide in downstream analyses. Alternatively, mice with some immune disorder (e.g., systemic lupus erythematosus, experimental autoimmune encephalomyelitis, NASH) may be studied for in vivo distribution of smEVs over a given time-course.

Mice can receive a single dose of the smEV (e.g., 25-100 µg) or several doses over a defined time course (25-100 µg). Alternatively, smEVs dosages may be administered based on particle count (e.g., 7e+08 to 6e+11 particles). Mice are housed under specific pathogen-free conditions following approved protocols. Alternatively, mice may be bred and maintained under sterile, germ-free conditions. Blood, stool, and other tissue samples can be taken at appropriate time points.

The mice are humanely sacrificed at various time points (i.e., hours to days) post administration of the smEV compositions, and a full necropsy under sterile conditions is performed. Following standard protocols, lymph nodes, adrenal glands, liver, colon, small intestine, cecum, stomach, spleen, kidneys, bladder, pancreas, heart, skin, lungs, brain, and other tissue of interest are harvested and are used directly or snap frozen for further testing. The tissue samples are dissected and homogenized to prepare single-cell suspensions following standard protocols known to one skilled in the art. The number of smEVs present in the sample is then quantified through flow cytometry. Quantification may also proceed with use of fluorescence microscopy after appropriate processing of whole mouse tissue (Vankelecom H., Fixation and paraffin-embedding of mouse tissues for GFP visualization, Cold Spring Harb. Protoc., 2009). Alternatively, the animals may be analyzed using live-imaging according to the smEV labeling technique.

Biodistribution may be performed in mouse models of autoimmunity such as but not limited to EAE and DTH (see, e.g., Turjeman et al., PLoS One 10(7): e0130442 (20105).

### Example 19: Manufacturing Conditions

Enriched media is used to grow and prepare the bacteria for in vitro and in vivo use and, ultimately, for pmEV and smEV preparations. For example, media may contain sugar, yeast extracts, plant-based peptones, buffers, salts, trace elements, surfactants, anti-foaming agents, and vitamins. Composition of complex components such as yeast extracts and peptones may be undefined or partially defined (including approximate concentrations of amino acids, sugars etc.). Microbial metabolism may be dependent on the availability of resources such as carbon and nitrogen. Various sugars or other carbon sources may be tested. Alternatively, media may be prepared and the selected bacterium grown as shown by Saarela et al., J. Applied Microbiology. 2005. 99: 1330-1339, which is hereby incorporated by reference. Influence of fermentation time, cryoprotectant and neutralization of cell concentrate on freeze-drying survival, storage stability, and acid and bile exposure of the selected bacterium produced without milk-based ingredients.

At large scale, the media is sterilized. Sterilization may be accomplished by Ultra High Temperature (UHT) processing. The UHT processing is performed at very high temperature for short periods of time. The UHT range may be from 135-180° C. For example, the medium may be sterilized from between 10 to 30 seconds at 135° C.

Inoculum can be prepared in flasks or in smaller bioreactors and growth is monitored. For example, the inoculum size may be between approximately 0.5 and 3% of the total bioreactor volume. Depending on the application and need for material, bioreactor volume can be at least 2 L, 10 L, 80 L, 100 L, 250 L, 1000 L, 2500 L, 5000 L, 10,000 L.

Before the inoculation, the bioreactor is prepared with medium at desired pH, temperature, and oxygen concentration. The initial pH of the culture medium may be different that the process set-point. pH stress may be detrimental at low cell centration; the initial pH could be between pH 7.5 and the process set-point. For example, pH may be set between 4.5 and 8.0. During the fermentation, the pH can be controlled through the use of sodium hydroxide, potassium hydroxide, or ammonium hydroxide. The temperature may be controlled from 25° C. to 45° C., for example at 37° C. Anaerobic conditions are created by reducing the level of oxygen in the culture broth from around 8 mg/L to 0 mg/L. For example, nitrogen or gas mixtures (N2, CO2, and H2) may be used in order to establish anaerobic conditions. Alternatively, no gases are used and anaerobic conditions are established by cells consuming remaining oxygen from the medium. Depending on strain and inoculum size, the bioreactor fermentation time can vary. For example, fermentation time can vary from approximately 5 hours to 48 hours.

Reviving microbes from a frozen state may require special considerations. Production medium may stress cells after a thaw; a specific thaw medium may be required to consistently start a seed train from thawed material. The kinetics of transfer or passage of seed material to fresh medium, for the purposes of increasing the seed volume or maintaining the microbial growth state, may be influenced by the current state of the microbes (ex. exponential growth, stationary growth, unstressed, stressed).

Inoculation of the production fermenter(s) can impact growth kinetics and cellular activity. The initial state of the bioreactor system must be optimized to facilitate successful and consistent production. The fraction of seed culture to total medium (e.g., a percentage) has a dramatic impact on growth kinetics. The range may be 1-5% of the fermenter’s working volume. The initial pH of the culture medium may be different from the process set-point. pH stress may be detrimental at low cell concentration; the initial pH may be between pH 7.5 and the process set-point. Agitation and gas flow into the system during inoculation may be different from the process set-points. Physical and chemical stresses due to both conditions may be detrimental at low cell concentration.

Process conditions and control settings may influence the kinetics of microbial growth and cellular activity. Shifts in process conditions may change membrane composition, production of metabolites, growth rate, cellular stress, etc. Optimal temperature range for growth may vary with strain. The range may be 20-40° C. Optimal pH for cell growth and performance of downstream activity may vary with strain. The range may be pH 5-8. Gasses dissolved in the medium may be used by cells for metabolism. Adjusting concentrations of O2, CO2, and N₂ throughout the process may be required. Availability of nutrients may shift cellular growth. Microbes may have alternate kinetics when excess nutrients are available.

The state of microbes at the end of a fermentation and during harvesting may impact cell survival and activity. Microbes may be preconditioned shortly before harvest to better prepare them for the physical and chemical stresses involved in separation and downstream processing. A change in temperature (often reducing to 20-5° C.) may reduce cellular metabolism, slowing growth (and/or death) and physiological change when removed from the fermenter. Effectiveness of centrifugal concentration may be influenced by culture pH. Raising pH by 1-2 points can improve effectiveness of concentration but can also be detrimental to cells. Microbes may be stressed shortly before harvest by increasing the concentration of salts and/or sugars in the medium. Cells stressed in this way may better survive freezing and lyophilization during downstream.

Separation methods and technology may impact how efficiently microbes are separated from the culture medium. Solids may be removed using centrifugation techniques. Effectiveness of centrifugal concentration can be influenced by culture pH or by the use of flocculating agents. Raising pH by 1-2 points may improve effectiveness of concentration but can also be detrimental to cells. Microbes may be stressed shortly before harvest by increasing the concentration of salts and/or sugars in the medium. Cells stressed in this way may better survive freezing and lyophilization during downstream. Additionally, Microbes may also be separated via filtration. Filtration is superior to centrifugation techniques for purification if the cells require excessive g-minutes to successfully centrifuge. Excipients can be added before after separation. Excipients can be added for cryo protection or for protection during lyophilization. Excipients can include, but are not limited to, sucrose, trehalose, or lactose, and these may be alternatively mixed with buffer and anti-oxidants. Prior to lyophilization, droplets of cell pellets mixed with excipients are submerged in liquid nitrogen.

Harvesting can be performed by continuous centrifugation. Product may be resuspended with various excipients to a desired final concentration. Excipients can be added for cryo protection or for protection during lyophilization. Excipients can include, but are not limited to, sucrose, trehalose, or lactose, and these may be alternatively mixed with buffer and anti-oxidants. Prior to lyophilization, droplets of cell pellets mixed with excipients are submerged in liquid nitrogen.

Lyophilization of material, including live bacteria, vesicles, or other bacterial derivative includes a freezing, primary drying, and secondary drying phase. Lyophilization begins with freezing. The product material may or may not be mixed with a lyoprotectant or stabilizer prior to the freezing stage. A product may be frozen prior to the loading of the lyophilizer, or under controlled conditions on the shelf of the lyophilizer. During the next phase, the primary drying phase, ice is removed via sublimation. Here, a vacuum is generated and an appropriate amount of heat is supplied to the material. The ice will sublime while keeping the product temperature below freezing, and below the material’s critical temperature (T_{c}). The temperature of the shelf on which the material is loaded and the chamber vacuum can be manipulated to achieve the desired product temperature. During the secondary drying phase, product-bound water molecules are removed. Here, the temperature is generally raised higher than in the primary drying phase to break any physico-chemical interactions that have formed between the water molecules and the product material. After the freeze-drying process is complete, the chamber may be filled with an inert gas, such as nitrogen. The product may be sealed within the freeze dryer under dry conditions, in a glass vial or other similar container, preventing exposure to atmospheric water and contaminates.

### Example 20: smEV and pmEV Preparation

smEVs: Downstream processing of smEVs begins immediately following harvest of the bioreactor. Centrifugation at 20,000 g is used to remove the cells from the broth. The resulting supernatant is clarified using 0.22 µm filter. The smEVs are concentrated and washed using tangential flow filtration (TFF) with flat sheet cassettes ultrafiltration (UF) membranes with 100 kDa molecular weight cutoff (MWCO). Diafiltration (DF) is used to washout small molecules and small proteins using 5 volumes of phosphate buffer solution (PBS). The retentate from TFF is spun down in an ultracentrifuge at 200,000 g for 1 hour to form a pellet rich in smEVs called a high-speed pellet (HSP). The pellet is resuspended with minimal PBS and a gradient was prepared with optiprep™ density gradient medium and ultracentrifuged at 200,000 g for 16 hours. Of the resulting fractions, 2 middle bands contain smEVs. The fractions are washed with 15 fold PBS and the smEVs are spun down at 200,000 g for 1 hr to create the fractionated HSP or fHSP. It is subsequently resuspended with minimal PBS, pooled, and analyzed for particles per mL and protein content. Dosing is prepared from the particle / mL count to achieve desired concentration. The smEVs are characterized using a NanoSight NS300 by Malvern Panalytical in scatter mode using the 532 nm laser.

### pmEVs

Cell pellets are removed from freezer and placed on ice. Pellet weights are noted.

Cold 100 mM Tris-HCl pH 7.5 is added to the frozen pellets and the pellets are thawed rotating at 4° C.

10 mg/ml DNase stock is added to the thawed pellets to a final concentration of 1 mg/mL.

The pellets are incubated on the inverter for 40 min at RT (room temperature).

The sample is filtered in a 70 um cell strainer before running through the Emulsiflex.

The samples ae lysed using the Emulsiflex with 8 discrete cycles at 22,000 psi.

To remove the cellular debris from the lysed sample, the sample is centrifuged at 12,500 × g, 15 min, 4° C.

The sample is centrifuged two additional times at 12,500 × g, 15 min, 4° C., each time moving the supernatant to a fresh tube.

To pellet the membrane proteins, the sample is centrifuged at 120,000 × g, 1 hr, 4° C.

The pellet is resuspended in 10 mL ice-cold 0.1 M sodium carbonate pH 11. The sample is incubated on the inverter at 4° C. for 1 hour.

The sample is centrifuged at 120,000 × g, 1 hr, 4° C.

10 mL 100 mM Tris-HCl pH 7.5 is added to pellet and incubate O/N (overnight) at 4° C.

The pellet is resuspended and the sample was centrifuged at 120,000xg for 1 hour at 4° C.

The supernatant is discarded and the pellet was resuspended in a minimal volume of PBS.

Dosing pmEVs is based on particle counts, as assessed by Nanoparticle Tracking Analysis (NTA) using a NanoSight NS300 (Malvern Panalytical) according to manufacturer instructions. Counts for each sample are based on at least three videos of 30 sec duration each, counting 40-140 particles per frame.

Gamma irradiation: For gamma irradiation, pmEVs are prepared in frozen form and gamma irradiated on dry ice at 25 kGy radiation dose; whole microbe lyophilized powder is gamma irradiated at ambient temperature at 17.5 kGy radiation dose.

Lyophilization: Samples are placed in lyophilization equipment and frozen at -45° C. The lyophilization cycle included a hold step at -45° C. for 10 min. The vacuum begins and is set to 100 mTorr and the sample was held at -45° C. for another 10 min. Primary drying begins with a temperature ramp to -25° C. over 300 minutes and it is held at this temperature for 4630 min. Secondary drying starts with a temperature ramp to 20° C. over 200 min while the vacuum is decreased to 20 mTorr. It is held at this temperature and pressure for 1200 min. The final step increases the temperature from 20 to 25° C. where it remains at a vacuum of 20 mTorr for 10 min.

### Example 21: Oral Delivery of Non-Live Preparation of Veillonella Parvula Modulates CNS Inflammation Via the Small Intestinal Axis: a Novel Approach to Treating Neuroinflammatory Diseases

Rationale: The small intestinal axis (SINTAX) is a network of functional anatomical connections between the gut and the rest of the body. SINTAX can be targeted with orally-delivered, gut-restricted agents that have systemic anti-inflammatory effects, including in the CNS. A pharmaceutical preparation of a gamma irradiated (G.I.) single strain of Veillonella parvula, Veillonella parvula Strain A (Strain A), isolated from the ileum of a human donor, was studied. During manufacturing, Veillonella parvula Strain A (Strain A) is gamma irradiated after fermentation making it a non-live bacterial product. G.I. Strain A has pharmacological activity via SINTAX in T cell-mediated murine inflammation models, reducing peripheral inflammation without toxicity. We investigated whether G.I. Strain A was also efficacious in a model of chronic neuroinflammation and demyelination. This might suggest benefit in multiple sclerosis (MS).

Methods: Orally-administered G.I. Strain A was tested in the PLP-induced relapsing-remitting Experimental Autoimmune Encephalomyelitis (EAE) SJL mouse model.

Results: Oral treatment with G.I. Strain A reduced disease severity and incidence of relapse compared to vehicle-treated controls in EAE. Prophylactic G.I. Strain A treatment reduced mean maximum score (MMS) in both the acute phase between days 7-18 (G.I. Strain A 2.57 ± 1.07, vehicle 3.25 ± 1.49) as well as the relapse period from day 19-42 (G.I. Strain A 2.07 ± 0.88, vehicle 3.04 ± 1.74, p=0.0407). In addition, therapeutic G.I. Strain A treatment reduced MMS in the relapse period from day 18-42 (G.I. Strain A 1.60 ± 0.87, vehicle 2.57 ± 0.86, p=0.0069). In histopathological analysis of spinal cord sections, G.I. Strain A reduced the infiltration of inflammatory cells into the CNS. Demyelination in the spinal cord was also reduced. Transcriptional profiling of duodenal tissue revealed that G.I. Strain A upregulated genes in T and B cell pathways that resolve inflammation as well as genes associated with intestinal homeostasis.

Conclusions: Our results indicate that SINTAX is a portal to the CNS by immune signaling from the gut. This is a striking finding that suggests a new pathway of biological control of neuroinflammation and type of medicine for its treatment. The efficacy of this non-live bacterial product is evidence that this is a result of a direct pharmacological interaction with host cells in the gut and is not the result of modification of the gut microbiota. The transcriptional profiling results are an insight into the mechanisms of this novel biology and pharmacology.

### INCORPORATION BY REFERENCE

All publications or patent applications mentioned herein are hereby incorporated by reference in their entirety as if each individual publication or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A pharmaceutical composition for the treatment of a neuroinflammatory disease, a neurodegenerative disease, a neuromuscular disease, and/or a psychiatric disorder, the pharmaceutical composition comprising Veillonella parvula bacteria, wherein the Veillonella parvula is a strain comprising at least 90% genomic, 16S, and/or CRISPR sequence identity to the nucleotide sequence of the Veillonella parvula Strain A (ATCC Deposit Number PTA-125691).

2. The pharmaceutical composition of claim 1, wherein the Veillonella parvula is a strain comprising at least 95% genomic, 16S, and/or CRISPR sequence identity to the nucleotide sequence of the Veillonella parvula Strain A (ATCC Deposit Number PTA-125691).

3. The pharmaceutical composition of claim 1, wherein the Veillonella parvula is a strain comprising at least 99% genomic, 16S, and/or CRISPR sequence identity to the nucleotide sequence of the Veillonella parvula Strain A (ATCC Deposit Number PTA-125691).

4. The pharmaceutical composition of claim 1, wherein the Veillonella parvula is a strain comprising at least 99% 16S sequence identity to the nucleotide sequence of the Veillonella parvula Strain A (ATCC Deposit Number PTA-125691).

5. The pharmaceutical composition of claim 1, wherein the Veillonella parvula is Veillonella parvula Strain A (ATCC Deposit Number PTA-125691).

6. The pharmaceutical composition of any one of claims 1-5, wherein at least 50% of the bacteria in the pharmaceutical composition are Veillonella parvula Strain A.

7. The pharmaceutical composition of any one of claims 1-6, wherein at least 90% of the bacteria in the pharmaceutical composition are Veillonella parvula Strain A.

8. The pharmaceutical composition of any one of claims 1-7, wherein substantially all of the bacteria in the pharmaceutical composition are Veillonella parvula Strain A.

9. The pharmaceutical composition of any one of claims 1-8, wherein the pharmaceutical composition comprises at least 1 ×10⁶ colony forming units (CFUs) of Veillonella parvula Strain A.

10. The pharmaceutical composition of any one of claims 1-9, wherein the pharmaceutical composition comprises at least 1 ×10⁷ colony forming units (CFUs) of Veillonella parvula Strain A.

11. The pharmaceutical composition of any one of claims 1-10, wherein the pharmaceutical composition comprises at least 1 ×10⁸ colony forming units (CFUs) of Veillonella parvula Strain A.

12. The pharmaceutical composition of any one of claims 1-11, wherein the pharmaceutical composition comprises live bacteria.

13. The pharmaceutical composition of any one of claims 1-11, wherein the pharmaceutical composition comprises attenuated bacteria.

14. The pharmaceutical composition of any one of claims 1-11, wherein the pharmaceutical composition comprises killed bacteria.

15. The pharmaceutical composition of any one of claims 1-14, wherein the pharmaceutical composition comprises lyophilized bacteria.

16. The pharmaceutical composition of any one of claims 1-15, wherein the pharmaceutical composition comprises irradiated bacteria.

17. The pharmaceutical composition of claim 16, wherein the pharmaceutical composition comprises gamma irradiated bacteria.

18. A pharmaceutical composition for the treatment of a neuroinflammatory disease, a neurodegenerative disease, a neuromuscular disease, and/or a psychiatric disorder, the pharmaceutical composition comprising isolated extracellular vesicles (mEVs) produced from Veillonella parvula, wherein the Veillonella parvula is a strain comprising at least 90% genomic, 16S, and/or CRISPR sequence identity to the nucleotide sequence of the Veillonella parvula Strain A (ATCC Deposit Number PTA-125691).

19. The pharmaceutical composition of claim 18, wherein the Veillonella parvula is a strain comprising at least 95% genomic, 16S, and/or CRISPR sequence identity to the nucleotide sequence of the Veillonella parvula Strain A (ATCC Deposit Number PTA-125691).

20. The pharmaceutical composition of claim 18, wherein the Veillonella parvula is a strain comprising at least 99% genomic, 16S, and/or CRISPR sequence identity to the nucleotide sequence of the Veillonella parvula Strain A (ATCC Deposit Number PTA-125691).

21. The pharmaceutical composition of claim 18, wherein the Veillonella parvula is a strain comprising at least 99% 16S sequence identity to the nucleotide sequence of the Veillonella parvula Strain A (ATCC Deposit Number PTA-125691).

22. The pharmaceutical composition of claim 18, wherein the Veillonella parvula is Veillonella parvula Strain A (ATCC Deposit Number PTA-125691).

23. The pharmaceutical composition of claim 18, wherein at least 75%, at least 80%, at least 85%, at least 90%, at least 95%, or at least 99% of the pharmaceutical composition is mEVs.

24. The pharmaceutical composition of any one of claims 18-23, wherein the composition comprises secreted mEVs (smEVs).

25. The pharmaceutical composition of any one of claims 18-23, wherein the composition comprises processed mEVs (pmEVs).

26. The pharmaceutical composition of any one of claims 18-23, wherein the mEVs comprise pmEVs and the pmEVs are produced from bacteria that have been gamma irradiated, UV irradiated, heat inactivated, acid treated or oxygen sparged.

27. The pharmaceutical composition of any one of claims 18-23, wherein the mEVs comprise pmEVs and the pmEVs are produced from live bacteria.

28. The pharmaceutical composition of any one of claims 18-27, wherein the mEVs are lyophilized (e.g., the lyophilized product further comprises a pharmaceutically acceptable excipient).

29. The pharmaceutical composition of any one of claims 18-28, wherein the mEVs are gamma irradiated.

30. The pharmaceutical composition of any one of claims 18-28, wherein the mEVs are UV irradiated.

31. The pharmaceutical composition of any one of claims 18-28, wherein the mEVs are heat inactivated (e.g., at 50° C. for two hours or at 90° C. for two hours).

32. The pharmaceutical composition of any one of claims 18-28, wherein the mEVs are acid treated.

33. The pharmaceutical composition of any one of claims 18-28, wherein the mEVs are oxygen sparged (e.g., at 0.1 vvm for two hours).

34. The pharmaceutical composition of any one of claims 18-33, wherein the dose of mEVs is about 2×10⁶ to about 2×10¹⁶ particles (e.g., wherein particle count is determined by NTA (nanoparticle tracking analysis)).

35. The pharmaceutical composition of any one of claims 18-34, wherein the dose of mEVs is about 5 mg to about 900 mg total protein (e.g., wherein total protein is determined by Bradford assay or BCA).

36. A pharmaceutical composition comprising Veillonella parvula microbial extracellular vesicles (mEVs) and Veillonella parvula bacteria, wherein the Veillonella parvula is a strain comprising at least 90% genomic, 16S, and/or CRISPR sequence identity to the nucleotide sequence of the Veillonella parvula Strain A (ATCC Deposit Number PTA-125691).

37. The pharmaceutical composition of claim 36, wherein the Veillonella parvula is a strain comprising at least 95% genomic, 16S, and/or CRISPR sequence identity to the nucleotide sequence of the Veillonella parvula Strain A (ATCC Deposit Number PTA-125691).

38. The pharmaceutical composition of claim 36, wherein the Veillonella parvula is a strain comprising at least 99% genomic, 16S, and/or CRISPR sequence identity to the nucleotide sequence of the Veillonella parvula Strain A (ATCC Deposit Number PTA-125691).

39. The pharmaceutical composition of claim 36, wherein the Veillonella parvula is a strain comprising at least 99% 16S sequence identity to the nucleotide sequence of the Veillonella parvula Strain A (ATCC Deposit Number PTA-125691).

40. The pharmaceutical composition of claim 36, wherein the Veillonella parvula is Veillonella parvula Strain A (ATCC Deposit Number PTA-125691).

41. The pharmaceutical composition of any one of claims 36-40, wherein at least, about, or no more than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the total particles in the pharmaceutical composition are Veillonella parvula mEVs.

42. The pharmaceutical composition of any one of claims 36-40, wherein at least, about, or no more than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the total particles in the pharmaceutical composition are Veillonella parvula bacteria particles.

43. The pharmaceutical composition of any one of claims 36-40, wherein at least, about, or no more than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the total proteins in the pharmaceutical composition are Veillonella parvula mEVs.

44. The pharmaceutical composition of any one of claims 36-40, wherein at least, about, or no more than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the total proteins in the pharmaceutical composition are Veillonella parvula bacteria proteins.

45. The pharmaceutical composition of any one of claims 36-40, wherein at least, about, or no more than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the total lipids in the pharmaceutical composition are Veillonella parvula mEVs.

46. The pharmaceutical composition of any one of claims 36-40, wherein at least, about, or no more than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the total lipids in the pharmaceutical composition are Veillonella parvula bacteria lipids.

47. The pharmaceutical composition of any one of claims 1-46, wherein the pharmaceutical composition is for the treatment of a neuroinflammatory disease.

48. The pharmaceutical composition of any one of claims 1-46, wherein the pharmaceutical composition is for the treatment of a neurodegenerative disease.

49. The pharmaceutical composition of any one of claims 1-46, wherein the pharmaceutical composition is for the treatment of a neuromuscular disease.

50. The pharmaceutical composition of any one of claims 1-46, wherein the pharmaceutical composition is for the treatment of a psychiatric disorder.

51. The pharmaceutical composition of any one of claims 1-50, wherein the pharmaceutical composition is for the treatment of a disease selected from, encephalitis, encephalomyelitis, meningitis, Guillain-Barre syndrome, neuromyotonia, narcolepsy, multiple sclerosis, myelitis, schizophrenia, acute disseminated encephalomyelitis (ADEM), accute optic neuritis (AON), transverse myelitis, neuromyelitis optica (NMO), Alzheimer’s disease, Parkinson’s disease, amyotrophic lateral sclerosis, frontotemporal lobar dementia, optic neuritis, neuromyelitis optica spectrum disorder (NMOSD), autoimmune encephalitis, anti-NMDA receptor encephalitis, Rasmussen’s encephalitis, acute necrotizing encephalopathy of childhood (ANEC), opsoclonus-myoclonus ataxia syndrome, traumatic brain injury, Huntington’s disease, depression, anxiety, migraine, myasthenia gravis, acute ischemic stroke, epilepsy, synucleinopathies, frontotemporal dementia, progressive nonfluent aphasia, semantic dementia, Nodding syndrome, cerebral ischemia, neuropathic pain, autism spectrum disorder, fibromyalgia syndrome, progressive supranuclear palsy, corticobasal degeneration, systemic lupus erythematosus, prion disease, motor neuron diseases (MND), spinocerebellar ataxia, spinal muscular atrophy, dystonia, idiopathicintracranial hypertension, nervous system disease, central nervous system disease, peripheral nervous system disease, movement disorders, encephalopathy, peripheral neuropathy, and post-operative cognitive dysfunction.

52. The pharmaceutical composition of any one of claims 1-51, whereon the pharmaceutical composition is for the treatment of a disease selected from encephalitis, encephalomyelitis, meningitis, multiple sclerosis, schizophrenia, acute disseminated encephalomyelitis (ADEM), accute optic neuritis (AON), transverse myelitis, neuromyelitis optica (NMO), Alzheimer’s disease, Parkinson’s disease, amyotrophic lateral sclerosis, frontotemporal lobar dementia, traumatic brain injury, Huntington’s disease, depression, anxiety, migraine, acute ischemic stroke, epilepsy, synucleinopathies, semantic dementia, cerebral ischemia, neuropathic pain, autism spectrum disorder, peripheral neuropathy, motor neuron diseases (MND), spinocerebellar ataxia, spinal muscular atrophy, and fibromyalgia syndrome.

53. The pharmaceutical composition of any one of claims 1-52, wherein the pharmaceutical composition reduces inflammation, optionally neuroinflammation.

54. The pharmaceutical composition of any one of claims 1-53, wherein the pharmaceutical composition induces an immune response and/or activates innate antigen presenting cells.

55. The pharmaceutical composition of any one of claims 1-54, wherein the pharmaceutical composition is formulated for oral, rectal, sublingual, intradermal, intravenous, intraperitoneal, or subcutaneous administration.

56. The pharmaceutical composition of any one of claims 1-55 wherein the pharmaceutical composition has one or more beneficial immune effects outside the gastrointestinal tract, e.g., when orally administered.

57. The pharmaceutical composition of any one of claims 1-56, wherein the pharmaceutical composition modulates immune effects outside the gastrointestinal tract in the subject, e.g., when orally administered.

58. The pharmaceutical composition of any one of claims 1-57, wherein the pharmaceutical composition comprises a solid dose form.

59. The pharmaceutical composition of claim 58, wherein the solid dose form comprises a tablet, a minitablet, a capsule, a pill, or a powder, or a combination of the foregoing.

60. The pharmaceutical composition of claim 58 or 59 wherein the solid dose form further comprises a pharmaceutically acceptable excipient.

61. The pharmaceutical composition of any one of claims 58-60, wherein the solid dose form comprises an enteric coating.

62. The pharmaceutical composition of any one of claims 58-61, wherein the solid dose form is for oral administration.

63. The pharmaceutical composition of any one of claims 1-57, wherein the pharmaceutical composition comprises a suspension.

64. The pharmaceutical composition of claim 63, wherein the suspension is for oral administration (e.g., the suspension comprises PBS, and optionally, sucrose or glucose).

65. The pharmaceutical composition of claim 63, wherein the suspension is for intravenous administration (e.g., the suspension comprises PBS).

66. The pharmaceutical composition of claim 63, wherein the suspension is for intradermal administration (e.g., the suspension comprises PBS).

67. The pharmaceutical composition of any one of claims 63-66, wherein the suspension further comprises a pharmaceutically acceptable excipient.

68. The pharmaceutical composition of any one of claims 63-67, wherein the suspension further comprises a buffer (e.g., PBS).

69. The pharmaceutical composition of any one of claims 1-68, wherein the composition further comprises one or more additional therapeutic agents.

70. The pharmaceutical composition of claim 69, wherein the one or more additional therapeutic agents is selected from the group consisting of an immunosuppressive agent, a DMARD, a pain-control drug, a steroid, a non-steroidal anti-inflammatory drug (NSAID), a cytokine antagonist, cyclosporin, retinoids, corticosteroids, propionic acid derivative, acetic acid derivative, enolic acid derivatives, fenamic acid derivatives, Cox-2 inhibitors, lumiracoxib, ibuprofen, cholin magnesium salicylate, fenoprofen, salsalate, difunisal, tolmetin, ketoprofen, flurbiprofen, oxaprozin, indomethacin, sulindac, etodolac, ketorolac, nabumetone, naproxen, valdecoxib, etoricoxib, MK0966; rofecoxib, acetaminophen, Celecoxib, Diclofenac, tramadol, piroxicam, meloxicam, tenoxicam, droxicam, lornoxicam, isoxicam, mefanamic acid, meclofenamic acid, flufenamic acid, tolfenamic, valdecoxib, parecoxib, etodolac, indomethacin, aspirin, ibuprophen, firocoxib, methotrexate (MTX), antimalarial drugs, hydroxychloroquine, chloroquine, sulfasalazine, Leflunomide, azathioprine, cyclosporin, gold salts, minocycline, cyclophosphamide, D-penicillamine, minocycline, auranofin, tacrolimus, myocrisin, chlorambucil, TNF alpha antagonists, TNF alpha antagonists, TNF alpha receptor antagonists, ADALIMUMAB (Humira®), ETANERCEPT (Enbrel®), INFLIXIMAB (Remicade®; TA-650), CERTOLIZUMAB PEGOL (Cimzia®; CDP870), GOLIMUMAB (Simpom®; CNTO 148), ANAKINRA (Kineret®), RITUXIMAB (Rituxan®; MabThera®), ABATACEPT (Orencia®), TOCILIZUMAB (RoActemra /Actemra®), integrin antagonists, TYSABRI® (natalizumab), IL-1 antagonists, ACZ885 (Ilaris), Anakinra (Kineret®), CD4 antagonists, IL-23 antagonists, IL-20 antagonists, IL-6 antagonists, BLyS antagonists, Atacicept, Benlysta®/ LymphoStat-B® (belimumab), p38 Inhibitors, CD20 antagonists, Ocrelizumab, Ofatumumab (Arzerra®), interferon gamma antagonists, Fontolizumab, prednisolone, Prednisone, dexamethasone, Cortisol, cortisone, hydrocortisone, methylprednisolone, betamethasone, triamcinolone, beclometasome, fludrocortisone, deoxycorticosterone, aldosterone, Doxycycline, vancomycin, pioglitazone, SBI-087, SCIO-469, Cura-100, Oncoxin + Viusid, TwHF, Methoxsalen, Vitamin D - ergocalciferol, Milnacipran, Paclitaxel, rosig tazone, Tacrolimus, Prograf®, RADOOl, rapamune, rapamycin, fostamatinib, Fentanyl, XOMA 052, Fostamatinib disodium, rosightazone, Curcumin, Longvida™, Rosuvastatin, Maraviroc, ramipnl, Milnacipran, Cobiprostone, somatropin, tgAAC94 gene therapy vector, MK0359, GW856553, esomeprazole, everolimus, trastuzumab, JAKl and JAK2 inhibitors, pan JAK inhibitors, e.g., tetracyclic pyridone 6 (P6), 325, PF-956980, denosumab, IL-6 antagonists, CD20 antagonistis, CTLA4 antagonists, IL-8 antagonists, IL-21 antagonists, IL-22 antagonist, integrin antagonists, Tysarbri® (natalizumab), VGEF antagnosits, CXCL antagonists, MMP antagonists, defensin antagonists, IL-1 antagonists, IL-1 beta antagonsits, IL-23 antagonists, receptor decoys, antagonistic antibodies, corticosteroids, mesalazine, mesalamine, sulfasalazine, sulfasalazine derivatives, immunosuppressive drugs, cyclosporin A, mercaptopurine, azathiopurine, prednisone, methotrexate, antihistamines, glucocorticoids, epinephrine, theophylline, cromolyn sodium, anti-leukotrienes, anti-cholinergic drugs for rhinitis, TLR antagonists, inflammasome inhibitors, anti-cholinergic decongestants, mast-cell stabilizers, monoclonal anti-IgE antibodies, vaccines, cytokine inhibitors, anti-IL-6 antibodies, TNF inhibitors, palmitoylethanolamide, an inhibitor of N-Acylethanolamine Acid Amidase (NAAA), interferon-β, glatiramer acetate, mitoxantrone, and glucocorticoids.

71. The pharmaceutical composition of claim 69, wherein the one or more additional therapeutic agents is selected from the group consisting of an immunosuppressive agent, a non-steroidal anti-inflammatory drug (NSAID), palmitoylethanolamide, an inhibitor of N-Acylethanolamine Acid Amidase (NAAA), interferon-β, glatiramer acetate, mitoxantrone, and glucocorticoids.

72. The pharmaceutical composition of claim 69, wherein the one or more additional therapeutic agents is an antibiotic, optionally wherein the antibiotic is selected from the group consisting of aminoglycosides, ansamycins, carbacephems, carbapenems, cephalosporins, glycopeptides, lincosamides, lipopeptides, macrolides, monobactams, nitrofurans, oxazolidonones, penicillins, polypeptide antibiotics, quinolones, fluoroquinolone, sulfonamides, tetracyclines, anti-mycobacterial compounds and combinations thereof.

73. The pharmaceutical composition of any one of claims 1-72, wherein the pharmaceutical composition is formulated for a daily dose.

74. The pharmaceutical composition of any one of claims 1-72, wherein the pharmaceutical composition is formulated for twice a day dose, wherein each dose is half of the daily dose.

75. The pharmaceutical composition of any one of claims 1-74 for use in treating a disease (e.g., a neuroinflammatory disease, a neurodegenerative disease, a neuromuscular disease, and/or a psychiatric disorder).

76. Use of a pharmaceutical composition of any one of claims 1-74 for the preparation of a medicament for the treatment of a disease (e.g., a neuroinflammatory disease, a neurodegenerative disease, a neuromuscular disease, and/or a psychiatric disorder).

77. A method of treating a subject (e.g., human) in need thereof (e.g., afflicted with a disease, e.g., a neuroinflammatory disease, a neurodegenerative disease, a neuromuscular disease, and/or a psychiatric disorder), comprising administering to the subject a pharmaceutical composition of any one of claims 1-74.

78. The method of claim 77, wherein the subject is in need of treatment for a neuroinflammatory disease.

79. The method of claim 77, wherein the subject is in need of treatment for a neurodegenerative disease and/or a neuromuscular disease.

80. The method of claim 77, wherein the subject is in need of treatment for a psychiatric disorder.

81. The method of any one of claims 77-80, wherein the subject is in need of treatment for a disease selected from, encephalitis, encephalomyelitis, meningitis, Guillain-Barre syndrome, neuromyotonia, narcolepsy, multiple sclerosis, myelitis, schizophrenia, acute disseminated encephalomyelitis (ADEM), accute optic neuritis (AON), transverse myelitis, neuromyelitis optica (NMO), Alzheimer’s disease, Parkinson’s disease, amyotrophic lateral sclerosis, frontotemporal lobar dementia, optic neuritis, neuromyelitis optica spectrum disorder (NMOSD), autoimmune encephalitis, anti-NMDA receptor encephalitis, Rasmussen’s encephalitis, acute necrotizing encephalopathy of childhood (ANEC), opsoclonus-myoclonus ataxia syndrome, traumatic brain injury, Huntington’s disease, depression, anxiety, migraine, myasthenia gravis, acute ischemic stroke, epilepsy, synucleinopathies, frontotemporal dementia, progressive nonfluent aphasia, semantic dementia, Nodding syndrome, cerebral ischemia, neuropathic pain, autism spectrum disorder, fibromyalgia syndrome, progressive supranuclear palsy, corticobasal degeneration, systemic lupus erythematosus, prion disease, motor neuron diseases (MND), spinocerebellar ataxia, spinal muscular atrophy, dystonia, idiopathicintracranial hypertension, nervous system disease, central nervous system disease, peripheral nervous system disease, movement disorders, encephalopathy, peripheral neuropathy, and post-operative cognitive dysfunction.

82. The method of any one of claims 77-81, wherein the subject is in need of treatment for a disease selected from encephalitis, encephalomyelitis, meningitis, multiple sclerosis, schizophrenia, acute disseminated encephalomyelitis (ADEM), accute optic neuritis (AON), transverse myelitis, neuromyelitis optica (NMO), Alzheimer’s disease, Parkinson’s disease, amyotrophic lateral sclerosis, frontotemporal lobar dementia, traumatic brain injury, Huntington’s disease, depression, anxiety, migraine, acute ischemic stroke, epilepsy, synucleinopathies, semantic dementia, cerebral ischemia, neuropathic pain, autism spectrum disorder, peripheral neuropathy, motor neuron diseases (MND), spinocerebellar ataxia, spinal muscular atrophy, and fibromyalgia syndrome.

83. The method of any one of claims 77-82, further comprising administering to the subject one or more additional therapeutic agents.

84. The method of claim 83, wherein the one or more additional therapeutic agents is selected from the group consisting of an immunosuppressive agent, a DMARD, a pain-control drug, a steroid, a non-steroidal anti-inflammatory drug (NSAID), a cytokine antagonist, cyclosporin, retinoids, corticosteroids, propionic acid derivative, acetic acid derivative, enolic acid derivatives, fenamic acid derivatives, Cox-2 inhibitors, lumiracoxib, ibuprofen, cholin magnesium salicylate, fenoprofen, salsalate, difunisal, tolmetin, ketoprofen, flurbiprofen, oxaprozin, indomethacin, sulindac, etodolac, ketorolac, nabumetone, naproxen, valdecoxib, etoricoxib, MK0966; rofecoxib, acetaminophen, Celecoxib, Diclofenac, tramadol, piroxicam, meloxicam, tenoxicam, droxicam, lornoxicam, isoxicam, mefanamic acid, meclofenamic acid, flufenamic acid, tolfenamic, valdecoxib, parecoxib, etodolac, indomethacin, aspirin, ibuprophen, firocoxib, methotrexate (MTX), antimalarial drugs, hydroxychloroquine, chloroquine, sulfasalazine, Leflunomide, azathioprine, cyclosporin, gold salts, minocycline, cyclophosphamide, D-penicillamine, minocycline, auranofin, tacrolimus, myocrisin, chlorambucil, TNF alpha antagonists, TNF alpha antagonists, TNF alpha receptor antagonists, ADALIMUMAB (Humira®), ETANERCEPT (Enbrel®), INFLIXIMAB (Remicade®; TA-650), CERTOLIZUMAB PEGOL (Cimzia®; CDP870), GOLIMUMAB (Simpom®; CNTO 148), ANAKINRA (Kineret®), RITUXIMAB (Rituxan®; MabThera®), ABATACEPT (Orencia®), TOCILIZUMAB (RoActemra /Actemra®), integrin antagonists, TYSABRI® (natalizumab), IL-1 antagonists, ACZ885 (Ilaris), Anakinra (Kineret®), CD4 antagonists, IL-23 antagonists, IL-20 antagonists, IL-6 antagonists, BLyS antagonists, Atacicept, Benlysta®/ LymphoStat-B® (belimumab), p38 Inhibitors, CD20 antagonists, Ocrelizumab, Ofatumumab (Arzerra®), interferon gamma antagonists, Fontolizumab, prednisolone, Prednisone, dexamethasone, Cortisol, cortisone, hydrocortisone, methylprednisolone, betamethasone, triamcinolone, beclometasome, fludrocortisone, deoxycorticosterone, aldosterone, Doxycycline, vancomycin, pioglitazone, SBI-087, SCIO-469, Cura-100, Oncoxin + Viusid, TwHF, Methoxsalen, Vitamin D - ergocalciferol, Milnacipran, Paclitaxel, rosig tazone, Tacrolimus, Prograf®, RADOOl, rapamune, rapamycin, fostamatinib, Fentanyl, XOMA 052, Fostamatinib disodium, rosightazone, Curcumin, Longvida™, Rosuvastatin, Maraviroc, ramipnl, Milnacipran, Cobiprostone, somatropin, tgAAC94 gene therapy vector, MK0359, GW856553, esomeprazole, everolimus, trastuzumab, JAKl and JAK2 inhibitors, pan JAK inhibitors, e.g., tetracyclic pyridone 6 (P6), 325, PF-956980, denosumab, IL-6 antagonists, CD20 antagonistis, CTLA4 antagonists, IL-8 antagonists, IL-21 antagonists, IL-22 antagonist, integrin antagonists, Tysarbri® (natalizumab), VGEF antagnosits, CXCL antagonists, MMP antagonists, defensin antagonists, IL-1 antagonists, IL-1 beta antagonsits, IL-23 antagonists, receptor decoys, antagonistic antibodies, corticosteroids, mesalazine, mesalamine, sulfasalazine, sulfasalazine derivatives, immunosuppressive drugs, cyclosporin A, mercaptopurine, azathiopurine, prednisone, methotrexate, antihistamines, glucocorticoids, epinephrine, theophylline, cromolyn sodium, anti-leukotrienes, anti-cholinergic drugs for rhinitis, TLR antagonists, inflammasome inhibitors, anti-cholinergic decongestants, mast-cell stabilizers, monoclonal anti-IgE antibodies, vaccines, cytokine inhibitors, anti-IL-6 antibodies, TNF inhibitors, palmitoylethanolamide, an inhibitor of N-Acylethanolamine Acid Amidase (NAAA), interferon-β, glatiramer acetate, mitoxantrone, and glucocorticoids.

85. The method of claim 83, wherein the one or more additional therapeutic agents is selected from the group consisting of an immunosuppressive agent, a non-steroidal anti-inflammatory drug (NSAID), palmitoylethanolamide, an inhibitor of N-Acylethanolamine Acid Amidase (NAAA), interferon-β, glatiramer acetate, mitoxantrone, and glucocorticoids.

86. The method of claim 83, wherein the one or more additional therapeutic agents is an antibiotic, optionally wherein the antibiotic is selected from the group consisting of aminoglycosides, ansamycins, carbacephems, carbapenems, cephalosporins, glycopeptides, lincosamides, lipopeptides, macrolides, monobactams, nitrofurans, oxazolidonones, penicillins, polypeptide antibiotics, quinolones, fluoroquinolone, sulfonamides, tetracyclines, anti-mycobacterial compounds and combinations thereof.

87. The method of any one of claims 77-86, wherein the pharmaceutical composition is administered orally, rectally, sublingually, intradermally, intravenously, intraperitoneally, or subcutaneously.

88. The method of any one of claims 77-87, wherein the pharmaceutical composition is administered by injection, e.g., subcutaneous, intradermal, or intraperitoneal injection.

89. The method of any one of claims 77-88, wherein the pharmaceutical composition is administered intravenously.

90. The method of any one of claims 77-88, wherein the pharmaceutical composition is administered intradermally.

91. The method of any one of claims 77-87, wherein the pharmaceutical composition is administered orally.

92. The method of any one of claims 77-91, wherein the pharmaceutical composition further comprises one or more additional therapeutic agents.

93. The method of any one of claims 77-92, wherein the dose of mEVs in the pharmaceutical composition is about 2×10⁶ to about 2×10¹⁶ particles (e.g., wherein particle count is determined by NTA (nanoparticle tracking analysis)).

94. The method of any one of claims 77-93, wherein the dose of mEVs in the pharmaceutical composition is 5 mg to about 900 mg total protein (e.g., wherein total protein is determined by Bradford assay or BCA).

95. The method of any one of claims 77-94, wherein the pharmaceutical composition is administered once a day.

96. The method of any one of claims 77-94, wherein the pharmaceutical composition is administered twice a day.

97. The method of any one of claims 77-94, wherein the pharmaceutical composition is formulated for a daily dose.

98. The method of any one of claims 77-94, wherein the pharmaceutical composition is formulated for twice a day dose, wherein each dose is half of the daily dose.
